(19) United States
(12) Patent Application Publication
(10) Pub. No.: US 2022/0380454 A1
(43) Pub. Date: Dec. 1, 2022

(54) METHODS AND COMPOSITIONS FOR TREATING ALZHEIMER'S DISEASE

(71) Applicant: The Regents of the University of California, Oakland, CA (US)

(72) Inventors: Yimin Zou, La Jolla, CA (US); Bo Feng, La Jolla, CA (US); Runyi Tian, La Jolla, CA (US); Andiara Freitas, La Jolla, CA (US)

(21) Appl. No.: 17/623,192

(22) PCT Filed: Jun. 23, 2020
(86) PCT No.: PCT/US2020/039194

§ 371 Date: Dec. 27, 2021

Related U.S. Application Data
(60) Provisional application No. 62/868,407, filed on Jun. 28, 2019.
(60) Provisional application No. 63/019,970, filed on May 4, 2020.

(51) Int. Cl. C07K 16/28; A61K 38/17; C07K 16/18; G01N 33/50

(52) U.S. Cl. CPC C07K 16/28; A61K 38/177; C07K 16/18; G01N 33/5058; G01N 33/5041; C07K 2319/30; C07K 2317/76

(57) ABSTRACT

Provided herein are methods and agents for modulating the signaling pathway and components thereof that are responsible for assembly and disassembly of synapses in neurons, including amyloid beta (Aβ) mediated synaptotoxicity and synapse loss. Also provided herein are methods for screening and identifying candidate agents capable of modulating synapse formation and (Aβ) mediated synaptotoxicity.

### CROSS REFERENCE TO RELATED APPLICATION(S)

This application claims the benefit of priority under 35 U.S.C. § 119(e) of U.S. Ser. No. 62/868,407, filed Jun. 28, 2019, and of U.S. Ser. No. 63/019,970, filed May 4, 2020, the entire content of each of which is incorporated herein by reference.

### GRANT INFORMATION

The present invention was made with government support under Grant No. MH116667 awarded by the National Institutes of Health. The United States government has certain rights in the invention.

### SEQUENCE LISTING

The instant application contains a Sequence Listing which has been submitted electronically in ASCII format and is hereby incorporated by reference in its entirety. Said ASCII copy, created on Jun. 22, 2020, is named 20378-202583_SL.txt and is 115 kilobytes in size.

### BACKGROUND OF THE INVENTION

### Field of the Invention

The present disclosure generally relates to the signaling pathways that modulate synaptic formation and maintenance in neurons, and the molecular mechanism underlying amyloid beta (Aβ) oligomer-mediated synaptotoxicity and related diseases and conditions. The present disclosure also relates to methods and agents for modulating the assembly and disassembly of synapses in neurons, and for the management, prevention and treatment of neurodegenerative diseases.

### Background Information

Loss of glutamatergic synapses is an important early step in pathogenesis of Alzheimer's disease and is thought to be induced by oligomeric amyloid β (Aβ). How β amyloid leads to glutamatergic synapse loss is previously unknown. The approaches to reduce β amyloid production or clearing are prone to side effects as the enzymes and the cellular processes that produce β amyloid have important functions in many tissues and β amyloid itself has normal physiological functions. There are unmet needs for the better understanding of molecular mechanisms underlying Aβ-mediated synaptotoxicity and ensuing conditions and disease, as well as methods for the identification and designing of effective modulators for research and therapeutic uses based on the mechanisms. There are also unmet needs for the provisions of therapeutic methods and agents for preventing, managing and treating conditions and diseases associated with Aβ-mediated synaptotoxicity, such as neurodegenerative diseases resulted from loss of synapses. The present disclosure meets these needs.

### SUMMARY OF THE INVENTION

Provided herein are methods and agents for modulating the signaling pathway and components thereof that are responsible for assembly and disassembly of synapses in neurons, including amyloid beta (Aβ) mediated synaptotoxicity and synapse loss. Also provided herein are methods for screening and identifying candidate agents capable of modulating synapse formation and (Aβ) mediated synaptotoxicity.

In one aspect, provided herein are methods for reducing or preventing amyloid beta (Aβ) induced loss of synapses in a population of neurons. In some embodiments, the method for reducing or preventing amyloid beta (Aβ) induced loss of synapses in a population of neurons includes contacting the neurons with an effective amount of an Aβ inhibitor that blocks binding of Aβ to Celsr.

In some embodiments, the Aβ inhibitor competes with Aβ for binding to the EGF7, EGF8, and/or Laminin G1 domains of Celsr. In some embodiments, the Aβ inhibitor competes with Aβ for binding to the Laminin G1 domain of Celsr.

In some embodiments, the Aβ is oligomeric Aβ. In some embodiments, the Aβ inhibitor is an anti-Celsr antibody or antigen binding fragment thereof. In some embodiments, the antibody or antigen binding fragment binds to an epitope in the EGF7, EGF8, or Laminin G1 domain of Celsr. In some embodiments, the anti-Celsr antibody specifically binds to Celsr. In some embodiments, the anti-Celsr antibody preferentially binds to Celsr3 over Celsr2.

In some embodiments, the Aβ inhibitor competes with Celsr for binding with Aβ. In-some embodiments, the Aβ inhibitor is an antibody or antigen binding fragment thereof that binds to Aβ. In some embodiments, the Aβ inhibitor comprises a Celsr3 peptide. In some embodiments, the Celsr3 peptide comprises (a) one or more Laminin G1 domain of Celsr or a functional variant thereof, (b) one or more EGF7 domain of Celsr or a functional variant thereof, (c) one or more EGF8 domain of Celsr or a functional variant thereof, (d) one or more extracellular domain of Celsr or a function variant thereof, or (e) any combination of (a) to (d). In some embodiments, the Aβ inhibitor comprises a Celsr3 peptide fused to an immunoglobulin Fc region.

In some embodiments, the Celsr peptide comprises the Laminin G1 domain of Celsr3 having the amino acid sequence set forth in SEQ ID NO: 35 or SEQ ID NO: 36, or a functional variant thereof. In some embodiments, the functional variant has an amino acid sequence having at least about 50%, 60%, 70%, 80%, 90%, 95% or 98% sequence homology to SEQ ID NO: 35 or SEQ ID NO: 36.

In some embodiments, the Celsr peptide comprises the EGF7 domain of Celsr3 having the amino acid sequence set forth in SEQ ID NO: 37 or SEQ ID NO: 38, or a functional variant thereof. In some embodiments, the functional variant has an amino acid sequence having at least about 50%, 60%, 70%, 80%, 90%, 95% or 98% sequence homology to SEQ ID NO: 37 or SEQ ID NO: 38.

In some embodiments, the Celsr peptide comprises the EGF8 domain of Celsr3 having the amino acid sequence set forth in SEQ ID NO: 39, or a functional variant thereof. In some embodiments, the functional variant has an amino acid sequence having at least about 50%, 60%, 70%, 80%, 90%, 95% or 98% sequence homology to SEQ ID NO: 39.

In some embodiments, the method further comprises contacting the population of neurons with a Ryk inhibitor. In some embodiments, the method further comprises contacting the population of neurons with a Vangl inhibitor. In some embodiments, the method further comprises contacting the population of neurons with a Celsr agonist. In some embodiments, the method further comprises contacting the population of neurons with a Frizzled agonist.

In another aspect, provided herein are methods of modulating formation of synapses in a population of neurons. In some embodiments, the methods of modulating formation of synapses in a population of neurons include modulating one or more planar cell polarity (PCP) signaling pathway component and/or one or more components of a Wnt-mediated signaling pathway. In particular embodiments, the Wnt-mediated signaling pathway is the non-canonical Wnt signaling pathway.

In some embodiments, the PCP signaling pathway component is selected from the group consisting of Celsr, Frizzled and Vangl. In some embodiments, the non-canonical Wnt signaling pathway component is Ryk.

In some embodiments, the step of modulating comprises contacting the neurons with a Ryk inhibitor. In some embodiments, the step of modulating comprises contacting the neurons with a Vangl inhibitor. In some embodiments, the step of modulating comprises contacting the neurons with a Celsr agonist. In some embodiments, the step of modulating comprises contacting the neurons with a Frizzled agonist.

In some embodiments, the Ryk inhibitor reduces or inhibits Ryk binding to Wnt. In some embodiments, the Ryk inhibitor is an anti-Ryk antibody or antigen binding fragment thereof. In some embodiments, the Ryk inhibitor inhibits or reduces Ryk expression in the neurons.

In some embodiments, the Vangl inhibitor reduces or inhibits expression of Vangl in the neurons. In some embodiments, the Vangl inhibitor reduces or inhibits binding of Vangl to (a) Celsr; (b) Frizzled; and/or (c) a complex comprising Celsr and Frizzled. In some embodiments, the Vangl inhibitor reduces or inhibits Vangl from disrupting intracellular complexes formed by Celsr at the presynaptic and postsynaptic membranes of a synapse.

In some embodiments, the Celsr agonist (a) increases Celsr expression; (b) reduces endocytosis of Celsr located in synaptic sites of the neurons; and/or (c) increases transportation of Celsr to synaptic sites in the neurons.

In some embodiments, the Frizzled agonist (a) increases Frizzled expression; (b) reduces endocytosis of Frizzled located in synaptic sites of the neurons; and/or (c) increases transportation of Frizzled to synaptic sites in the neurons.

In some embodiments, the population of neurons are in a subject, and any of the contacting step is performed by administering the (a) Aβ inhibitor, (b) Ryk inhibitor, (c) Vangl inhibitor, (d) Celsr agonist, and/or (e) Frizzled agonist to the subject. In some embodiments, the population of neurons is in the brain of the subject. In some embodiments, the subject has or is at risk of developing a neurodegenerative disease.

In a related aspect, provided herein are also methods for managing, preventing, or treating a neurodegenerative disease in a subject. In some embodiments, the method for managing, preventing, or treating a neurodegenerative disease in a subject includes administering to the subject a therapeutically effective amount of an amyloid beta (Aβ) inhibitor that blocks binding of Aβ to Celsr. In some embodiments, the Aβ inhibitor competes with Aβ for binding to EGF7, EGF8, and/or Laminin G1 domains of Celsr.

In some embodiments, the Aβ is oligomeric Aβ. In some embodiments, the Aβ inhibitor is an anti-Celsr antibody or antigen binding fragment thereof. In some embodiments, the antibody or antigen binding fragment binds to an epitope in the EGF7, EGF8, or Laminin G1 domain of Celsr.

In some embodiments, the anti-Celsr antibody specifically binds to Celsr3. In some embodiments, the anti-Celsr antibody preferentially binds to Celsr3 over Celsr2.

In some embodiments, the Aβ inhibitor competes with Celsr3 for binding with Aβ. In some embodiments, the Aβ inhibitor is an antibody or antigen binding fragment thereof that binds to Aβ. In some embodiments, the Aβ inhibitor comprises a Celsr3 peptide. In some embodiments, the Celsr3 peptide comprises (a) one or more Laminin G1 domain of Celsr or a functional variant thereof, (b) one or more EGF7 domain of Celsr or a functional variant thereof, (c) one or more EGF8 domain of Celsr or a functional variant thereof, (d) one or more extracellular domain of Celsr or a function variant thereof, or (e) any combination of (a) to (d). In some embodiments, the Aβ inhibitor comprises a Celsr3 peptide fused to an immunoglobulin Fc region.

In some embodiments, the Celsr3 peptide comprises the Laminin G1 domain of Celsr having the amino acid sequence set forth in SEQ ID NO: 35 or SEQ ID NO: 36, or a functional variant thereof. In some embodiments, the functional variant has an amino acid sequence having at least about 50%, 60%, 70%, 80%, 90%, 95% or 98% sequence homology to SEQ ID NO: 35 or SEQ ID NO: 36.

In some embodiments, the Celsr3 peptide comprises the EGF7 domain of Celsr having the amino acid sequence set forth in SEQ ID NO: 37 or SEQ ID NO: 38, or a functional variant thereof. In some embodiments, the functional variant has an amino acid sequence having at least about 50%, 60%, 70%, 80%, 90%, 95% or 98% sequence homology to SEQ ID NO: 37 or SEQ ID NO: 38.

In some embodiments, the Celsr3 peptide comprises the EGF8 domain of Celsr having the amino acid sequence set forth in SEQ ID NO: 39, or a functional variant thereof. In some embodiments, the functional variant has an amino acid sequence having at least about 50%, 60%, 70%, 80%, 90%, 95% or 98% sequence homology to SEQ ID NO: 39.

In some embodiments, the method further comprises administering to the subject at least one additional therapeutic agent selected from a Ryk inhibitor, a Vangl inhibitor, a Celsr agonist, or a Frizzled agonist.

In some embodiments, the Ryk inhibitor reduces or inhibits Ryk binding to Wnt. In some embodiments, the Ryk inhibitor is an anti-Ryk antibody or antigen binding fragment thereof. In some embodiments, the Ryk inhibitor inhibits or reduces Ryk expression in the neurons.

In some embodiments, the Vangl inhibitor reduces or inhibits expression of Vangl in the neurons. In some embodiments, the Vangl inhibitor reduces or inhibits binding of Vangl to (a) Celsr, (b) Frizzled, and/or (c) a complex comprising Celsr and Frizzled. In some embodiments, the Vangl inhibitor reduces or inhibits Vangl from disrupting intracellular complexes formed by Celsr3 at the presynaptic and postsynaptic membranes of a synapse.

In some embodiments, the Celsr agonist (a) increases Celsr expression; (b) reduces endocytosis of membrane-associated Celsr; and/or (c) increases transportation of Celsr to synaptic sites in the neurons.

In some embodiments, the Frizzled agonist (a) increases Frizzled expression; (b) reduces endocytosis of membrane-associated Frizzled; and/or (c) increases transportation of Frizzled to synaptic sites in the neurons.

In some embodiments, the neurodegenerative disease is Alzheimer's disease or Parkinson's disease. In some embodiments, the number of synapses in a population of neurons in the subject is increased.

In some embodiments, the present methods for reducing or preventing amyloid beta (Aβ) induced loss of synapses in a population of neurons, or the present methods for reducing or preventing amyloid beta (Aβ) induced loss of synapses in a population of neurons, or the present methods for managing, preventing, or treating a neurodegenerative disease in a subject are capable of resulting in one or more effects in the population of neurons treated with the modulators and therapeutic agents described herein.

In some embodiments, the present method is capable of increasing the amount or number of complexes comprising Celsr and Frizzled in the population of neurons is increased. In some embodiments, the complex further comprises Vangl. In some embodiments, the complex further comprises Ryk.

In some embodiments, formation of the complex is mediated by the Laminin G1 domains of Celsr. In some embodiments, disassembly of the complex is mediated by Vangl.

In some embodiments, the complex comprises Celsr and Frizzled co-expressed in a first neuron of the population of neurons. In some embodiments, the complex further comprises Celsr expressed in a second neuron in the population of neurons. In some embodiments, the complex is formed via interaction between the extracellular domains of Celsr expressed by the first and second neurons. In some embodiments, the extracellular domain is the Laminin G1 domain of Celsr. In some embodiments, the first and second neurons form synapses and the complex is located at the synapses.

In some embodiments, the complex comprises presynaptic Celsr and presynaptic Frizzled. In some embodiments, the complex further comprises postsynaptic Celsr. In some embodiments, the complex further comprises presynaptic Ryk. In some embodiments, the complex further comprises postsynaptic Vangl. In some embodiments, the complex stabilizes synapses in the population of neurons.

In some embodiments, the present method is capable of increasing the amount of Frizzled located at the presynaptic site. In some embodiments, the present method is capable of increasing the amount of Celsr located at presynaptic site and/or at postsynaptic site.

In some embodiments, the present method is capable of increasing the number of synapses in the population of neurons. In some embodiments, the synapses are excitatory synapses. In some embodiments, the synapses are glutamatergic synapses. In some embodiments, the population of neurons comprises a cerebellar granule neuron, a dorsal root ganglion neuron, a cortical neuron, a sympathetic neuron, or a hippocampal neuron.

In another aspect, provided herein are also methods for selecting an agent capable of modulating of synapse formation in a population of neurons. In some embodiments, the method includes providing a population of cells comprising a first cell expressing Frizzled and Celsr and a second cell expressing Vangl; measuring a first level of association between Celsr and Frizzled; contacting a candidate agent with the population of cells; measuring a second level of association between Celsr and Frizzled; and selecting the candidate agent as the modulator if the second level of association is different from the first level of association.

In some embodiments, the Celsr or Celsr variant is expressed on the surface of a cell. In some embodiments, the second cell further expresses Celsr. In some embodiments, the population of cells are neurons. In some embodiments, the second cell further expresses Celsr. In some embodiments, the first cell further expresses Ryk.

In some embodiments, the step of measuring comprises measuring the binding affinity between Celsr and Frizzled. In some embodiments, the step of measuring comprises measuring the binding affinity between Celsr and Vangl.

In some embodiments, the step of measuring is performed by measuring the amount of complexes comprising Celsr and Frizzled in the population of cells. In some embodiments, the amount of complexes is measured by co-immunoprecipitation of Celsr and Frizzled from the population of cells. In some embodiments, the amount of complexes is measured by co-immunoprecipitation of Celsr and Vangl from the population of cells.

In some embodiments, the step of measuring is performed by measuring the level of colocalization of Celsr and Frizzled in the cells. In some embodiments, the population of cells are neurons forming synapses, and the colocalization of Celsr and Frizzled is at synaptic sites of the neurons. In some embodiments, the step of measuring the level of colocalization is performed by visualizing Celsr and Frizzled via microscopy.

In some embodiments, the population of cells are neurons and the step of measuring comprises measuring the amount of Celsr located at synaptic sites in the neurons. In some embodiments, the population of cells are neurons and step of the measuring comprises measuring the amount of Frizzled located at synaptic sites in the neurons. In some embodiments, the measuring comprises visualizing Celsr or Frizzled via microscopy. In some embodiments, the measuring further comprises visualizing a synaptic marker via microscopy. In some embodiments, the population of cells are neurons and step of the measuring is performed by measuring the number of synapses formed in the neurons.

In some embodiments, the candidate agent comprises a small-molecule compound, a nucleic acid, or a peptide. In some embodiments, the candidate agent comprises a microRNA, siRNA or CRISPR-based gene editing construct. In some embodiments, the candidate agent is an antibody or antigen binding fragment thereof.

In some embodiments, the method is performed in the presence of oligomeric Aβ. In some embodiments, the method is performed in the presence of Wnt.

In some embodiments, the genome of the cells comprises a heterozygous or homozygous deletion, inactivation or knock-out of the Ryk gene. In some embodiments, the genome of the cells further comprises a heterozygous or homozygous deletion, inactivation or knock-out of the Vangl gene.

In some embodiments, the population of the cells is in a non-human mammal, and the step of contacting is performed by administering the candidate agent to the non-human mammal.

In yet another aspect, provided herein are also methods for selecting an amyloid beta (Aβ) inhibitor that prevents or reduces Aβ-mediated neurotoxicity. In specific embodiments, the method of selecting an amyloid beta (Aβ) inhibitor that prevents or reduces Aβ-mediated neurotoxicity comprises contacting a candidate agent with Celsr or a Celsr variant in the presence of Aβ; and selecting the candidate agent as the Aβ inhibitor if the candidate agent reduces or inhibits binding of Aβ to the Celsr or Celsr variant.

In some embodiments, the Celsr or Celsr variant is expressed on the surface of a cell. In some embodiments, the cell is a neuron. In some embodiments, the cell is in an in vitro cell culture. In some embodiments, the cell is in a non-human mammal cell. In some embodiments, the Celsr or Celsr variant is immobilized on a solid support.

In some embodiments, the Celsr variant comprises a deletion of (a) one or more Celsr cadherin domains; (b) one or more Celsr EFG domains selected from EFG1, EFG2, EFG3, EFG4, EFG5, and EFG6; (c) one or more of Celsr laminin domains selected from Laminin-G2 and Laminin-G3; or (d) any combination of (a) to (c).

In some embodiments, the Celsr variant consists essentially of one or more extracellular domains of Celsr selected from EFG7, EFG8, and Laminin-G1. In some embodiments, the Aβ is oligomeric Aβ comprising about 2-5 Aβ monomers.

In some embodiments, the candidate agent comprises a small-molecule compound, a nucleic acid, or a peptide. In some embodiments, the candidate agent is an anti-Celsr antibody or antigen binding fragment thereof. In some embodiments, the antibody or antigen binding fragment binds to an epitope in the EFG7, EFG8, or Laminin-G1 domain of Celsr. In some embodiments, the candidate agent is an anti-Aβ antibody or antigen binding fragment thereof. In some embodiments, the candidate agent is a member of a candidate agent library.

In some embodiments, the method further comprises administering the selected candidate agent into a subject having or at risk of developing a neurodegenerative disease. In some embodiments, the number neuronal synapses in the subject is increased. In some embodiments, the neurodegenerative disease is prevented or treated. In some embodiments, the neurodegenerative disease is Alzheimer's disease or Parkinson's disease. In some embodiments, the Celsr is Celsr3. In some embodiments, the Frizzled is Frizzled3. In some embodiments, the Vangl is Vangl2.

### BRIEF DESCRIPTION OF THE DRAWINGS

FIG. 1 shows characterization of Aβ oligomers. Total Aβ42 oligomers were separated from Aβ42 monomer in 12% SDS-PAGE Gel. Aβ42 oligomers were composed by different sizes of oligomers ranging from 2-mer to 4-mer.

FIG. 2 demonstrates that Vangl2 is involved in Aβ oligomer-induced synapse loss in vitro and in vivo. Particularly, FIG. 2A is a schematic illustrating the experimental design for generating the data shown in FIGS. 2B through 2D. Hippocampal neurons were challenged by AAV-Cre virus on DIV7 for 7 days and then were challenged by oligomeric Aβ42; FIG. 2B shows AAV-Cre virus induced knock-down of Vangl2 expression in Vangl2^{fl/fl} hippocampal neuron; FIG. 2C and FIG. 2D show immunostaining for pre-(green) and postsynaptic (red) puncta of glutamatergic synapses (arrowheads) in 14-DIV hippocampal cultures from littermate Vangl2^{+/+} and Vangl2^{fl/fl} with or without oligomeric Aβ challenge. n=3 of Vangl2^{+/+} mice, n=4 of Vangl2^{fl/fl} from 3 independent experiments. *P<0.05, **P<0.01 and ***P<0.001, One-way ANOVA; FIG. 2E is a schematic illustrating the experimental design for the data shown in FIGS. 2F and 2G. AAV-Cre virus was injected into bilateral CA1 region for 2 weeks followed by oligomeric Aβ injection into bilateral ventricular for 5 days; FIG. 2F and FIG. 2G are representative images of Bassoon (red)- and PSD95 (green)-immunoreactive puncta (arrowheads) in stratum radiatum of Vangl2^{+/+} and Vangl2^{fl/fl} hippocampus (CA1) with or without oligomeric Aβ injection. * P<0.05, ** P<0.01, One-way ANOVA. n=8 of Vangl2^{+/+} mice, n=3 of Vangl2^{+/+} mice with oligomeric Aβ injection, n=6 of Vangl2^{fl/fl} mice and n=5 of Vangl2^{fl/fl} mice with oligomeric Aβ injection. * P<0.05, ** P<0.01, One-way ANOVA. Scale bar 5 μm in FIGS. 2C and 2F. Means±SEM.

FIG. 3 shows total expression of Celsr3 and Vangl2 in primary cultured neuron with oligomeric Aβ42 treatment.

FIG. 4A to FIG. 4I demonstrate Vangl2 disrupts the intercellular complex of Celsr3/Frizzled3-Celsr3. Particularly, FIG. 4A shows schematics illustrating the experimental design of intercellular interaction testing. FIG. 4B shows Co-IP assays testing the interaction between Celsr3 and Frizzled3 with Vangl2 in the neighboring cell. FIG. 4C shows quantification data of the expression level of co-IPed Celsr3. **P<0.01, Student's t test. FIG. 4D shows Co-IP assays testing the interaction between Celsr3 and Frizzled3 with Celsr3 in the neighboring cell. Student's t test. FIG. 4E shows quantification data of the expression level of co-IPed Celsr3. FIG. 4F shows schematics illustrating the experimental design of intercellular interaction testing. FIG. 4G shows Co-IP assays testing the intercellular complex between Celsr3/Frizzled3 in one cell and Celsr3 in the neighboring cell. FIG. 4H shows quantification data of the expression level of co-IPed Celsr3. ***P<0.001, Student's t test. FIG. 4I shows schematics of protein-protein interactions and intercellular complex. Means±SEM.

FIG. 4J to FIG. 4O demonstrate that Oligomeric Aβ disrupts the interaction between Frizzled3 and Celsr3 in the presence of Vangl2. FIG. 4J shows Schematics illustrating the experimental design. FIG. 4K shows IP assays testing the interaction between Celsr3 and Frizzled3 transfected in the same cell with or without oligomeric Aβ42. FIG. 4L shows Quantification data of the expression level of co-IPed Celsr3. Student's t test. FIG. 4M shows Schematics illustrating the experimental design. FIG. 4N shows IP assays testing the interaction between Celsr3 and Frizzled3, with or without oligomeric Aβ42 and/or Vangl2. FIG. 4O shows quantification data of the expression level of co-IPed Celsr3. *P<0.05 and ***P<0.001. One-way ANOVA. Means±SEM.

FIG. 4P to FIG. 4V demonstrate that Oligomeric Aβ causes synapse loss by tipping the balance of opposing functions of Celsr3 and Vangl2. FIG. 4P shows schematics illustrating the experimental design of intercellular interaction testing. FIG. 4Q shows IP assays testing the effects of oligomeric Aβ42 on intercellular complex between Celsr3/Frizzled3 in one cell and Celsr3 in the neighboring cell. FIG. 4R shows Quantification data of the expression level of co-IPed Celsr3. *P<0.05. Student's t test. FIG. 4S shows Schematics illustrating the experimental design of intercellular interaction testing. FIG. 4T shows IP assays showing that oligomeric Aβ42 enhances the function of Vangl2 in disrupting the intercellular complex between Celsr3/Frizzled3 in one cell and Celsr3 in the neighboring cell. FIG. 4U shows Quantification data of the expression level of co-IPed Frizzled3. *P<0.05 and ***P<0.001. One-way ANOVA. FIG. 4V shows schematics of protein-protein interactions and intercellular complex. Means±SEM.

FIG. 5 demonstrates that Celsr3 is a receptor for oligomeric Aβ. Particularly, FIG. 5A shows Vangl2-Flag (red), Frizzled3-HA (red), Celsr3-Flag (red)-transfected or control empty vector-transfected HEK293T cells were treated with oligomeric Aβ42 (200 nM total peptide, monomer equivalent), and bound oligomeric Aβ42 (green) was visualized using 488-conjugated streptavidin; Scale bar 10 μm; FIG. 5B shows oligomeric Aβ42 bound to Celsr3-expressing HEK293T cells (concentration showed as total peptide, monomer equivalent).

FIG. 6 shows Celsr3-Flag (red)-transfected HEK293T cells were treated with monomeric Aβ42 (200 nM total peptide), and bound monomeric Aβ42 (green) was visualized using 488-conjugated streptavidin.

FIG. 7 demonstrates that Celsr3 is a receptor for oligomeric Aβ. Particularly, FIG. 7A is a schematic of mouse Celsr3 with 9 cadherin domains, 8 EGF domains and 3 laminin domains in the extracellular domain. FIG. 7B shows Celsr3-Flag (red)-transfected or truncated Celsr3-Flag (red) were treated with oligomeric Aβ42 (200 nM total peptide, monomer equivalent), and bound oligomeric Aβ42 (green) was visualized using 488-conjugated streptavidin; Scale bar 10 μm.

FIG. 8A shows surface expression of ΔEGF/Lam_Celsr3 and Celsr3. Cell surface proteins were labeled with biotin and then precipitated with Neutravidin agarose. Precipitants and total lysates were subject to immunoblotting with the indicated antibodies.

FIG. 8B shows surface expression of Celsr3 with individual domain deletion.

FIG. 8C shows truncated Celsr3-Flag (red) transfected HEK293T cells were treated with oligomeric Aβ42 (200 nM total peptide, monomer equivalent), and bound oligomeric Aβ42 (green) was visualized using 488-conjugated streptavidin. Scale bar 10 μm.

FIG. 9 demonstrates that oligomeric Aβ competes with Frizzled3 for binding to Celsr3 and Vangl2 cKO rescues glutamatergic synapses in 5×FAD transgenic mice. Particularly, FIG. 9A shows IP assays showing interaction between Frizzled3 and Celsr3 or truncated Celsr3 that do not bind to oligomeric Aβ42 and Frizzled3 transfected in the same cell. *P<0.05. One-way ANOVA; FIG. 9B shows IP assays showing interaction between Celsr3 or truncated Celsr3 that do not bind to oligomeric Aβ42 and Vangl2 transfected in the same cell. One-way ANOVA.

FIG. 10 demonstrates that oligomeric Aβ competes with Frizzled3 for binding to Celsr3 and Vangl2 cKO rescues glutamatergic synapses in 5×FAD transgenic mice. Particularly, FIG. 10A are representative images of Bassoon (red)- and PSD95 (green)-immunoreactive puncta (arrowheads) in stratum radiatum of WT, Vangl2 cKO, 5×FAD and 5×FAD; Vangl2 cKO hippocampus (CA1). n=5 of WT mice, n=4 of Vangl2 cKO mice, n=4 of 5×FAD mice and n=8 of 5×FAD; Vangl2 cKO mice. Means±SEM. FIG. 10B is a schematic illustration of the PCP components as distributed at a neuronal synapse.

FIG. 11 demonstrates that the Wnt/Vangl2/Ryk signaling axis mediates synapse loss induced by oligomeric amyloid β. Particularly, FIG. 11A shows representative images and quantification of synaptic puncta (arrowheads) after Wnt5a and/or Ryk antibody addition to WT hippocampal neurons. n=3 experiments (n=27 neurons in IgG control, n=22 neurons in Ryk antibody, n=24 in IgG+Wnt5a, and n=20 neurons in Ryk antibody+Wnt5a), *P<0.05, **P<0.01. One-way ANOVA; Scale bar 5 μm; FIG. 11B shows representative images and quantification of synaptic puncta (arrowheads) after Wnt5a addition to Vangl2^{+/+} and Vangl2^{fl/fl} hippocampal neurons. n=3 Vangl2^{+/+} mice and n=4 Vangl2fl/fl mice. *P<0.05, **P<0.01. One-way ANOVA; Scale bar 5 μm; FIG. 11C shows representative images and quantification of synaptic puncta (arrowheads) after oligomeric Aβ and/or Ryk antibody addition to WT hippocampal neurons. n=3 experiments (n=26 neurons in IgG control, n=33 neurons in Ryk antibody, n=34 neurons in oligomeric Aβ, and n=39 neurons in Ryk antibody+oligomeric Aβ), ***P<0.001, compared to IgG control. One-way ANOVA; Scale bar 5 μm; FIG. 11D shows Mouse Ryk-HA (red) or human Ryk-Flag (Red)-transfected HEK293T cells were treated with oligomeric Aβ42 (200 nM total peptide, monomer equivalent), and bound oligomeric Aβ42 (green) was visualized using 488-conjugated streptavidin. Scale bar 10 μm.

FIG. 12 demonstrates that Ryk is involved in oligomeric amyloid beta-mediated synaptotoxicity in vivo. Particularly, shows representative images of Bassoon (red)- and PSD95 (green)-immunoreactive puncta (arrowheads) in stratum radiatum of Ryk^{+/+} and Ryk cKO hippocampus (CA1) with or without oligomeric Aβ injection. *P<0.05, One-way ANOVA. n=4 of Ryk^{+/+} mice, n=3 of Ryk^{+/+} mice with oligomeric Aβ injection, n=3 of Ryk cKO mice and n=3 of Ryk cKO mice with oligomeric Aβ injection. Means±SEM.

FIG. 13A and FIG. 13B demonstrate that deletion of Ryk on synapse number and cognitive function in a mouse model of Alzheimer's disease. Particularly, FIG. 13A shows Ryk cKO mice were crossed with 5×FAD transgenic mice. AAV-Cre was injected into the hippocampal CA1 region of 8-week-old mice for 2 months. FIG. 13B shows the objective recognition procedure.

FIG. 14A to FIG. 14D demonstrate that monoclonal Ryk antibody rescues the synapse loss in Alzheimer's disease mouse model. Particularly, FIG. 14A shows timeline outlining experimental details of the monoclonal Ryk antibody infusion. FIG. 14B is a schematic showing the implantation of cannula and minipump. FIG. 14C shows representative images of Bassoon (red)- and PSD95 (green)-immunoreactive puncta (arrowheads) in stratum radiatum. FIG. 14D shows quantification data of the presynaptic-, postsynaptic- and colocalized puncta. Means±SEM.

FIG. 15A and FIG. 15B demonstrate that Aβ oligomers bind to human-Celsr3. Particularly, FIG. 15A shows amino acid alignment of Laminin G1 domains of hCelsr3 (SEQ ID NO: 35) and mCelsr3 (SEQ ID NO: 36), alignment of EGF 7 domains of hCelsr3 (SEQ ID NO: 37) and mCelsr3 (SEQ ID NO: 38), and alignment of EGF 8 domains of hCelsr3 and mCelsr3 (SEQ ID NO: 39). FIG. 15B shows binding of Aβ42 (200 nM total peptide, monomer equivalent) with hCelsr3-Flag-transfected or truncated hCelsr3-Flag-transfected HEK203T cells. Bound oligomeric Aβ42 (green) was visualized using 488-conjugated streptavidin. Scale bar 10 μm.

### DETAILED DESCRIPTION OF THE INVENTION

Provided herein are methods and agents for modulating the signaling pathway and components thereof that are responsible for assembly and disassembly of synapses in neurons, including amyloid beta (Aβ) mediated synaptotoxicity and synapse loss. Also provided herein are methods and agents for preventing, managing and treating a disease or condition associated with Aβ-mediated synaptotoxicity and synapse loss. Also provided herein are methods for screening and identifying candidate agents capable of modulating synapse formation and (Aβ) mediated synaptotoxicity. Additional features of the present disclosure will become apparent to those skilled in the art upon consideration of the following detailed description of particular embodiments.

General Techniques

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003).

### Terminology

Unless described otherwise, all technical and scientific terms used herein have the same meaning as is commonly understood by one of ordinary skill in the art. For purposes of interpreting this specification, the following description of terms will apply and whenever appropriate, terms used in the singular will also include the plural and vice versa. All patents, applications, published applications, and other publications are incorporated by reference in their entirety. In the event that any description of terms set forth conflicts with any document incorporated herein by reference, the description of term set forth below shall control.

The singular terms “a,” “an,” and “the” as used herein include the plural reference unless the context clearly indicates otherwise.

The terms “about” and “approximately” mean within 20%, within 15%, within 10%, within 9%, within 8%, within 7%, within 6%, within 5%, within 4%, within 3%, within 2%, within 1%, or less of a given value or range.

The term “Amyloid beta,” or “Aβ” denotes the group of peptides ranging in size from 37 to 49 amino acid residues, which are produced through the proteolytic processing of the amyloid precursor protein (APP) by β-secretase and γ-secretase. Sequences of different Aβ isoforms are known in the art (Nunan et al. FEBS Lett. 2000 Oct. 13; 483(1):6-10). For example, the primary amino acid sequence of the 42-amino acid Aβ isoform (Aβ42) is DAEFRHDSGYEVHHQKLVFFAEDVGSNKGAIIGLMVGGVVIA (SEQ ID NO: 27) (Chen et al., Acta Pharmacologica Sinica volume 38, pages 1205-1235(2017)). As used herein, “Amyloid beta,” or “Aβ” can refer to either monomeric Aβ or oligomeric Aβ. The term “oligomeric Aβ” or “Aβ oligomer” denotes oligomers or aggregates formed by a group of Aβ peptides that can be either the same or different monomeric Aβ isoforms. In some embodiments, oligomeric Aβ can contain from about 2 to 20 monomeric Aβ peptides, such as about 2, about 3, about 4, about 5, about 6, about 7, about 8, about 9, about 10, about 11, about 12, about 13, about 14, about 15, about 16, about 17, about 18, about 19, or about 20 monomeric Aβ peptides. In specific embodiments, oligomeric Aβ can contain from about 2 to 4 monomeric Aβ peptides.

“Aβ induced loss of synapses” refers to the etiological phenomenon or process where the number of synapses formed in a population of neurons is reduced when the neurons are exposed to Aβ peptides (e.g., oligomeric Aβ deposited in plaques). The synapse loss may lead to progressive nervous system disorder or neurodegeneration, including the Alzheimer's disease and the Parkinson's disease.

The term “antibody,” “immunoglobulin,” or “Ig” is used interchangeably herein, and is used in the broadest sense and specifically encompasses, for example, individual monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments of antibodies, as described below. An antibody can be human, humanized, chimeric and/or affinity matured, as well as an antibody from other species, for example, mouse and rabbit, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). In specific embodiments, the specific molecular antigen can be bound by an antibody provided herein, including a Ryk polypeptide, a Ryk fragment, or a Ryk epitope. In specific embodiments, the specific molecular antigen can be bound by an antibody provided herein, including a Celsr3 polypeptide, a Celsr3 fragment, or a Celsr3 epitope. In specific embodiments, the specific molecular antigen can be bound by an antibody provided herein, including an Aβ polypeptide, an Aβ fragment, or an Aβ epitope. Antibodies also include, but are not limited to, synthetic antibodies, recombinantly produced antibodies, camelized antibodies, intrabodies, anti-idiotypic (anti-Id) antibodies, and functional fragments (e.g., antigen-binding fragments) of any of the above, which refers to a portion of an antibody heavy or light chain polypeptide that retains some or all of the binding activity of the antibody from which the fragment was derived. Non-limiting examples of functional fragments include single-chain Fvs (scFv) (e.g., including monospecific, bispecific, etc.), Fab fragments, F(ab′) fragments, F(ab)₂ fragments, F(ab′)2 fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Plückthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. An antibody against an antigen may be agonistic antibodies or antagonistic antibodies.

The terms “antigen-binding fragment,” “antigen-binding domain,” “antigen-binding region,” and similar terms refer to that portion of an antibody, which comprises the amino acid residues that interact with an antigen and confer on the binding agent its specificity and affinity for the antigen (e.g., the CDRs).

An “epitope” is the site on the surface of an antigen molecule to which a single antibody molecule binds, such as a localized region on the surface of an antigen that is capable of being bound to one or more antigen binding regions of an antibody, and that has antigenic or immunogenic activity in an animal, such as a mammal (e.g., a human), that is capable of eliciting an immune response. An epitope having immunogenic activity is a portion of a polypeptide that elicits an antibody response in an animal. An epitope having antigenic activity is a portion of a polypeptide to which an antibody binds as determined by any method well known in the art, including, for example, by an immunoassay. Antigenic epitopes need not necessarily be immunogenic. Epitopes often consist of chemically active surface groupings of molecules such as amino acids or sugar side chains and have specific three-dimensional structural characteristics as well as specific charge characteristics. Antibody epitopes may be linear epitopes or conformational epitopes. Linear epitopes are formed by a continuous sequence of amino acids in a protein. Conformational epitopes are formed of amino acids that are discontinuous in the protein sequence, but which are brought together upon folding of the protein into its three-dimensional structure. Induced epitopes are formed when the three-dimensional structure of the protein is in an altered conformation, such as following activation or binding of another protein or ligand.

The term “binds” or “binding” refer to an interaction between molecules including, for example, to form a complex (e.g. a Celsr-Frizzled complex). Interactions can be, for example, non-covalent interactions including hydrogen bonds, ionic bonds, hydrophobic interactions, and/or van der Waals interactions. A complex can also include the binding of two or more molecules (e.g. Celsr and Frizzled) held together by covalent or non-covalent bonds, interactions, or forces. The strength of the total non-covalent interactions between a single target-binding site of a binding protein (e.g., Celsr) and a single target site of a target molecule (e.g., Frizzled) is the affinity of the binding protein or functional fragment for that target site. The ratio of dissociation rate (k_{off}) to association rate (kₒₙ) of a binding protein to a monovalent target site (k_{off}/kₒₙ) is the dissociation constant K_{D}, which is inversely related to affinity. The lower the K_{D} value, the higher the affinity of the antibody. The value of K_{D} varies for different complexes of binding molecules depends on both kₒₙ and k_{off}. The dissociation constant K_{D} for a binding protein provided herein can be determined using any method provided herein or any other method well known to those skilled in the art. The affinity at one binding site does not always reflect the true strength of the interaction between a binding protein and the target molecule. When complex target molecule containing multiple, repeating target sites, such as a polyvalent target protein, come in contact with a binding molecule containing multiple target binding sites, the interaction of the binding protein with the target protein at one site will increase the probability of a reaction at a second site.

The term “binding affinity” generally refers to the strength of the sum total of noncovalent interactions between a single binding site of a molecule (e.g., a binding protein such as Celsr) and its binding partner (e.g., Frizzled). Unless indicated otherwise, as used herein, “binding affinity” refers to intrinsic binding affinity which reflects a 1:1 interaction between members of a binding pair (e.g., Celsr and Frizzled). The affinity of a binding molecule X for its binding partner Y can generally be represented by the dissociation constant (K_{D}). Affinity can be measured by common methods known in the art, including those described herein. Low-affinity binding proteins generally bind target proteins slowly and tend to dissociate readily, whereas high-affinity binding proteins generally bind target proteins faster and tend to remain bound longer. A variety of methods of measuring binding affinity are known in the art, any of which can be used for purposes of the present disclosure. Specific illustrative embodiments include the following. In one embodiment, the “K_{D}” or “K_{D} value” may be measured by assays known in the art, for example by a binding assay. The K_{D} may be measured in a RIA, for example, performed with the binding pair of Celsr and Frizzled in the presence or absence of Vangl. The K_{D} or K_{D} value may also be measured by using surface plasmon resonance assays by BIACORE®, using, for example, a BIACORE® TM-2000 or a BIACORE® TM-3000, or by biolayer interferometry using, for example, the OCTET® QK384 system. An “on-rate” or “rate of association” or “association rate” or “kₒₙ” may also be determined with the same surface plasmon resonance or biolayer interferometry techniques described above using, for example, a BIACORE® TM-2000 or a BIACORE® TM-3000, or the OCTET® QK384 system.

A molecule (e.g., an agonistic or antagonistic agent) which “binds a target molecule of interest” is one that binds the target molecule with sufficient affinity such that the molecule is useful, for example, as a diagnostic or therapeutic agent in targeting a cell or tissue expressing the target molecule, and does not significantly cross-react with other molecules. In such embodiments, the extent of binding of the molecule to a “non-target” molecule will be less than about 10% of the binding of the molecule to its particular target molecule, for example, as determined by fluorescence activated cell sorting (FACS) analysis or RIA.

The terms “antibodies that specifically bind to Celsr3,” “antibodies that specifically bind to a Celsr3 epitope,” and analogous terms are also used interchangeably herein and refer to antibodies that specifically bind to a Celsr3 polypeptide, such as a Celsr3 antigen, or fragment, or epitope (e.g., human Celsr3 such as a human Celsr3 polypeptide, antigen, or epitope). An antibody that specifically binds to Celsr3 (e.g., human Celsr3) may bind to the extracellular domain or a peptide derived from the extracellular domain of Celsr3. An antibody that specifically binds to a Celsr3 antigen (e.g., human Celsr3) may be cross-reactive with related antigens (e.g., cynomolgus Celsr3). In certain embodiments, an antibody that specifically binds to a Celsr3 antigen does not cross-react with other antigens. An antibody that specifically binds to a Celsr3 antigen can be identified, for example, by immunoassays, BIACORE®, or other techniques known to those of skill in the art. An antibody binds specifically to a Celsr3 antigen when it binds to a Celsr3 antigen with higher affinity than to any cross-reactive antigen as determined using experimental techniques, such as radioimmunoassays (RIA) and enzyme linked immunosorbent assays (ELISAs). The term “anti-Celsr3 antibody” or “an antibody that binds to Celsr3” includes an antibody that is capable of binding Celsr3 with sufficient affinity such that the antibody is useful, for example, as a diagnostic agent in targeting Celsr3. In various embodiments, anti-Celsr3 antibody binds to an epitope of Celsr3 that is conserved among Celsr3 from different species (e.g., between human and cynomolgus Celsr3).

The terms “antibodies that specifically bind to Ryk,” “antibodies that specifically bind to a Ryk epitope,” and analogous terms are also used interchangeably herein and refer to antibodies that specifically bind to a Ryk polypeptide, such as a Ryk antigen, or fragment, or epitope (e.g., human Ryk such as a human Ryk polypeptide, antigen, or epitope). An antibody that specifically binds to Ryk (e.g., human Ryk) may bind to the extracellular domain or a peptide derived from the extracellular domain of Ryk. An antibody that specifically binds to a Ryk antigen (e.g., human Ryk) may be cross-reactive with related antigens (e.g., cynomolgus Ryk). In certain embodiments, an antibody that specifically binds to a Ryk antigen does not cross-react with other antigens. An antibody that specifically binds to a Ryk antigen can be identified, for example, by immunoassays, BIACORE®, or other techniques known to those of skill in the art. An antibody binds specifically to a Ryk antigen when it binds to a Ryk antigen with higher affinity than to any cross-reactive antigen as determined using experimental techniques, such as radioimmunoassays (RIA) and enzyme linked immunosorbent assays (ELISAs). The term “anti-Ryk antibody” or “an antibody that binds to Ryk” includes an antibody that is capable of binding Ryk with sufficient affinity such that the antibody is useful, for example, as a diagnostic agent in targeting Ryk. In certain embodiments, anti-Ryk antibody binds to an epitope of Ryk that is conserved among Ryk from different species (e.g., between human and cynomolgus Ryk).

The terms “antibodies that specifically bind to Aβ,” “antibodies that specifically bind to an Aβ epitope,” and analogous terms are also used interchangeably herein and refer to antibodies that specifically bind to an Aβ polypeptide (such as an Aβ antigen, or fragment, or epitope) either in the monomeric form or forming part of an oligomeric Aβ complex or aggregate. An antibody that specifically binds to an Aβ antigen can be identified, for example, by immunoassays, BIACORE®, or other techniques known to those of skill in the art. An antibody binds specifically to an Aβ antigen when it binds to an Aβ antigen with higher affinity than to any cross-reactive antigen as determined using experimental techniques, such as radioimmunoassays (RIA) and enzyme linked immunosorbent assays (ELISAs). The term “anti-Aβ antibody” or “an antibody that binds to Aβ” includes an antibody that is capable of binding Aβ with sufficient affinity such that the antibody is useful, for example, as a diagnostic agent in targeting Aβ.

Typically, a specific or selective reaction will be at least twice background signal or noise and may be more than 10 times background. See, e.g., Fundamental Immunology 332-36 (Paul ed., 2d ed. 1989) for a discussion regarding antibody specificity. An antibody which “binds an antigen of interest” (e.g., a target antigen such as Celsr3, Ryk, or Aβ) is one that binds the antigen with sufficient affinity such that the antibody is useful as a therapeutic agent in targeting a cell or tissue expressing the antigen, and does not significantly cross-react with other proteins. In such embodiments, the extent of binding of the antibody to a “non-target” protein will be less than about 10% of the binding of the antibody to its particular target protein, for example, as determined by fluorescence activated cell sorting (FACS) analysis or RIA.

With regard to the binding of an antibody to a target molecule (e.g., Celsr3, Ryk, or Aβ), the term “specific binding,” “specifically binds to,” or “is specific for” a particular polypeptide or an epitope on a particular polypeptide target means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding,” “specifically binds to,” or “is specific for” a particular polypeptide or an epitope on a particular polypeptide target as used herein refers to binding where a molecule binds to a particular polypeptide or epitope on a particular polypeptide without substantially binding to any other polypeptide or polypeptide epitope. In certain embodiments, an antibody that binds to Celsr3 has a dissociation constant (K_{D}) of less than or equal to 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, an antibody that binds to Ryk has a dissociation constant (K_{D}) of less than or equal to 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, an antibody that binds to Aβ has a dissociation constant (K_{D}) of less than or equal to 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM.

With regard to the binding of an antibody to a target molecule, the term “preferential binding” or “preferentially binds to” a particular polypeptide or an fragment on a particular target molecule with respect to a reference molecule means binding of the target molecule is measurably higher than binding of the reference molecule, while the reference molecule may or may not also bind to the antibody. For example, in some embodiments, an antibody preferentially binds to Celsr3 (such as a human Celsr3 polypeptide, antigen, or epitope) over Celsr2 (such as a human Celsr2 polypeptide, antigen, or epitope). Preferential binding can be determined, for example, by determining the binding affinity. For example, antibody that preferentially binds to a target molecule (such as the molecule, or an antigen or epitope thereof) over a reference molecule (such as the molecule, or an antigen or epitope thereof) can bind to the target molecule with a K_{D} less than the K_{D} exhibited relative to the reference molecule. In some embodiments, the antibody preferentially binds a target molecule with a K_{D} less than half of the K_{D} exhibited relative to the reference molecule. In some embodiments, the antibody preferentially binds a target molecule with a K_{D} at least 10 times less than the K_{D} exhibited relative to the reference molecule. In some embodiments, the antibody preferentially binds a target molecule with a K_{D} with K_{D} that is about 75%, about 50%, about 25%, about 10%, about 5%, about 2.5%, or about 1% of the K_{D} exhibited relative to the reference molecule. In some embodiments, the ratio between the K_{D} exhibited by the antibody when binding to the reference molecule and the K_{D} exhibited when binding to the target molecule is at least 2 fold, at least 3 fold, at least 4 fold, at least 5 fold, at least 10 fold, at least 20 fold, at least 100 fold, at least 500 fold, at least 103 fold, at least 10⁴ fold, or at least 105 fold. An antibody that preferentially binds to a target molecule can be identified, for example, by immunoassays (e.g., ELISA, fluorescent immunosorbent assay, chemiluminescence immune assay, radioimmunoassay (RIA), enzyme multiplied immunoassay, solid phase radioimmunoassay (SPRIA), a surface plasmon resonance (SPR) assay (e.g., BIACORE®), a fluorescence polarization assay, a fluorescence resonance energy transfer (FRET) assay, Dot-blot assay, fluorescence activated cell sorting (FACS) assay, or other techniques known to those of skill in the art.

The preferential binding can also be determined by binding assays and be indicated by, for example, fluorescence intensity (“MFI”). For example, an antibody or antigen binding fragment that preferentially binds to the Celsr 3 over Celsr 2 can bind to Celsr 3 with an MFI that is higher than the MFI as exhibited relative to Celsr 2. In various embodiments, the antibody or antigen binding fragment binds to Celsr 3 with an MFI that is at least twice as high as the MFI as exhibited relative to Celsr 2. In various embodiments, antibody or the antigen binding fragment binds to Celsr 3 with an MFI that is at least three times as high as the MFI as exhibited relative to Celsr 2. In various embodiments, antibody or the antigen binding fragment binds to Celsr 3 with an MFI that is at least five times, at least ten times, at least fifteen times, or at least twenty times as high as the MFI as exhibited relative to Celsr 2.

The term “compete” when used in the context of two or more molecules that compete for binding to the same target molecule (e.g., an Aβ inhibitor competes with Aβ for binding to Celsr), means competition as determined by an assay in which the binding molecule under study (e.g., a candidate anti-Celsr antibody) prevents or inhibits the specific binding of a reference molecule (e.g., Aβ) to a common target molecule (e.g., Celsr). Numerous types of competitive binding assays can be used to determine if a test agent competes with a reference ligand for binding to a target molecule. Examples of assays that can be employed include solid phase direct or indirect RIA, solid phase direct or indirect enzyme immunoassay (EIA), sandwich competition assay (see, e.g., Stahli et al., 1983, Methods in Enzymology 9:242-53), solid phase direct biotin-avidin EIA (see, e.g., Kirkland et al., 1986, J. Immunol. 137:3614-19), solid phase direct labeled assay, solid phase direct labeled sandwich assay (see, e.g., Harlow and Lane, Antibodies, A Laboratory Manual (1988)), solid phase direct label RIA using 1-125 label (see, e.g., Morel et al., 1988, Mol. Immunol. 25:7-15), and direct labeled RIA (Moldenhauer et al., 1990, Scand. J. Immunol. 32:77-82). Typically, such an assay involves the use of a purified target molecule bound to a solid surface, or cells bearing either of an unlabeled test target-binding lasso peptide or a labeled reference target-binding protein (e.g., reference target-binding ligand). Competitive inhibition may be measured by determining the amount of label bound to the solid surface in the presence of the test target-binding lasso peptide. Usually the test target-binding protein is present in excess. Target-binding molecules identified by competition assay include binding molecules that bind to the same target site as the reference and binding molecules that to an adjacent target site sufficiently proximal to the target site bound by the reference for steric hindrance to occur. Additional details regarding methods for determining competitive binding are described herein. Usually, when a competing binding molecule is present in excess, it will inhibit specific binding of a reference to a common target molecule by at least 30%, for example 40%, 45%, 50%, 55%, 60%, 65%, 70%, or 75%. In some instances, binding is inhibited by at least 80%, 85%, 90%, 95%, 96%, 97%, 98%, 99%, or more.

The terms “blocking” agent, “antagonist,” and “inhibitor” of a target molecule are used interchangeably herein to refer to an agent that reduces or inhibits a biological effect induced by the target molecule, e.g., in vivo or in vitro. The agent can be a small molecule compound or a biological molecule such as a nucleic acid or polypeptide. In assessing the strength of inhibition, the biological effect can be measured in the presence and absence of the candidate agent. In certain embodiments, the biological effect measured in the presence of the antagonist is equal to or less than 10%, 15%, 20%, 25%, 30%, 35%, 40%, 45%, 50%, 55%, 60%, 65%, 70%, 75%, 80%, 85%, 90%, 95%, or 98% of the same biological effect measured in the absence of the antagonist.

An “agonist” of a target molecule refers to an agent that increases or enhances a biological effect induced by the target molecule, e.g., in vivo or in vitro. The agent can be a small molecule compound or a biological molecule such as a nucleic acid or polypeptide. In assessing the level of enhancement, the biological effect can be measured in the presence and absence of the candidate agent. In certain embodiments, the biological effect measured in the absence of the agonist is equal to or less than 10%, 15%, 20%, 25%, 30%, 35%, 40%, 45%, 50%, 55%, 60%, 65%, 70%, 75%, 80%, 85%, 90%, 95%, or 98% of the same biological effect measured in the presence of the agonist. For example, an agonist of Celsr3 as described herein can be a molecule that is capable of activating or otherwise increasing one or more of the biological activities of Celsr3, such as in a cell expressing Celsr3. In some embodiments, an agonist of Celsr3 (e.g., an agonistic antibody as described herein) may, for example, act by activating or otherwise increasing the activation and/or cell signaling pathways of a cell expressing a Celsr3 protein, thereby increasing a Celsr3-mediated biological activity of the cell relative to the Celsr3-mediated biological activity in the absence of agonist. In some embodiments, the cell expressing a Celsr-3 protein is a neuron, and the Celsr-3 mediated biological activity is Celsr-3 mediated formation of neuronal synapses.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue.

A “functional Fc region” possesses an “effector function” of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays as disclosed.

A “native sequence Fc region” comprises an amino acid sequence identical to the amino acid sequence of an Fc region found in nature, and not manipulated, modified, and/or changed (e.g., isolated, purified, selected, including or combining with other sequences such as variable region sequences) by a human. Native sequence human IgG1 Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof. For example, a native human IgG1 Fc region amino acid sequence is as follows:

(SEQ ID NO: 40)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK.

An exemplary native human IgG4 Fc region sequence is as follows:

(SEQ ID NO: 41)
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPSCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSPGK.

A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

The term “native” when used in connection with biological materials such as nucleic acid molecules, polypeptides, host cells, and the like, refers to those which are found in nature and not manipulated, modified, and/or changed (e.g., isolated, purified, selected) by a human being.

The term “variant” when used in relation to a protein or peptide may refer to a peptide or polypeptide comprising one or more (such as, for example, about 1 to about 25, about 1 to about 20, about 1 to about 15, about 1 to about 10, or about 1 to about 5) amino acid sequence substitutions, deletions, and/or additions as compared to a native or unmodified sequence. For example, a variant of Celsr3 EGF7 domain may result from one or more (such as, for example, about 1 to about 25, about 1 to about 20, about 1 to about 15, about 1 to about 10, or about 1 to about 5) changes to an amino acid sequence of a native Celsr3 EGF7 domain sequence. Variants may be naturally occurring, such as allelic or splice variants, or may be artificially constructed. Polypeptide variants may be prepared from the corresponding nucleic acid molecules encoding the variants. In specific embodiments, the variant of a protein or peptide retains functional activity of the native protein or peptide. In certain embodiments, the variant is encoded by a single nucleotide polymorphism (SNP) variant of a nucleic acid molecule that encodes the native protein or peptide. A functional variant of a peptide refers to a variant of the peptide that retains at least one function or activity of interest of the native peptide. For example, a functional variant of Celsr3 Laminin G1 can have about 95% sequence identity of the native Celsr3 Laminin G1 domain sequence and retains the functionality of forming intercellular complexes across the synaptic cleft. For example, a functional variant of Celsr3 EGF7 domain can have about 90% sequence identity of the native Celsr3 EGF7 domain sequence and retain the activity of binding with Aβ.

The Celsr family proteins are adhesion G protein-coupled receptors. In humans, at least three Celsr proteins, Celsr1, Celsr2 and Celsr3, belong to this family. As used herein, the term “Cadherin EGF LAG Seven-Pass G-Type Receptor,” “CELSR,” “Celsr,” “Protein Celsr,” or “Celsr polypeptide,” encompasses a polypeptide (“polypeptide” and “protein” are used interchangeably herein), including any native polypeptide, from any vertebrate source, including mammals such as primates (e.g., humans and cynomolgus monkeys (cynomolgus)), dogs, and rodents (e.g., mice and rats), unless otherwise indicated. In certain embodiments, the terms include “related Celsr polypeptides,” including SNP variants thereof. The term “Celsr” also encompasses “full-length,” unprocessed Celsr as well as any form of Celsr that results from processing in the cell.

In some embodiments, the Celsr1 has an amino acid sequence of:

(SEQ ID NO: 28)
MAPPPPPVLPVLLLLAAAAALPAMGLRAAAWEPRVPGGTRAFALRPGCTYAVGAA
CTPRAPRELLDVGRDGRLAGRRRVSGAGRPLPLQVRLVARSAPTALSRRLRARTHLP
GCGARARLCGTGARLCGALCFPVPGGCAAAQHSALAAPTTLPACRCPPRPRPRCPGR
PICLPPGGSVRLRLLCALRRAAGAVRVGLALEAATAGTPSASPSPSPPLPPNLPEARA
GPARRARRGTSGRGSLKFPMPNYQVALFENEPAGTLILQLHAHYTIEGEEERVSYYM
EGLFDERSRGYFRIDSATGAVSTDSVLDRETKETHVLRVKAVDYSTPPRSATTYITVL
VKDTNDHSPVFEQSEYRERVRENLEVGYEVLTIRASDRDSPINANLRYRVLGGAWD
VFQLNESSGVVSTRAVLDREEAAEYQLLVEANDQGRNPGPLSATATVYIEVEDEND
NYPQFSEQNYVVQVPEDVGLNTAVLRVQATDRDQGQNAAIHYSILSGNVAGQFYLH
SLSGILDVINPLDFEDVQKYSLSIKAQDGGRPPLINSSGVVSVQVLDVNDNEPIFVSSP
FQATVLENVPLGYPVVHIQAVDADSGENARLHYRLVDTASTFLGGGSAGPKNPAPT
PDFPFQIHNSSGWITVCAELDREEVEHYSFGVEAVDHGSPPMSSSTSVSITVLDVNDN
DPVFTQPTYELRLNEDAAVGSSVLTLQARDRDANSVITYQLTGGNTRNRFALSSQRG
GGLITLALPLDYKQEQQYVLAVTASDGTRSHTAHVLINVTDANTHRPVFQSSHYTVS
VSEDRPVGTSIATLSANDEDTGENARITYVIQDPVPQFRIDPDSGTMYTMMELDYEN
QVAYTLTIMAQDNGIPQKSDTTTLEILILDANDNAPQFLWDFYQGSIFEDAPPSTSILQ
VSATDRDSGPNGRLLYTFQGGDDGDGDFYIEPTSGVIRTQRRLDRENVAVYNLWAL
AVDRGSPTPLSASVEIQVTILDINDNAPMFEKDELELFVEENNPVGSVVAKIRANDPD
EGPNAQIMYQIVEGDMRHFFQLDLLNGDLRAMVELDFEVRREYVLVVQATSAPLVS
RATVHILLVDQNDNPPVLPDFQILFNNYVTNKSNSFPTGVIGCIPAHDPDVSDSLNYT
FVQGNELRLLLLDPATGELQLSRDLDNNRPLEALMEVSVSDGIHSVTAFCTLRVTIIT
DDMLTNSITVRLENMSQEKFLSPLLALFVEGVAAVLSTTKDDVFVFNVQNDTDVSSN
ILNVTFSALLPGGVRGQFFPSEDLQEQIYLNRTLLTTISTQRVLPFDDNICLREPCENY
MKCVSVLRFDSSAPFLSSTTVLFRPIHPINGLRCRCPPGFTGDYCETEIDLCYSDPCGA
NGRCRSREGGYTCECFEDFTGEHCEVDARSGRCANGVCKNGGTCVNLLIGGFHCVC
PPGEYERPYCEVTTRSFPPQSFVTFRGLRQRFHFTISLTFATQERNGLLLYNGRFNEKH
DFIALEIVDEQVQLTFSAGETTTTVAPKVPSGVSDGRWHSVQVQYYNKPNIGHLGLP
HGPSGEKMAVVTVDDCDTTMAVRFGKDIGNYSCAAQGTQTGSKKSLDLTGPLLLG
GVPNLPEDFPVHNRQFVGCMRNLSVDGKNVDMAGFIANNGTREGCAARRNFCDGR
RCQNGGTCVNRWNMYLCECPLRFGGKNCEQAMPHPQLFSGESVVSWSDLNIIISVP
WYLGLMFRTRKEDSVLMEATSGGPTSFRLQILNNYLQFEVSHGPSDVESVMLSGLR
VTDGEWHHLLIELKNVKEDSEMKHLVTMTLDYGMDQNKADIGGMLPGLTVRSVVV
GGASEDKVSVRRGFRGCMQGVRMGGTPTNVATLNMNNALKVRVKDGCDVDDPCT
SSPCPPNSRCHDAWEDYSCVCDKGYLGINCVDACHLNPCENMGACVRSPGSPQGYV
CECGPSHYGPYCENKLDLPCPRGWWGNPVCGPCHCAVSKGFDPDCNKTNGQCQCK
ENYYKLLAQDTCLPCDCFPHGSHSRTCDMATGQCACKPGVIGRQCNRCDNPFAEVT
TLGCEVIYNGCPKAFEAGIWWPQTKFGQPAAVPCPKGSVGNAVRHCSGEKGWLPPE
LFNCTTISFVDLRAMNEKLSRNETQVDGARALQLVRALRSATQHTGTLFGNDVRTA
YQLLGHVLQHESWQQGFDLAATQDADFHEDVIHSGSALLAPATRAAWEQIQRSEGG
TAQLLRRLEGYFSNVARNVRRTYLRPFVIVTANMILAVDIFDKFNFTGARVPRFDTIH
EEFPRELESSVSFPADFFRPPEEKEGPLLRPAGRRTTPQTTRPGPGTEREAPISRRRRHP
DDAGQFAVALVIIYRTLGQLLPERYDPDRRSLRLPHRPIINTPMVSTLVYSEGAPLPRP
LERPVLVEFALLEVEERTKPVCVFWNHSLAVGGTGGWSARGCELLSRNRTHVACQC
SHTASFAVLMDISRRENGEVLPLKIVTYAAVSLSLAALLVAFVLLSLVRMLRSNLHSI
HKHLAVALFLSQLVFVIGINQTENPFLCTVVAILLHYIYMSTFAWTLVESLHVYRMLT
EVRNIDTGPMRFYYVVGWGIPAIVTGLAVGLDPQGYGNPDFCWLSLQDTLIWSFAG
PIGAVIIINTVTSVLSAKVSCQRKHHYYGKKGIVSLLRTAFLLLLLISATWLLGLLAVN
RDALSFHYLFAIFSGLQGPFVLLFHCVLNQEVRKHLKGVLGGRKLHLEDSATTRATL
LTRSLNCNTTFGDGPDMLRTDLGESTASLDSIVRDEGIQKLGVSSGLVRGSHGEPDAS
LMPRSCKDPPGHDSDSDSELSLDEQSSSYASSHSSDSEDDGVGAEEKWDPARGAVHS
TPKGDAVANHVPAGWPDQSLAESDSEDPSGKPRLKVETKVSVELHREEQGSHRGEY
PPDQESGGAARLASSQPPEQRKGILKNKVTYPPPLTLTEQTLKGRLREKLADCEQSPT
SSRTSSLGSGGPDCAITVKSPGREPGRDHLNGVAMNVRTGSAQADGSDSEKP

GenBank™ accession number NM_001378328 provides another exemplary human Celsr1 nucleic acid sequence.

In some embodiments, the Celsr2 has an amino acid sequence of:

(SEQ ID NO: 29)
MRSPATGVPLPTPPPPLLLLLLLLLPPPLLGDQVGPCRSLGSRGRGSSGACAPMGWLC
PSSASNLWLYTSRCRDAGTELTGHLVPHHDGLRVWCPESEAHIPLPPAPEGCPWSCR
LLGIGGHLSPQGKLTLPEEHPCLKAPRLRCQSCKLAQAPGLRAGERSPEESLGGRRKR
NVNTAPQFQPPSYQATVPENQPAGTPVASLRAIDPDEGEAGRLEYTMDALFDSRSNQ
FFSLDPVTGAVTTAEELDRETKSTHVFRVTAQDHGMPRRSALATLTILVTDTNDHDP
VFEQQEYKESLRENLEVGYEVLTVRATDGDAPPNANILYRLLEGSGGSPSEVFEIDPR
SGVIRTRGPVDREEVESYQLTVEASDQGRDPGPRSTTAAVFLSVEDDNDNAPQFSEK
RYVVQVREDVTPGAPVLRVTASDRDKGSNAVVHYSIMSGNARGQFYLDAQTGALD
VVSPLDYETTKEYTLRVRAQDGGRPPLSNVSGLVTVQVLDINDNAPIFVSTPFQATVL
ESVPLGYLVLHVQAIDADAGDNARLEYRLAGVGHDFPFTINNGTGWISVAAELDRE
EVDFYSFGVEARDHGTPALTASASVSVTVLDVNDNNPTFTQPEYTVRLNEDAAVGT
SVVTVSAVDRDAHSVITYQITSGNTRNRFSITSQSGGGLVSLALPLDYKLERQYVLAV
TASDGTRQDTAQIVVNVTDANTHRPVFQSSHYTVNVNEDRPAGTTVVLISATDEDT
GENARITYFMEDSIPQFRIDADTGAVTTQAELDYEDQVSYTLAITARDNGIPQKSDTT
YLEILVNDVNDNAPQFLRDSYQGSVYEDVPPFTSVLQISATDRDSGLNGRVFYTFQG
GDDGDGDFIVESTSGIVRTLRRLDRENVAQYVLRAYAVDKGMPPARTPMEVTVTVL
DVNDNPPVFEQDEFDVFVEENSPIGLAVARVTATDPDEGTNAQIMYQIVEGNIPEVFQ
LDIFSGELTALVDLDYEDRPEYVLVIQATSAPLVSRATVHVRLLDRNDNPPVLGNFEI
LFNNYVTNRSSSFPGGAIGRVPAHDPDISDSLTYSFERGNELSLVLLNASTGELKLSRA
LDNNRPLEAIMSVLVSDGVHSVTAQCALRVTIITDEMLTHSITLRLEDMSPERFLSPLL
GLFIQAVAATLATPPDHVVVFNVQRDTDAPGGHILNVSLSVGQPPGPGGGPPFLPSED
LQERLYLNRSLLTAISAQRVLPFDDNICLREPCENYMRCVSVLRFDSSAPFIASSSVLF
RPIHPVGGLRCRCPPGFTGDYCETEVDLCYSRPCGPHGRCRSREGGYTCLCRDGYTG
EHCEVSARSGRCTPGVCKNGGTCVNLLVGGFKCDCPSGDFEKPYCQVTTRSFPAHSF
ITFRGLRQRFHFTLALSFATKERDGLLLYNGRFNEKHDFVALEVIQEQVQLTFSAGES
TTTVSPFVPGGVSDGQWHTVQLKYYNKPLLGQTGLPQGPSEQKVAVVTVDGCDTG
VALRFGSVLGNYSCAAQGTQGGSKKSLDLTGPLLLGGVPDLPESFPVRMRQFVGCM
RNLQVDSRHIDMADFIANNGTVPGCPAKKNVCDSNTCHNGGTCVNQWDAFSCECP
LGFGGKSCAQEMANPQHFLGSSLVAWHGLSLPISQPWYLSLMFRTRQADGVLLQAI
TRGRSTITLQLREGHVMLSVEGTGLQASSLRLEPGRANDGDWHHAQLALGASGGPG
HAILSFDYGQQRAEGNLGPRLHGLHLSNITVGGIPGPAGGVARGFRGCLQGVRVSDT
PEGVNSLDPSHGESINVEQGCSLPDPCDSNPCPANSYCSNDWDSYSCSCDPGYYGDN
CTNVCDLNPCEHQSVCTRKPSAPHGYTCECPPNYLGPYCETRIDQPCPRGWWGHPT
CGPCNCDVSKGFDPDCNKTSGECHCKENHYRPPGSPTCLLCDCYPTGSLSRVCDPED
GQCPCKPGVIGRQCDRCDNPFAEVTTNGCEVNYDSCPRAIEAGIWWPRTRFGLPAAA
PCPKGSFGTAVRHCDEHRGWLPPNLFNCTSITFSELKGFAERLQRNESGLDSGRSQQL
ALLLRNATQHTAGYFGSDVKVAYQLATRLLAHESTQRGFGLSATQDVHFTENLLRV
GSALLDTANKRHWELIQQTEGGTAWLLQHYEAYASALAQNMRHTYLSPFTIVTPNI
VISVVRLDKGNFAGAKLPRYEALRGEQPPDLETTVILPESVFRETPPVVRPAGPGEAQ
EPEELARRQRRHPELSQGEAVASVIIYRTLAGLLPHNYDPDKRSLRVPKRPIINTPVVS
ISVHDDEELLPRALDKPVTVQFRLLETEERTKPICVFWNHSILVSGTGGWSARGCEVV
FRNESHVSCQCNHMTSFAVLMDVSRRENGEILPLKTLTYVALGVTLAALLLTFFFLT
LLRILRSNQHGIRRNLTAALGLAQLVFLLGINQADLPFACTVIAILLHFLYLCTFSWAL
LEALHLYRALTEVRDVNTGPMRFYYMLGWGVPAFITGLAVGLDPEGYGNPDFCWL
SIYDTLIWSFAGPVAFAVSMSVFLYILAARASCAAQRQGFEKKGPVSGLQPSFAVLLL
LSATWLLALLSVNSDTLLFHYLFATCNCIQGPFIFLSYVVLSKEVRKALKLACSRKPS
PDPALTTKSTLTSSYNCPSPYADGRLYQPYGDSAGSLHSTSRSGKSQPSYIPFLLREES
ALNPGQGPPGLGDPGSLFLEGQDQQHDPDTDSDSDLSLEDDQSGSYASTHSSDSEEE
EEEEEEEAAFPGEQGWDSLLGPGAERLPLHSTPKDGGPGPGKAPWPGDFGTTAKESS
GNGAPEERLRENGDALSREGSLGPLPGSSAQPHKGILKKKCLPTISEKSSLLRLPLEQC
TGSSRGSSASEGSRGGPPPRPPPRQSLQEQLNGVMPIAMSIKAGTVDEDSSGSEFLFFN
FLH.

GenBank™ accession number NM_001408 provides another exemplary human Celsr2 nucleic acid sequence.

In some embodiments, the Celsr3 has an amino acid sequence of:

(SEQ ID NO: 30)
MMARRPPWRGLGGRSTPILLLLLLSLFPLSQEELGGGGHQGWDPGLAATTGPRAHIG
GGALALCPESSGVREDGGPGLGVREPIFVGLRGRRQSARNSRGPPEQPNEELGIEHGV
QPLGSRERETGQGPGSVLYWRPEVSSCGRTGPLQRGSLSPGALSSGVPGSGNSSPLPS
DFLIRHHGPKPVSSQRNAGTGSRKRVGTARCCGELWATGSKGQGERATTSGAERTA
PRRNCLPGASGSGPELDSAPRTARTAPASGSAPRESRTAPEPAPKRMRSRGLFRCRFL
PQRPGPRPPGLPARPEARKVTSANRARFRRAANRHPQFPQYNYQTLVPENEAAGTA
VLRVVAQDPDAGEAGRLVYSLAALMNSRSLELFSIDPQSGLIRTAAALDRESMERHY
LRVTAQDHGSPRLSATTMVAVTVADRNDHSPVFEQAQYRETLRENVEEGYPILQLR
ATDGDAPPNANLRYRFVGPPAARAAAAAAFEIDPRSGLISTSGRVDREHMESYELVV
EASDQGQEPGPRSATVRVHITVLDENDNAPQFSEKRYVAQVREDVRPHTVVLRVTA
TDRDKDANGLVHYNIISGNSRGHFAIDSLTGEIQVVAPLDFEAEREYALRIRAQDAGR
PPLSNNTGLASIQVVDINDHIPIFVSTPFQVSVLENAPLGHSVIHIQAVDADHGENARL
EYSLTGVAPDTPFVINSATGWVSVSGPLDRESVEHYFFGVEARDHGSPPLSASASVTV
TVLDVNDNRPEFTMKEYHLRLNEDAAVGTSVVSVTAVDRDANSAISYQITGGNTRN
RFAISTQGGVGLVTLALPLDYKQERYFKLVLTASDRALHDHCYVHINITDANTHRPV
FQSAHYSVSVNEDRPMGSTIVVISASDDDVGENARITYLLEDNLPQFRIDADSGAITL
QAPLDYEDQVTYTLAITARDNGIPQKADTTYVEVMVNDVNDNAPQFVASHYTGLVS
EDAPPFTSVLQISATDRDAHANGRVQYTFQNGEDGDGDFTIEPTSGIVRTVRRLDREA
VSVYELTAYAVDRGVPPLRTPVSIQVMVQDVNDNAPVFPAEEFEVRVKENSIVGSVV
AQITAVDPDEGPNAHIMYQIVEGNIPELFQMDIFSGELTALIDLDYEARQEYVIVVQA
TSAPLVSRATVHVRLVDQNDNSPVLNNFQILFNNYVSNRSDTFPSGIIGRIPAYDPDVS
DHLFYSFERGNELQLLVVNQTSGELRLSRKLDNNRPLVASMLVTVTDGLHSVTAQC
VLRVVIITEELLANSLTVRLENMWQERFLSPLLGRFLEGVAAVLATPAEDVFIFNIQN
DTDVGGTVLNVSFSALAPRGAGAGAAGPWFSSEELQEQLYVRRAALAARSLLDVLP
FDDNVCLREPCENYMKCVSVLRFDSSAPFLASASTLFRPIQPIAGLRCRCPPGFTGDFC
ETELDLCYSNPCRNGGACARREGGYTCVCRPRFTGEDCELDTEAGRCVPGVCRNGG
TCTDAPNGGFRCQCPAGGAFEGPRCEVAARSFPPSSFVMFRGLRQRFHLTLSLSFATV
QQSGLLFYNGRLNEKHDFLALELVAGQVRLTYSTGESNTVVSPTVPGGLSDGQWHT
VHLRYYNKPRTDALGGAQGPSKDKVAVLSVDDCDVAVALQFGAEIGNYSCAAAGV
QTSSKKSLDLTGPLLLGGVPNLPENFPVSHKDFIGCMRDLHIDGRRVDMAAFVANNG
TMAGCQAKLHFCDSGPCKNSGFCSERWGSFSCDCPVGFGGKDCQLTMAHPHHFRG
NGTLSWNFGSDMAVSVPWYLGLAFRTRATQGVLMQVQAGPHSTLLCQLDRGLLSV
TVTRGSGRASHLLLDQVTVSDGRWHDLRLELQEEPGGRRGHHVLMVSLDFSLFQDT
MAVGSELQGLKVKQLHVGGLPPGSAEEAPQGLVGCIQGVWLGSTPSGSPALLPPSHR
VNAEPGCVVTNACASGPCPPHADCRDLWQTFSCTCQPGYYGPGCVDACLLNPCQN
QGSCRHLPGAPHGYTCDCVGGYFGHHCEHRMDQQCPRGWWGSPTCGPCNCDVHK
GFDPNCNKTNGQCHCKEFHYRPRGSDSCLPCDCYPVGSTSRSCAPHSGQCPCRPGAL
GRQCNSCDSPFAEVTASGCRVLYDACPKSLRSGVWWPQTKFGVLATVPCPRGALGA
AVRLCDEAQGWLEPDLFNCTSPAFRELSLLLDGLELNKTALDTMEAKKLAQRLREV
TGHTDHYFSQDVRVTARLLAHLLAFESHQQGFGLTATQDAHFNENLLWAGSALLAP
ETGDLWAALGQRAPGGSPGSAGLVRHLEEYAATLARNMELTYLNPMGLVTPNIMLS
IDRMEHPSSPRGARRYPRYHSNLFRGQDAWDPHTHVLLPSQSPRPSPSEVLPTSSSIEN
STTSSVVPPPAPPEPEPGISIIILLVYRTLGGLLPAQFQAERRGARLPQNPVMNSPVVSV
AVFHGRNFLRGILESPISLEFRLLQTANRSKAICVQWDPPGLAEQHGVWTARDCELV
HRNGSHARCRCSRTGTFGVLMDASPRERLEGDLELLAVFTHVVVAVSVAALVLTAA
ILLSLRSLKSNVRGIHANVAAALGVAELLFLLGIHRTHNQLVCTAVAILLHYFFLSTF
AWLFVQGLHLYRMQVEPRNVDRGAMRFYHALGWGVPAVLLGLAVGLDPEGYGNP
DFCWISVHEPLIWSFAGPVVLVIVMNGTMFLLAARTSCSTGQREAKKTSALTLRSSFL
LLLLVSASWLFGLLAVNHSILAFHYLHAGLCGLQGLAVLLLFCVLNADARAAWMPA
CLGRKAAPEEARPAPGLGPGAYNNTALFEESGLIRITLGASTVSSVSSARSGRTQDQD
SQRGRSYLRDNVLVRHGSAADHTDHSLQAHAGPTDLDVAMFHRDAGADSDSDSDL
SLEEERSLSIPSSESEDNGRTRGRFQRPLCRAAQSERLLTHPKDVDGNDLLSYWPALG
ECEAAPCALQTWGSERRLGLDTSKDAANNNQPDPALTSGDETSLGRAQRQRKGILK
NRLQYPLVPQTRGAPELSWCRAATLGHRAVPAASYGRIYAGGGTGSLSQPASRYSSR
EQLDLLLRRQLSRERLEEAPAPVLRPLSRPGSQECMDAAPGRLEPKDRGSTLPRRQPP
RDYPGAMAGRFGSRDALDLGAPREWLSTLPPPRRTRDLDPQPPPLPLSPQRQLSRDP
LLPSRPLDSLSRSSNSREQLDQVPSRHPSREALGPLPQLLRAREDSVSGPSHGPSTEQL
DILSSILASFNSSALSSVQSSSTPLGPHTTATPSATASVLGPSTPRSATSHSISELSPDSEV
PRSEGHS.

GenBank™ accession number NM_001407 provides another exemplary human Celsr3 nucleic acid sequence.

The Frizzled family proteins are G protein-coupled receptor proteins that can serve as receptors in the planer cell polarity (PCP) signaling pathway, the Wnt signaling pathway and/or other signaling pathways. As used herein, the term “Frizzled” encompasses a polypeptide (“polypeptide” and “protein” are used interchangeably herein), including any native polypeptide, from any vertebrate source, including mammals such as primates (e.g., humans and cynomolgus monkeys (cynomolgus)), dogs, and rodents (e.g., mice and rats), unless otherwise indicated. In certain embodiments, the terms include “related Frizzled polypeptides,” including SNP variants thereof. The term “Frizzled” also encompasses “full-length,” unprocessed Frizzled as well as any form of Frizzled that results from processing in the cell.

In some embodiments, the Frizzled has an amino acid sequence of:

(SEQ ID NO: 31)
MRPRSALPRLLLPLLLLPAAGPAQFHGEKGISIPDHGFCQPISIPLCTD
IAYNQTIMPNLLGHTNQEDAGLEVHQFYPLVKVQCSPELRFFLCSMYAP
VCTVLEQAIPPCRSICERARQGCEALMNKFGFQWPERLRCEHFPRHGAE
QICVGQNHSEDGAPALLTTAPPPGLQPGAGGTPGGPGGGGAPPRYATLE
HPFHCPRVLKVPSYLSYKFLGERDCAAPCEPARPDGSMFFSQEETRFAR
LWILTWSVLCCASTFFTVTTYLVDMQRFRYPERPIIFLSGCYTMVSVAY
IAGFVLQERVVCNERFSEDGYRTVVQGTKKEGCTILFMMLYFFSMASSI
WWVILSLTWFLAAGMKWGHEAIEANSQYFHLAAWAVPAVKTITILAMGQ
IDGDLLSGVCFVGLNSLDPLRGFVLAPLFVYLFIGTSFLLAGFVSLFRI
RTIMKHDGTKTEKLERLMVRIGVFSVLYTVPATIVIACYFYEQAFREHW
ERSWVSQHCKSLAIPCPAHYTPRMSPDFTVYMIKYLMTLIVGITSGFWI
WSGKTLHSWRKFYTRLTNSRHGETTV.

GenBank™ accession number L37882 provides another exemplary human Frizzled nucleic acid sequence.

The Vangl family proteins are components of the non-canonical Wnt Planar cell polarity pathway. In humans, at least three Vangl proteins, Vangl1 and Vangl2, belong to this family. The term “Van Gogh-like Protein,” “Vang-like Protein,” “VANGL planar cell polarity protein,” or “Vangl” encompasses a polypeptide (“polypeptide” and “protein” are used interchangeably herein), including any native polypeptide, from any vertebrate source, including mammals such as primates (e.g., humans and cynomolgus monkeys (cynomolgus)), dogs, and rodents (e.g., mice and rats), unless otherwise indicated. In certain embodiments, the terms include “related Vangl polypeptides,” including SNP variants thereof. The term “Vangl” also encompasses “full-length,” unprocessed Vangl as well as any form of Vangl that results from processing in the cell.

In some embodiments the Vangl1 has an amino acid sequence of:

(SEQ ID NO: 32)
MDTESTYSGYSYYSSHSKKSHRQGERTRERHKSPRNKDGRGSEKSVTIQ
PPTGEPLLGNDSTRTEEVQDDNWGETTTAITGTSEHSISQEDIARISKD
MEDSVGLDCKRYLGLTVASFLGLLVFLTPIAFILLPPILWRDELEPCGT
ICEGLFISMAFKLLILLIGTWALFFRKRRADMPRVFVFRALLLVLIFLF
VVSYWLFYGVRILDSRDRNYQGIVQYAVSLVDALLFIHYLAIVLLELRQ
LQPMFTLQVVRSTDGESRFYSLGHLSIQRAALVVLENYYKDFTIYNPNL
LTASKFRAAKHMAGLKVYNVDGPSNNATGQSRAMIAAAARRRDSSHNEL
YYEEAEHERRVKKRKARLVVAVEEAFIHIQRLQAEEQQKAPGEVMDPRE
AAQAIFPSMARALQKYLRITRQQNYHSMESILQHLAFCITNGMTPKAFL
ERYLSAGPTLQYDKDRWLSTQWRLVSDEAVTNGLRDGIVFVLKCLDFSL
VVNVKKIPFIILSEEFIDPKSHKFVLRLQSETSV.

GenBank™ accession number NM_138959 provides another exemplary human Vangl1 nucleic acid sequence.

In some embodiments, the Vangl2 has an amino acid sequence of:

(SEQ ID NO: 33)
MDTESQYSGYSYKSGHSRSSRKHRDRRDRHRSKSRDGGRGDKSVTIQAP
GEPLLDNESTRGDERDDNWGETTTVVTGTSEHSISHDDLTRIAKDMEDS
VPLDCSRHLGVAAGATLALLSFLTPLAFLLLPPLLWREELEPCGTACEG
LFISVAFKLLILLLGSWALFFRRPKASLPRVFVLRALLMVLVFLLVVSY
WLFYGVRILDARERSYQGVVQFAVSLVDALLFVHYLAVVLLELRQLQPQ
FTLKVVRSTDGASRFYNVGHLSIQRVAVWILEKYYHDFPVYNPALLNLP
KSVLAKKVSGFKVYSLGEENSTNNSTGQSRAVIAAAARRRDNSHNEYYY
EEAEHERRVRKRRARLVVAVEEAFTHIKRLQEEEQKNPREVMDPREAAQ
AIFASMARAMQKYLRTTKQQPYHTMESILQHLEFCITHDMTPKAFLERY
LAAGPTIQYHKERWLAKQWTLVSEEPVTNGLKDGIVFLLKRQDFSLVVS
TKKVPFFKLSEEFVDPKSHKFVMRLQSETSV.

GenBank™ accession number NM_020335 provides another exemplary human Vangl2 nucleic acid sequence.

The term “receptor-like tyrosine kinase” or “Ryk” encompasses a polypeptide (“polypeptide” and “protein” are used interchangeably herein), including any native polypeptide, from any vertebrate source, including mammals such as primates (e.g., humans and cynomolgus monkeys (cynomolgus)), dogs, and rodents (e.g., mice and rats), unless otherwise indicated. In certain embodiments, the terms include “related Ryk polypeptides,” including SNP variants thereof. The term “Ryk” also encompasses “full-length,” unprocessed Vangl as well as any form of Vangl that results from processing in the cell.

In some embodiments, the Ryk has an amino acid sequence of:

(SEQ ID NO: 34)
MRGAARLGRPGRSCLPGARGLRAPPPPPLLLLLALLPLLPAPGAAAAPA
PRPPELQSASAGPSVSLYLSEDEVRRLIGLDAELYYVRNDLISHYALSF
SSLLVPETNFLHFTWHAKSKVEYKLGFQVDNVLAMDMPQVNISVQGEVP
RTLSVFRVELSCTGKVDSEVMILMQLNLTVNSSKNFTVLNFKRRKMCYK
KLEEVKTSALDKNTSRTIYDPVHAAPTTSTRVFYISVGVCCAVIFLVAI
ILAVLHLHSMKRIELDDSISASSSSQGLSQPSTQTTQYLRADTPNNATP
ITSSLGYPTLRIEKNDLRSVTLLEAKGKVKDIAISRERITLKDVLQEGT
FGRIFHGILIDEKDPNKEKQAFVKTVKDQASEIQVTMMLTESCKLRGLH
HRNLLPITHVCIEEGEKPMVILPYMNWGNLKLFLRQCKLVEANNPQAIS
QQDLVHMAIQIACGMSYLARREVIHKDLAARNCVIDDTLQVKITDNALS
RDLFPMDYHCLGDNENRPVRWMALESLVNVMACCWALDPEERPKFQQLV
QCLTEFHAALGAYV.

GenBank™ accession number NM_001005861 provides another exemplary human Ryk nucleic acid sequence.

The term “Wnt” encompasses a polypeptide (“polypeptide” and “protein” are used interchangeably herein), including any native polypeptide, from any vertebrate source, including mammals such as primates (e.g., humans and cynomolgus monkeys (cynomolgus)), dogs, and rodents (e.g., mice and rats), unless otherwise indicated. In certain embodiments, the terms include “related Wnt polypeptides,” including SNP variants thereof. The term “Wnt” also encompasses “full-length,” unprocessed Wnt as well as any form of Wnt that results from processing in the cell. Thus, for example, in some embodiments, Wnt can refer to the full-length amino acid sequence encoded by any of the Wnt-encoding genes identified in human (J. R. Miller, Genome Biol. 2002; 3(1):REVIEWS3001. Epub 2001 Dec. 28). In some embodiments, Wnt can refer to a murine Wnt protein, such as murine Wnt4 (Miller 2002; Supra). In some embodiments, Wnt can also refer to a polypeptide that contains the full-length consecutive sequence of a Wnt, and at least one additional amino acid residues. In some embodiments, Wnt can refer to a polypeptide that is or contains a truncated sequence of a Wnt protein, a mutated Wnt protein, as long as the amino acid sequence retains an acceptable level of the equivalent biological activity of a full-length Wnt protein.

The term “neuron” encompasses a neuron and a portion or portions thereof (e.g., the neuron cell body, an axon, or a dendrite). The term “neuron” as used herein denotes nervous system cells that include a central cell body or soma, and two types of extensions or projections: dendrites, by which, in general, the majority of neuronal signals are conveyed to the cell body, and axons, by which, in general, the majority of neuronal signals are conveyed from the cell body to effector cells, such as target neurons or muscle. Neurons can convey information from tissues and organs into the central nervous system (afferent or sensory neurons) and transmit signals from the central nervous systems to effector cells (efferent or motor neurons). Other neurons, designated interneurons, connect neurons within the central nervous system (the brain and spinal column). Certain specific examples of neuron types that may be subject to treatment or methods according to the invention include cerebellar granule neurons, dorsal root ganglion neurons, and cortical neurons.

“Synapse” or is a term of art and refers to the communicating cell-cell junctions that allow signals to pass from a nerve cell (i.e. neuron) to a target cell (e.g. a neighboring neuron or a muscle cell). A synapse is composed of a presynaptic membrane of a presynaptic cell (e.g., a membrane of an axon of a neuron) and a postsynaptic membrane of a postsynaptic cell (e.g., a membrane of dendrite of a neuron, or of a specialized region of a muscle or a secretory cell), with the presynaptic and postsynaptic membranes typically opposing each other. The gap between the opposing synaptic membranes of a synapse is known as the synaptic cleft. A neuron typically forms a plurality of synapse with its neighboring cells. The neuron typically serves as the presynaptic cell for synapses formed on its axon, and as the postsynaptic cell for synapses formed on its dendrite. Accordingly, a “presynaptic site of a neuron” as used herein refers to the synaptic site of a neuron, for which synapses the neuron serves as the presynaptic cell; a “postsynaptic site of a neuron” as used herein refers to the synaptic site of a neuron, for which synapse the neuron serves as the postsynaptic cell.

The term “neuronal degeneration” is used broadly and refers to any pathological changes in neuronal cells, including, without limitation, death or loss of neuronal cells, any changes that precede cell death, and any reduction or loss of an activity or a function of the neuronal cells. One underlying reason for the reduction or loss of an activity or a function of neurons is the reduction in the number of functional synapses formed by the neurons. The pathological changes may be spontaneous or may be induced by any event and include, for example, pathological changes associated with apoptosis. The neurons may be any neurons, including without limitation sensory, sympathetic, parasympathetic, or enteric, e.g., dorsal root ganglia neurons, motor neurons, and central neurons, e.g., neurons from the brain. Neuronal degeneration or cell loss is a characteristic of a variety of neurological diseases or disorders, e.g., neurodegenerative diseases or disorders. In some embodiments, the neuron is a sensory neuron. In some embodiments, the neuron is a motor neuron. In some embodiments, the neuron is a neuron in the brain.

As used herein, and unless otherwise specified, the term “subject” refers to an animal that is the object of treatment, observation and/or experiment. “Animal” includes vertebrates and invertebrates, such as fish, shellfish, reptiles, birds, and, in particular, mammals. “Mammal” includes, but not limited to, mice, rats, rabbits, guinea pigs, dogs, cats, sheep, goats, cows, horses, primates, such as monkeys, chimpanzees, apes, and humans.

The term “effective amount” as used herein refers to the amount of an antibody or pharmaceutical composition provided herein which is sufficient to result in the desired outcome.

The term “therapeutically effective amount” as used herein refers to the amount of an agent (e.g., an antibody provided herein or any other agent described herein) that is sufficient to reduce and/or ameliorate the severity and/or duration of a given disease, disorder, or condition, and/or a symptom related thereto (e.g., Alzheimer's disease). A “therapeutically effective amount” of a substance/molecule/agent of the present disclosure may vary according to factors such as the disease state, age, sex, and weight of the individual, and the ability of the substance/molecule/agent to elicit a desired response in the individual. A therapeutically effective amount encompasses an amount in which any toxic or detrimental effects of the substance/molecule/agent are outweighed by the therapeutically beneficial effects. In certain embodiments, the term “therapeutically effective amount” refers to an amount of an antibody or other agent (e.g., drug) effective to “treat” a disease, disorder, or condition, in a subject or mammal.

A “prophylactically effective amount” is an amount of a pharmaceutical composition that, when administered to a subject, will have the intended prophylactic effect, e.g., preventing, delaying, or reducing the likelihood of the onset (or reoccurrence) of a disease, disorder, condition, or associated symptom(s) (e.g., Alzheimer's disease). Typically, but not necessarily, since a prophylactic dose is used in subjects prior to or at an earlier stage of a disease, disorder, or condition, a prophylactically effective amount may be less than a therapeutically effective amount. The full therapeutic or prophylactic effect does not necessarily occur by administration of one dose, and may occur only after administration of a series of doses. Thus, a therapeutically or prophylactically effective amount may be administered in one or more administrations.

The term “therapy” refers to any protocol, method, and/or agent that can be used in the prevention, management, treatment, and/or amelioration of a neuronal disorder, or condition. In certain embodiments, the terms “therapies” and “therapy” refer to a biological therapy, supportive therapy, and/or other therapies useful in the prevention, management, treatment, and/or amelioration of a neuronal disorder, disorder, or condition, known to one of skill in the art such as medical personnel.

The terms “manage,” “managing,” and “management” refer to the beneficial effects that a subject derives from a therapy (e.g., a prophylactic or therapeutic agent), which does not result in a cure of the disease. In certain embodiments, a subject is administered one or more therapies (e.g., prophylactic or therapeutic agents to “manage” a neuronal disorder, one or more symptoms thereof, so as to prevent the progression or worsening of the disease.

The terms “prevent,” “preventing,” and “prevention” refer to reducing the likelihood of the onset (or recurrence) of a disease, disorder, condition, or associated symptom(s) (e.g., Alzheimer's disease).

“Administer” or “administration” refers to the act of injecting or otherwise physically delivering a substance as it exists outside the body into a patient, such as by mucosal, intradermal, intravenous, intramuscular delivery, and/or any other method of physical delivery described herein or known in the art. When a disease, disorder, condition, or a symptom thereof, is being treated, administration of the substance typically occurs after the onset of the disease, disorder, condition, or symptoms thereof. When a disease, disorder, condition, or symptoms thereof, are being prevented, administration of the substance typically occurs before the onset of the disease, disorder, condition, or symptoms thereof.

The term “inhibition” or “inhibit,” when used herein, refers to partial (such as, 1%, 2%, 5%, 10%, 20%, 25%, 50%, 75%, 90%, 95%, 99%) or complete (i.e., 100%) inhibition.

The term “attenuate,” “attenuation,” or “attenuated,” when used herein, refers to partial (such as, 1%, 2%, 5%, 10%, 20%, 25%, 50%, 75%, 90%, 95%, 99%) or complete (i.e., 100%) reduction in a property, activity, effect, or value.

The terms “increase,” “enhance,” or “promote,” when used herein, refers to an increase (such as, 10%, 20%, 50%, 100%, 200%, 500%, or to a greater extent) in a property, activity, effect or value.

“Substantially all” refers to at least about 60%, at least about 65%, at least about 70%, at least about 75%, at least about 80%, at least about 85%, at least about 90%, at least about 95%, at least about 98%, at least about 99%, or about 100%.

The phrase “substantially similar” or “substantially the same” denotes a sufficiently high degree of similarity between two numeric values (e.g., one associated with an antibody of the present disclosure and the other associated with a reference antibody) such that one of skill in the art would consider the difference between the two values to be of little or no biological and/or statistical significance within the context of the biological characteristic measured by the values (e.g., K_{D} values). For example, the difference between the two values may be less than about 50%, less than about 40%, less than about 30%, less than about 20%, less than about 10%, or less than about 5%, as a function of the value for the reference antibody.

The phrase “substantially increased,” “substantially reduced,” or “substantially different,” as used herein, denotes a sufficiently high degree of difference between two numeric values (e.g., one associated with an antibody of the present disclosure and the other associated with a reference antibody) such that one of skill in the art would consider the difference between the two values to be of statistical significance within the context of the biological characteristic measured by the values. For example, the difference between said two values can be greater than about 10%, greater than about 20%, greater than about 30%, greater than about 40%, or greater than about 50%, as a function of the value for the reference antibody.

Methods of Modulation

Loss of glutamatergic synapses is an important early step in pathogenesis of Alzheimer's disease and is thought to be induced by oligomeric amyloid β (Aβ). For example, synapse loss correlates with cognitive decline in the Alzheimer's disease and precedes neurofibrillary tangle formation and neuronal death. Overproduction of amyloid β (Aβ) has been associated with the Alzheimer's disease. Aβ readily self-associates to form a range of neurotoxic soluble oligomers, and insoluble deposited fibers. Soluble Aβ oligomers induce loss of synapse, loss of long-term potentiation (LTP), increase of long-term depression (LTD), and decrease of dendritic spine density.

Synapse formation involves recognition of specific postsynaptic targets by growing axons, formation of initial contacts, and subsequent elaboration of the transmitter release machinery and the postsynaptic apparatus at contact sites. Synapse maintenance involves stabilization of formed contacts between pre- and postsynaptic elements. Without being bound by the theory, it is contemplated in the present disclosure that the planar cell polarity (PCP) pathway plays an important role for glutamatergic synapse formation and maintenance in both developing and adult individuals. Particularly, PCP pathway components Frizzled, Disheveled, Vangl, and Celsr are found to be located in excitatory synapses in the adult, and their level changes in aging brains and the brains of Alzheimer's disease patients. Celsr, a PCP pathway component, mediates formation of excitatory synapses. Particularly, Celsr molecules expressed by presynaptic and postsynaptic cells form intercellular complexes across the synaptic cleft. Celsr also forms intracellular complexes with Frizzled to stabilize synaptic assembly. Another PCP pathway component, Vangl, dissembles glutamatergic synapses by disrupting the intracellular complex formed by Celsr and Frizzled.

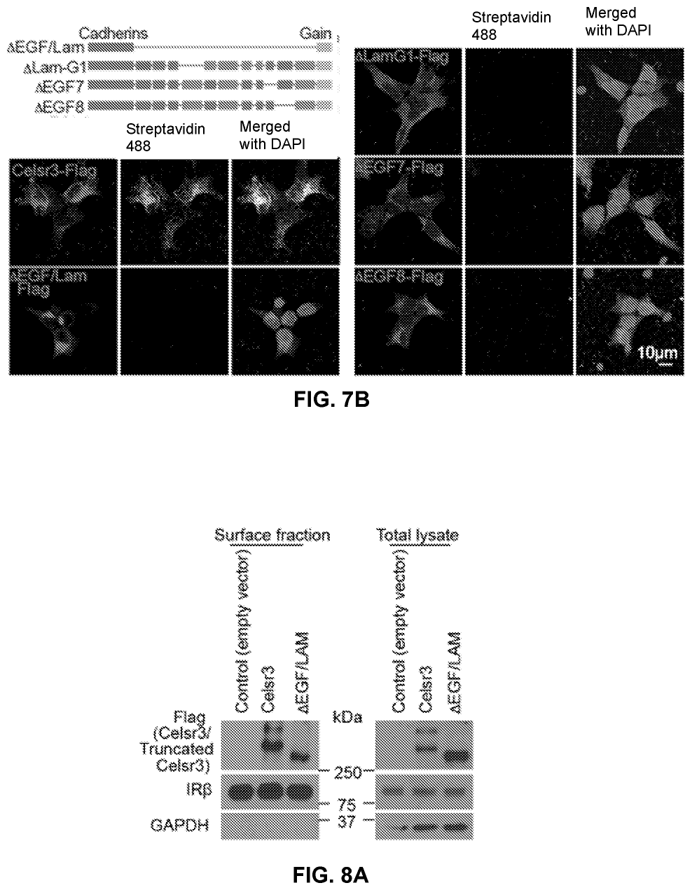

The cadherin EGF LAG seven-pass G-type receptors (CELSRs) are a special subgroup of adhesion G protein-coupled receptors (GPCRs), which are regulators of many biological processes such as neuronal/endocrine cell differentiation, vessel valve formation and the control of planar cell polarity during embryonic development. All three members of the Celsr family (Celsr1-3) have large ecto-domains that form homophilic interactions and encompass more than 2,000 amino acids. The Celsr genes have been cloned and the domain structures of the Celsr proteins are known (Wang et al. J Neurochem, 2014 December; 131(6): 699-711). For example, FIG. 7A shows the extracellular domain structure of mouse Celsr3 protein, including 9 cadherin domains, 8 EGF domains and 3 laminin domains.

Without being bound by the theory, it is contemplated in the present disclosure that Aβ mediates synaptotoxicity, including inducing loss of excitatory synapses, by targeting the PCP pathway. Particularly, it is contemplated in this disclosure that Aβ binds to Celsr and weakens the protein complex formed by PCP components thereby promotes disassembly of neuronal synapses by Vangl. Particularly, Aβ binds to one or more extracellular domains of Celsr. For example, the present disclosure provides data demonstrating that Aβ binds to one or more domains selected from the EGF7, EGF8, and Laminin G1 domains of Celsr3 (see Example 3). It is further contemplated that extracellular domains of Celsr 1 or Celsr 2 protein have conserved sequences corresponding to the domains of Celsr3 proteins responsible for binding with Aβ also retain similar functionality in terms of binding with Aβ.

Accordingly, in some embodiments, a method for reducing or preventing amyloid beta (Aβ) induced loss of synapses in a population of neurons comprises contacting the neurons with an effective amount of (a) a Celsr agonist; (b) Frizzled agonist; (c) Vangl inhibitor; (d) Ryk inhibitor; (e) Aβ inhibitor; or (f) any combination of (a) to (e). In some embodiments, the population of neurons are in a subject, and wherein the contacting step comprises administering the (a) a Celsr agonist; (b) Frizzled agonist; (c) Vangl inhibitor; (d) Ryk inhibitor; (e) Aβ inhibitor; or (f) any combination of (a) to (e) to the subject. In some embodiments, the subject has, or is at risk of developing, a neurodegenerative disease that is resulted from loss of synapse in the nervous system. In some embodiments, the neurodegenerative disease includes Alzheimer's Disease and Parkinson's disease.

In some embodiments, a method for reducing or preventing amyloid beta (Aβ) induced loss of synapses in a population of neurons comprises contacting the neurons with an effective amount of a Celsr agonist. In some embodiments, the Celsr agonist exerts the agonistic activity by increasing the amount of Celsr protein produced by a cell (e.g., a neuron).

In some embodiments, the Celsr agonist exerts the agonistic activity by reducing endocytosis of Celsr located in the synaptic site of a neuron. In some embodiments, the Celsr agonist exerts the agonistic activity by reducing endocytosis of Celsr located in the presynaptic membrane of a neuron. In some embodiments, the Celsr agonist exerts the agonistic activity by reducing endocytosis of Celsr located in the postsynaptic membrane of a neuron. In some embodiments, the Celsr agonist exerts the agonistic activity by reducing endocytosis of Celsr located in both the presynaptic membrane and postsynaptic membranes of a population of neurons. In some embodiments, the synapses are excitatory synapses. In specific embodiments, the synapses are glutamatergic synapses.

In some embodiments, the Celsr agonist exerts the agonistic activity by increasing transportation of Celsr to the synaptic site of a neuron. In some embodiments, the Celsr agonist exerts the agonistic activity by increasing assembly of Celsr into the presynaptic membrane of a neuron. In some embodiments, the Celsr agonist exerts the agonistic activity by increasing assembly of Celsr into the postsynaptic membrane of a neuron. In some embodiments, the Celsr agonist exerts the agonistic activity by increasing assembly of Celsr into both the presynaptic membrane and postsynaptic membranes of a population of neurons. In particular embodiments, the synapses are excitatory synapses. In particular embodiments, the synapses are glutamatergic synapses.

In some embodiments, a method for reducing or preventing amyloid beta (Aβ) induced loss of synapses in a population of neurons comprises contacting the neurons with an effective amount of a Frizzled agonist. In some embodiments, the Frizzled agonist exerts the agonistic activity by increasing the amount of Frizzled protein produced by a cell (e.g., a neuron).

In some embodiments, the Frizzled agonist exerts the agonistic activity by reducing endocytosis of Frizzled located in the synaptic site of a neuron. In some embodiments, the Frizzled agonist exerts the agonistic activity by reducing endocytosis of Frizzled located in the presynaptic membrane of a neuron. In some embodiments, the Frizzled agonist exerts the agonistic activity by reducing endocytosis of Frizzled located in the postsynaptic membrane of a neuron. In some embodiments, the Frizzled agonist exerts the agonistic activity by reducing endocytosis of Frizzled located in both the presynaptic membrane and postsynaptic membranes of a population of neurons. In some embodiments, the synapses are excitatory synapses. In specific embodiments, the synapses are glutamatergic synapses.

In some embodiments, the Frizzled agonist exerts the agonistic activity by increasing transportation of Frizzled to the synaptic site of a neuron. In some embodiments, the Frizzled agonist exerts the agonistic activity by increasing assembly of Frizzled into the presynaptic membrane of a neuron. In some embodiments, the Frizzled agonist exerts the agonistic activity by increasing assembly of Frizzled into the postsynaptic membrane of a neuron. In some embodiments, the Frizzled agonist exerts the agonistic activity by increasing assembly of Frizzled into both the presynaptic membrane and postsynaptic membranes of a population of neurons. In particular embodiments, the synapses are excitatory synapses. In particular embodiments, the synapses are glutamatergic synapses.

In some embodiments, a method for reducing or inhibiting Aβ induced loss of synapses in a population of neurons comprises contacting the neurons with an effective amount of an Aβ inhibitor that blocks binding of Aβ to Celsr. In some embodiments, Celsr is located at the presynaptic site of a neuron. In some embodiments, Celsr is located on the postsynaptic membrane of a synapse. In some embodiments, Celsr is located at the postsynaptic site of a neuron. In some embodiments, Celsr is located at the postsynaptic membrane of a synapse. In some embodiments, Celsr is located on both the presynaptic and postsynaptic site of a population of neurons. In some embodiments, Celsr is located on both the presynaptic and postsynaptic membranes of a synapse. In particular embodiments, the synapses are excitatory synapses. In particular embodiments, the synapses are glutamatergic synapses.

According to the present disclosure, various known isoforms of Celsr can be the target of Aβ to induce synapse loss. Accordingly, in some embodiments, the Aβ inhibitor specifically binds to one of the Celsr isoforms, thereby blocking binding of Aβ to such Celsr isoform. For example, in some embodiments, the Aβ inhibitor specifically binds to Celsr isoform Celsr 1. In some embodiments, the Aβ inhibitor specifically binds to Celsr isoform Celsr 1. In some embodiments, the Aβ inhibitor specifically binds to Celsr isoform Celsr 3.

In alternative embodiments, the Aβ inhibitor is capable of specifically binding to multiple Celsr isoforms, thereby blocking binding of Aβ to such Celsr isoforms. For example, in some embodiments, the Aβ inhibitor specifically binds to Celsr isoforms Celsr 1 and Celsr2. In some embodiments, the Aβ inhibitor specifically binds to Celsr isoforms Celsr 1 and Celsr 3. In some embodiments, the Aβ inhibitor specifically binds to Celsr isoforms Celsr 2 and Celsr 3.

In particular embodiments, the Aβ inhibitor is capable of specifically binding to multiple Celsr isoforms, and exhibits preferential binding to one isoform over another isoform. In specific embodiments, the Aβ inhibitor preferentially binds to Celsr 3 over Celsr 2. In specific embodiments, the Aβ inhibitor binds to Celsr 3 with a K_{D} less than the K_{D} exhibited for binding with Celsr 2. In specific embodiments, the Aβ inhibitor binds to Celsr 3 with a K_{D} of less than about 95%, less than about 90%, less than about 80%, less than about 70%, less than about 60%, less than about 50%, less than about 40%, less than about 30%, less than about 20%, less than about 10%, or less than about 5% of the K_{D} exhibited for binding with Celsr 2.

In some embodiments, the Aβ inhibitor competes with Aβ for binding to the EGF7, EGF8, and/or Laminin G1 domains of Celsr. In particular embodiments, the Aβ inhibitor competes with Aβ for binding to the EGF7 domain of Celsr. In particular embodiments, the Aβ inhibitor competes with Aβ for binding to the EGF8 domain of Celsr. In particular embodiments, the Aβ inhibitor competes with Aβ for binding to the Laminin G1 domain of Celsr. In particular embodiments, the Aβ inhibitor competes with Aβ for binding to both the EGF7 and EGF8 domains of Celsr. In particular embodiments, the Aβ inhibitor competes with Aβ for binding to both the EGF7 domain and the Laminin G1 domain of Celsr. In particular embodiments, the Aβ inhibitor competes with Aβ for binding to both the EGF8 domain and the Laminin G1 domain of Celsr. In particular embodiments, the Aβ inhibitor competes with Aβ for binding to the EGF7 domain, EGF8 domain and the Laminin G1 domain of Celsr.

In some embodiments, the Aβ is an Aβ peptide in monomeric form. In other embodiments, the Aβ is multiple Aβ peptides aggregated in the oligomeric form. Particularly, according to the present disclosure, different species of Aβ monomers (e.g., having different lengths and/or sequences) can aggregate in the oligomeric form. For example, different species of Aβ monomers can be peptides ranging in size from 37 to 49 amino acid residues, which are produced through the proteolytic processing of the amyloid precursor protein (APP) by β-secretase and γ-secretase. Alternatively, an Aβ oligomer can also contain several Aβ monomers of the same species, such as but not limited to Aβ42 and other species of Aβ monomers known in the art.

In specific embodiments, an Aβ oligomer comprises at least 2 Aβ monomers, such as at least 2, 3, 4, 5, 6, 7, 8, 9, 10, 11, 12, 13, 14, 15, 16, 17, 18, 19 or 20 Aβ monomers. In specific embodiments, an Aβ oligomer comprises about 2 to 8 Aβ monomers. In specific embodiments, an Aβ oligomer comprises about 2 to 6 Aβ monomers. In specific embodiments, an Aβ oligomer comprises about 2 to 4 Aβ monomers. In specific embodiments, an Aβ oligomer comprises 2 Aβ monomers. In specific embodiments, an Aβ oligomer comprises 3 Aβ monomers. In specific embodiments, an Aβ oligomer comprises about 4 Aβ monomers. In any of the embodiments described in this paragraph, the Aβ monomers found in an Aβ oligomer can be of the same or different species. In any of the embodiments described in this paragraph, the Aβ monomer found in an Aβ oligomer is Aβ42.

In some embodiments, the Aβ inhibitor is an anti-Celsr antibody or antigen binding fragment thereof. In some embodiments, the anti-Celsr antibody specifically binds to an epitope in the EGF7 domain of Celsr3. In some embodiments, the anti-Celsr antibody specifically binds to an epitope in the EGF8 domain of Celsr3. In some embodiments, the anti-Celsr antibody specifically binds to an epitope in the Laminin G1 domain of Celsr3.

In some embodiments, the Aβ inhibitor is an anti-Celsr antibody or antigen binding fragment thereof that specifically binds to the Celsr isoform Celsr 1. In some embodiments, the Aβ inhibitor is an anti-Celsr antibody or antigen binding fragment thereof that specifically binds to the Celsr isoform Celsr 2. In some embodiments, the Aβ inhibitor is an anti-Celsr antibody or antigen binding fragment thereof that specifically binds to the Celsr isoform Celsr3.

In alternative embodiments, the Aβ inhibitor is an anti-Celsr antibody or antigen binding fragment thereof that specifically binds to multiple Celsr isoforms selected from Celsr 1, Celsr 2 and Celsr 3. In specific embodiments, the Aβ inhibitor is an anti-Celsr antibody or antigen binding fragment thereof that preferentially binds to Celsr 3 over Celsr 2. In specific embodiments, the anti-Celsr antibody or antigen binding fragment thereof binds to Celsr 3 with a K_{D} less than the K_{D} exhibited for binding with Celsr 2. In specific embodiments, the anti-Celsr antibody or antigen binding fragment thereof binds to Celsr 3 with a K_{D} of less than about 95%, less than about 90%, less than about 80%, less than about 70%, less than about 60%, less than about 50%, less than about 40%, less than about 30%, less than about 20%, less than about 10%, or less than about 5% of the K_{D} exhibited for binding with Celsr 2.

In some embodiments, the Aβ inhibitor binds to Aβ, and upon binding to Aβ, prevents or reduces Aβ binding to Celsr. In some embodiments, the Aβ inhibitor comprises the Aβ binding site of the Celsr protein and is capable of competing with Celsr for binding with Aβ. In some embodiments, the Aβ inhibitor comprises (a) one or more copy of the EGF7 domain of Celsr or functional variant thereof, (b) one or more copy of the EGF8 domain of Celsr or a functional variant thereof, (c) one or more copy of the Laminin G1 domain of Celsr or a functional variant thereof, or (d) any combination of (a) to (c). In any embodiments described in this paragraph, the functional variant of a Ceslr3 domain can have at least about 50%, 60%, 70%, 80%, 90%, 95% or 98% sequence homology to the native Celsr3 domain sequence.

In specific embodiments, the Aβ inhibitor comprises a Laminin G1 domain of Celsr that is of human origin and has the amino acid sequence of:

(SEQ ID NO: 35)
VAARSFPPSSFVMFRGLRQRFHLTLSLSFATVQQSGLLFYNGRLNEKHD
FLALELVAGQVRLTYSTGESNTVVSPTVPGGLSDGQWHTVHLRYYNKPR
TDALGGAQGPSKDKVAVLSVDDCDVAVALQFGAEIGNYSCAAAGVQTSS
KKSLDLTGPLLLGGVPNLPENFPVSHKDFIGCMRDLHIDGRRVDMAAFV
ANNGTMAGC,

or a functional variant having at least about 50%, 60%, 70%, 80%, 90%, 95% or 98% sequence homology to SEQ ID NO: 35. In specific embodiments, the functional variant of the Laminin G1 domain is capable of binding to Aβ and blocks the binding of Aβ to Celsr at the synapses. In specific embodiments, the Aβ inhibitor is a fusion protein comprising more than 1, 2, 3, 4, or 5 copies of the Celsr Laminin G1 domain or functional variant thereof.

In specific embodiments, the Aβ inhibitor comprises a Laminin G1 domain of Celsr that is of mouse origin and has the amino acid sequence of:

(SEQ ID NO: 36)
VAARSFPPSSFVMFRGLRQRFHLTLSLSFATVQPSGLLFYNGRLNEKHD
FLALELVAGQVRLTYSTGESNTVVSPTVPGGLSDGQWHTVHLRYYNKPR
TDALGGAQGPSKDKVAVLSVDDCNVAVALQFGAEIGNYSCMAGVQTSSK
KSLDLTGPLLLGGVPNLPENFPVSHKDFIGCMRDLHIDGRRMDMAAFVA
NNGTMAGC,

or a functional variant having at least about 50%, 60%, 70%, 80%, 90%, 95% or 98% sequence homology to SEQ ID NO: 36. In specific embodiments, the functional variant of the Laminin G1 domain is capable of binding to Aβ and blocks the binding of Aβ to Celsr at the synapses. In specific embodiments, the Aβ inhibitor is a fusion protein comprising more than 1, 2, 3, 4, or 5 copies of the Celsr Laminin G1 domain or functional variant thereof.

In specific embodiments, the Aβ inhibitor comprises a EGF7 domain of Celsr that is of human origin and has the amino acid sequence of: HRMDQQCPRGWWGSPTCGPCNCDVHKGFDPNCN (SEQ ID NO: 37), or a functional variant having at least about 50%, 60%, 70%, 80%, 90%, 95% or 98% sequence homology to SEQ ID NO: 37. In specific embodiments, the functional variant of the EGF7 domain of Celsr domain is capable of binding to Aβ and blocks the binding of Aβ to Celsr at the synapses. In specific embodiments, the Aβ inhibitor is a fusion protein comprising more than 1, 2, 3, 4, or 5 copies of the Celsr EGF7 domain or functional variant thereof.

In specific embodiments, the Aβ inhibitor comprises a EGF7 domain of Celsr that is of mouse origin and has the amino acid sequence of:

(SEQ ID NO: 38)
YFGQHCEHRVDQQCPRGWWGSPTCGPCNCDVHKGFDPNCN,

or a functional variant having at least about 50%, 60%, 70%, 80%, 90%, 95% or 98% sequence homology to SEQ ID NO: 38. In specific embodiments, the functional variant of the EGF7 domain is capable of binding to Aβ and blocks the binding of Aβ to Celsr at the synapses. In specific embodiments, the Aβ inhibitor is a fusion protein comprising more than 1, 2, 3, 4, or 5 copies of the Celsr EGF7 domain or functional variant thereof.

In specific embodiments, the Aβ inhibitor comprises a EGF8 domain of Celsr that is of human or mouse origin and has the amino acid sequence of:

(SEQ ID NO: 39)
TNGQCHCKEFHYRPRGSDSCLPCDCYPVGSTSRSCA,

or a functional variant having at least about 50%, 60%, 70%, 80%, 90%, 95% or 98% sequence homology to SEQ ID NO: 39. In specific embodiments, the functional variant of the EGF8 domain is capable of binding to Aβ and blocks the binding of Aβ to Celsr at the synapses. In specific embodiments, the Aβ inhibitor is a fusion protein comprising more than 1, 2, 3, 4, or 5 copies of the Celsr EGF8 domain or functional variant thereof.

In particular embodiments, the Aβ inhibitor comprises one or more copies of the extracellular domain of Celsr, or a functional variant thereof having at least about 50%, 60%, 70%, 80%, 90%, 95% or 98% sequence homology to the native sequence of the Celsr extracellular domain. In specific embodiments, the Aβ inhibitor is a fusion protein comprising more than 1, 2, 3, 4, or 5 copies of the Celsr extracellular domain or functional variant thereof.

In some embodiments, the Aβ inhibitor comprises a fusion protein comprising a Celsr peptide fused to the Fc region of immunoglobulin. In particular embodiments, the Celsr peptide is selected from a peptide comprising the Celsr Laminin G1 domain or a functional variant thereof, a peptide comprising the Celsr Laminin EGF7 domain or a functional variant thereof, a peptide comprising the Celsr Laminin EGF8 domain or a functional variant thereof, and a peptide comprising the Celsr extracellular domain or a functional variant thereof. In various embodiments described in this paragraph, the functional variant of the Celsr peptide is capable of binding to Aβ and block the binding of Aβ to Celsr at the synapses. In various embodiments described in this paragraph, the Fc region is selected from IgG1 Fc, IgG2 Fc, IgG3 Fc, IgG4 Fc, IgA Fc, IgD Fc, IgM Fc, IgE Fe, or a functional Fc region variant thereof. In various embodiments described in this paragraph, the Fc region can be of human original.

In some embodiments, the Aβ inhibitor is an antibody or antigen binding fragment thereof that binds to Aβ, and upon binding to Aβ prevents or reduces Aβ binding to Celsr.

As described above, the present disclosure contemplates that the PCP pathway components are direct targets of oligomeric Aβ induced loss of glutamatergic synapses. Oligomeric Aβ directly bind to Celsr and assist Vangl in disassembling synapses. For example, Example 1 shows that Vangl is involved in Aβ induced synapse loss; Example 2 shows that Vangl disrupts intercellular complexes formed by PCP pathway components; and Example 4 shows Vangl conditional knock-out (cKO) reduces glutamatergic synapse loss in a mouse model of Alzheimer's disease. Accordingly, in some embodiments, the present method for reducing or preventing Aβ induced loss of synapses in a population of neurons, comprises contacting the neurons with an effective amount of Vangl inhibitor. In some embodiments, the synapses are excitatory synapses. In specific embodiments, the synapses are glutamatergic synapses.

In some embodiments, the Vangl inhibitor exerts the inhibitory function by reducing the amount of Vangl protein produced by a cell. In some embodiments, the Vangl inhibitor comprises a nucleic acid molecule. In some embodiments, the nucleic acid molecule is a microRNA, siRNA or CRISPR-based gene editing construct that reduce or inhibits expression of Vangl encoding gene.

In some embodiments, the Vangl inhibitor exerts the inhibitory function by preventing Vangl from binding to one or more PCP pathway components present in the synaptic site of a neuron. In particular embodiments, the Vangl inhibitor prevents Vangl from binding to an intracellular complex comprising Celsr and Frizzled in the presynaptic site of a neuron. In particular embodiments, the Vangl inhibitor prevents Vangl from binding to an intracellular complex comprising Celsr and Frizzled located in the presynaptic membrane of a synapse. In some embodiments, the Vangl inhibitor prevents Vangl from binding to an intercellular complex comprising Celsr proteins located in the presynaptic and postsynaptic sites of neurons. In some embodiments, the Vangl inhibitor prevents Vangl from binding to an intercellular complex comprising Celsr proteins located in the presynaptic and postsynaptic membranes of a synapse. In some embodiments, the synapses are excitatory synapses. In specific embodiments, the synapses are glutamatergic synapses.

In some embodiments, the Vangl inhibitor prevents Vangl from binding to an intercellular complex comprising Celsr proteins located in the presynaptic and postsynaptic sites of a neuron and Frizzled located in the presynaptic site of a neuron. In some embodiments, the Vangl inhibitor prevents Vangl from binding to an intercellular complex comprising Celsr proteins located in the presynaptic and postsynaptic membranes of a synapse and Frizzled located in the presynaptic membrane of the synapse. In some embodiments, the synapses are excitatory synapses. In specific embodiments, the synapses are glutamatergic synapses.

In particular embodiments, the Vangl inhibitor prevents Vangl from binding to Frizzled in the presynaptic site of a neuron. In some embodiments, the synapses are excitatory synapses. In specific embodiments, the synapses are glutamatergic synapses. In particular embodiments, the Vangl inhibitor prevents Vangl from binding to Frizzled in the presynaptic membrane of a neuron. In some embodiments, the synapses are excitatory synapses. In specific embodiments, the synapses are glutamatergic synapses.

In particular embodiments, the Vangl inhibitor prevents Vangl located in the postsynaptic site from binding to an intracellular complex comprising Celsr and Frizzled in the presynaptic site of a neuron. In particular embodiments, the Vangl inhibitor prevents Vangl located in the postsynaptic membrane of a synapse from binding to an intracellular complex comprising Celsr and Frizzled in the presynaptic membrane of the synapse. In some embodiments, the synapses are excitatory synapses. In specific embodiments, the synapses are glutamatergic synapses.

In some embodiments, the Vangl inhibitor prevents Vangl located in the postsynaptic site from binding to an intercellular complex comprising Celsr proteins located in the presynaptic and postsynaptic sites of neurons. In some embodiments, the Vangl inhibitor prevents Vangl located in the postsynaptic membrane of a synapse from binding to an intercellular complex comprising Celsr proteins located in the presynaptic and postsynaptic membranes of the synapse. In some embodiments, the synapses are excitatory synapses. In specific embodiments, the synapses are glutamatergic synapses.

In some embodiments, the Vangl inhibitor prevents Vangl located in the postsynaptic site from binding to an intercellular complex comprising Celsr proteins located in the presynaptic and postsynaptic sites of a neuron and Frizzled located in the presynaptic site of a neuron. In some embodiments, the Vangl inhibitor prevents Vangl located in the postsynaptic membrane of a synapse from binding to an intercellular complex comprising Celsr proteins located in the presynaptic and postsynaptic membranes of the synapse and Frizzled located in the presynaptic membrane of the synapse. In some embodiments, the synapses are excitatory synapses. In specific embodiments, the synapses are glutamatergic synapses.

In particular embodiments, the Vangl inhibitor prevents Vangl located in the postsynaptic site from binding to Frizzled in the presynaptic site of a neuron. In particular embodiments, the Vangl inhibitor prevents Vangl located in the postsynaptic membrane of a synapse from binding to Frizzled in the presynaptic membrane of the synapse. In some embodiments, the synapses are excitatory synapses. In specific embodiments, the synapses are glutamatergic synapses.

In some embodiments, the Vangl inhibitor exerts the inhibitory function by preventing Vangl from disrupting intercellular complex formed at synapses by one or more PCP pathway components. In particular embodiments, the Vangl inhibitor prevents Vangl from disrupting an intercellular complex comprising Celsr proteins located in the presynaptic and postsynaptic sites of neurons. In some embodiments, the Vangl inhibitor prevents Vangl from disrupting an intercellular complex comprising Celsr proteins located in the presynaptic and postsynaptic membranes of a synapse. In some embodiments, the synapses are excitatory synapses. In specific embodiments, the synapses are glutamatergic synapses.

In some embodiments, the Vangl inhibitor exerts the inhibitory function by preventing Vangl from disrupting intracellular complex formed by one or more PCP pathway components. In particular embodiments, the Vangl inhibitor prevents Vangl from disrupting intracellular complex comprising Celsr and Frizzled in the presynaptic site of a neuron. In particular embodiments, the Vangl inhibitor prevents Vangl from disrupting an intracellular complex comprising Celsr and Frizzled located in the presynaptic membrane of a synapse. In some embodiments, the synapses are excitatory synapses. In specific embodiments, the synapses are glutamatergic synapses.

In some embodiments, the Vangl inhibitor is an antagonistic antibody specifically binding to Vangl, or a molecule comprising the antigen binding fragment of the anti-Vangl antibody. In some embodiments, the Vangl inhibitor is a small molecule compound.

Without being bound by the theory, the present disclosure also contemplates additional regulators of the PCP mediated formation of synapse. Particularly, it is further contemplated that Ryk is a receptor of Wnt, and is involved in Wnt-mediated synapse loss by regulating the PCP pathway. For example, Example 5 shows that the Wnt/Vangl2/Ryk signaling axis mediates synapse loss induced by oligomeric Aβ; Example 6 shows that Ryk is required for oligomeric amyloid beta-mediated synaptotoxicity in vivo.

Accordingly, in some embodiments, the present method for reducing or preventing Aβ induced loss of synapses in a population of neurons, comprises contacting the neurons with an effective amount of a Ryk inhibitor, either alone or in combination with one or more of an Aβ inhibitor, Vangl inhibitor, Celsr agonist and Frizzled agonist as described herein. In some embodiments, the synapses are excitatory synapses. In specific embodiments, the synapses are glutamatergic synapses.

In some embodiments, the Ryk inhibitor exerts the inhibitory function by reducing the amount of Ryk protein produced by a cell (e.g., a neuron). In some embodiments, the Ryk inhibitor comprises a nucleic acid molecule. In some embodiments, the nucleic acid molecule is a microRNA, siRNA or CRISPR-based gene editing construct that reduce or inhibits expression of Ryk encoding gene.

In some embodiments, the Ryk inhibitor exerts the inhibitory function by preventing Ryk from binding to one or more PCP pathway components present in the synaptic site of a neuron. In particular embodiments, the Ryk inhibitor prevents Ryk from binding to an intracellular complex comprising Celsr and Frizzled in the presynaptic site of a neuron. In particular embodiments, the Ryk inhibitor prevents Ryk from binding to an intracellular complex comprising Celsr and Frizzled in the presynaptic membrane of a synapse. In some embodiments, the synapses are excitatory synapses. In specific embodiments, the synapses are glutamatergic synapses.

In some embodiments, the Ryk inhibitor prevents Ryk from binding to an intercellular complex comprising Celsr proteins located in the presynaptic and postsynaptic sites of neurons. In some embodiments, the Ryk inhibitor prevents Ryk from binding to an intercellular complex comprising Celsr proteins located in the presynaptic and postsynaptic membranes of a synapse. In some embodiments, the synapses are excitatory synapses. In specific embodiments, the synapses are glutamatergic synapses.

In some embodiments, the Ryk inhibitor prevents Ryk from binding to an intercellular complex comprising Celsr proteins located in the presynaptic and postsynaptic sites of a neuron and Frizzled located in the presynaptic site of a neuron. In some embodiments, the Ryk inhibitor prevents Ryk from binding to an intercellular complex comprising Celsr proteins located in the presynaptic and postsynaptic membranes of a synapse and Frizzled located in the presynaptic membrane of the synapse. In some embodiments, the synapses are excitatory synapses. In specific embodiments, the synapses are glutamatergic synapses.

In particular embodiments, the Ryk inhibitor prevents Ryk from binding to Frizzled in the presynaptic site of a neuron. In particular embodiments, the Ryk inhibitor prevents Ryk from binding to Frizzled in the presynaptic membrane of a synapse. In some embodiments, the synapses are excitatory synapses. In specific embodiments, the synapses are glutamatergic synapses.

In some embodiments, the Ryk inhibitor prevents Ryk from binding to Wnt. In some embodiments, the synapses are excitatory synapses. In specific embodiments, the synapses are glutamatergic synapses.

In some embodiments, the Ryk inhibitor is an antagonistic antibody specifically binding to Ryk, or a molecule comprising the antigen binding fragment of the anti-Ryk antibody. In some embodiments, the Ryk inhibitor comprises one or more anti-Ryk antibodies or antigen binding fragment thereof as described in International Application No.: PCT/US2017/024494 (Published as WO 2017/172733). In some embodiments, the Ryk inhibitor is a small molecule compound.

In another aspect, provided herein are methods and related agents for modulating formation of synapses in a population of neurons. Particularly, in some embodiments, the method comprises modulating one or more planar cell polarity (PCP) signaling pathway component and/or one or more non-canonical Wnt signaling pathway component. Particularly, in some embodiments, the PCP signaling pathway component is selected from Celsr, Frizzled and Vangl. In some embodiments, the non-canonical Wnt signaling pathway component is Ryk. In particular embodiments, the method of modulating formation of synapses in a population of neurons comprises contacting the neurons with an effective amount of (a) a Celsr agonist; (b) Frizzled agonist; (c) Vangl inhibitor; (d) Ryk inhibitor; or (e) any combination of (a) to (d). In some embodiments, the population of neurons are in a subject, and the contacting step comprises administering to the subject an effective amount of the (a) a Celsr agonist; (b) Frizzled agonist; (c) Vangl inhibitor; (d) Ryk inhibitor; (e) any combination of (a) to (d). In some embodiments, the synapses are excitatory synapses. In specific embodiments, the synapses are glutamatergic synapses.

In particular embodiments, the PCP signaling pathway component is Celsr, and the method comprises contacting the neurons with an effective amount of a Celsr agonist as described herein. In particular embodiments, the Celsr is one or more isoforms selected from Celsr 1, Celsr 2 and Celsr 3. In particular embodiments, the Celsr is Celsr 3.

In particular embodiments, the PCP signaling pathway component is Frizzled, and the method comprises contacting the neurons with an effective amount of a Frizzled agonist as described herein.

In particular embodiments, the PCP signaling pathway component is Vangl, and the method comprises contacting the neurons with an effective amount of a Vangl inhibitor as described herein. In particular embodiments, the Vangl is one or more isoforms selected from Vangl 1 and Vangl 2. In particular embodiments, the Vangl is Vangl2.

In some embodiments, the non-canonical Wnt signaling pathway component is Ryk, and the method comprises contacting the neurons with an effective amount of a Ryk inhibitor as described herein.

In various embodiments described herein, the present method increases the amount or number of complexes comprising Celsr and Frizzled in a population of neurons. In some embodiments, the complexes contain one or more isomers of the Celsr protein selected from Celsr1, Celsr2 and Celsr3. In some embodiments, the complex comprises Celsr located at the presynaptic site of a neuron. In some embodiments, the complex comprises Celsr located on the presynaptic membrane of a synapse. In some embodiments, the complex comprises Celsr located at the postsynaptic site of a neuron. In some embodiments, the complex comprises Celsr located at the postsynaptic membrane of a synapse. In some embodiments, the complex comprises Celsr located on both the presynaptic and postsynaptic site of a population of neurons. In some embodiments, the complex comprises Celsr located on both the presynaptic and postsynaptic membranes of a synapse. In some embodiments, the complex comprises Frizzled located at the presynaptic site of a neuron. In some embodiments, the complex comprises Frizzled located on the presynaptic membrane of a synapse. In some embodiments, the complex further comprises Ryk. In some embodiments, the complex further comprises Wnt. In particular embodiments, the synapses are excitatory synapses. In particular embodiments, the synapses are glutamatergic synapses.

In various embodiments described herein, the present method increases the amount of complexes comprising Celsr, Frizzled and Vangl in a population of neurons. In some embodiments, the complexes contain one or more isomers of the Celsr protein selected from Celsr1, Celsr2 and Celsr3. In some embodiments, the complex comprises Celsr located at the presynaptic site of a neuron. In some embodiments, the complex comprises Celsr located on the presynaptic membrane of a synapse. In some embodiments, the complex comprises Celsr located at the postsynaptic site of a neuron. In some embodiments, the complex comprises Celsr located at the postsynaptic membrane of a synapse. In some embodiments, the complex comprises Celsr located on both the presynaptic and postsynaptic site of a population of neurons. In some embodiments, the complex comprises Celsr located on both the presynaptic and postsynaptic membranes of a synapse. In some embodiments, the complex comprises Frizzled located at the presynaptic site of a neuron. In some embodiments, the complex comprises Frizzled located on the presynaptic membrane of a synapse. In some embodiments, the complexes contain one or more isomers of the Vangl protein selected from Vangl1 and Vangl2. In some embodiments, the complex comprises Vangl located at the postsynaptic site of a neuron. In some embodiments, the complex comprises Vangl located on the postsynaptic membrane of a synapse. In some embodiments, the complex further comprises Ryk. In some embodiments, the complex further comprises Wnt. In particular embodiments, the synapses are excitatory synapses. In particular embodiments, the synapses are glutamatergic synapses.

In various embodiments described herein, the present method increases the amount of complexes comprising Celsr and Frizzled in a population of neurons. In particular embodiments, the complex comprises Celsr located in both the presynaptic and postsynaptic membranes of a synapse, and the formation of the complex is medicated by the extracellular domain of Celsr. In specific embodiments, the formation of the complexes is mediated by EGF7, EGF8, and/or Laminin G1 domains of Celsr. In specific embodiments, the formation of the complexes is mediated by Laminin G1 domains of Celsr.

In various embodiments described herein, the present method stabilizes synapses in the population of neurons. In some embodiments, the present method increases the number of synapses in the population of neurons. In some embodiments, the amount of Celsr located at presynaptic site of a neuron in the population of neurons is increased. In some embodiments, the amount of Celsr located at a presynaptic membrane of a synapse in the population of neurons is increased. In some embodiments, the amount of Celsr located at a postsynaptic site of a neuron in the population of neurons is increased. In some embodiments, the amount of Celsr located at postsynaptic membrane of a synapse in the population of neurons is increased. In some embodiments, the amount of Frizzled located at a presynaptic site of a neuron in the population of neurons is increased. In some embodiments, the amount of Frizzled located at a presynaptic membrane of a synapse in the population of neurons is increased. In particular embodiments, the synapses are excitatory synapses. In particular embodiments, the synapses are glutamatergic synapses.

In some embodiments, the population of neurons comprises a cerebellar granule neuron, a dorsal root ganglion neuron, a cortical neuron, a sympathetic neuron, or a hippocampal neuron.

Methods of Screening

In another aspect, provided herein are also methods for selecting an agent capable of modulating of synapse formation in a population of neurons. In some embodiments, the method comprises providing a population of cells comprising a first cell expressing Frizzled and Celsr and a second cell expressing Vangl; measuring a first level of association between Celsr and Frizzled; contacting a candidate agent with the population of cells; measuring a second level of association between Celsr and Frizzled; and selecting the candidate agent as the modulator if the second level of association is different from the first level of association.

In some embodiments, the population of cells are neurons, such as, but not limited to, a cerebellar granule neuron, a dorsal root ganglion neuron, a cortical neuron, a sympathetic neuron, or a hippocampal neuron. In some embodiments, the population of cells comprises exogenous nucleic acid encoding one or more proteins that are expressed on the cell surface. In particular embodiments, the cells comprise at least one exogenous nucleic acid encoding for Celsr. In specific embodiments, the cells comprise at least one exogenous nucleic acid encoding for an isomer of the Celsr protein selected from Celsr 1, Celsr 2 and Celsr 3. In particular embodiments, the cells comprise at least one exogenous nucleic acid encoding for Vangl. In specific embodiments, the cells comprise at least one exogenous nucleic acid encoding for an isomer of the Vangl protein selected from Vangl 1 and Vangl 2. In particular embodiments, the cells comprise at least one exogenous nucleic acid encoding for Frizzled.

In another aspect, provided herein are also methods for selecting an agent capable of modulating synapse formation in a population of neurons. In some embodiments, the method comprises providing a population of cells comprising a first cell expressing Frizzled and Celsr and a second cell expressing Vangl; measuring a first level of association between Celsr and Frizzled; contacting a candidate agent with the population of cells; measuring a second level of association between Celsr and Frizzled; and selecting the candidate agent as the modulator if the second level of association is different from the first level of association.

In some embodiments, the Celsr or Celsr variant is expressed on the surface of a cell. In some embodiments, the second cell further expresses Celsr. In some embodiments, the population of cells are neurons. In some embodiments, the second cell further expresses Celsr. In some embodiments, the first cell further expresses Ryk.

In some embodiments, the step of measuring comprises measuring the binding affinity between Celsr and Frizzled. In some embodiments, the step of measuring comprises measuring the binding affinity between Celsr and Vangl.

In some embodiments, the step of measuring is performed by measuring the amount of complexes comprising Celsr and Frizzled in the population of cells. In some embodiments, the amount of complexes is measured by co-immunoprecipitation of Celsr and Frizzled from the population of cells. In some embodiments, the amount of complexes is measured by co-immunoprecipitation of Celsr and Vangl from the population of cells.

In some embodiments, the step of measuring is performed by measuring the level of colocalization of Celsr and Frizzled in the cells. In some embodiments, the population of cells are neurons forming synapses, and the colocalization of Celsr and Frizzled is at synaptic sites of the neurons. In some embodiments, measuring the level of colocalization is performed by visualizing Celsr and Frizzled via microscopy.

In some embodiments, the population of cells are neurons and the step of measuring comprises measuring the amount of Celsr located at synaptic sites in the neurons. In some embodiments, the population of cells are neurons and the step of measuring comprises measuring the amount of Frizzled located at synaptic sites in the neurons. In some embodiments, the measuring comprises visualizing Celsr or Frizzled via microscopy. In some embodiments, the measuring further comprises visualizing a synaptic marker via microscopy. In some embodiments, the population of cells are neurons and the step of measuring is performed by measuring the number of synapses formed in the neurons.

In some embodiments, the candidate agent comprises a small-molecule compound, a nucleic acid, or a peptide. In some embodiments, the candidate agent comprises a microRNA, siRNA or CRISPR-based gene editing construct. In some embodiments, the candidate agent is an antibody or antigen binding fragment thereof.

In some embodiments, the method is performed in the presence of oligomeric Aβ. In some embodiments, the method is performed in the presence of Wnt.

In some embodiments, the genome of the cells comprises a heterozygous or homozygous deletion, inactivation or knock-out of the Ryk gene. In some embodiments, the genome of the cells further comprises a heterozygous or homozygous deletion, inactivation or knock-out of the Vangl gene.

In some embodiments, the population of the cells is in a non-human mammal, and the step of contacting is performed by administering the candidate agent to the non-human mammal.

In another aspect, provided herein are also methods of selecting an amyloid beta (Aβ) inhibitor that prevents or reduces Aβ-mediated neurotoxicity. In various embodiments, the method comprises contacting a candidate agent with Celsr or a Celsr variant in the presence of Aβ; and selecting the candidate agent as the Aβ inhibitor if the candidate agent reduces or inhibits binding of Aβ to the Celsr or Celsr variant.

In another aspect, provided herein are also methods for selecting an agent capable of modulating of synapse formation in a population of neurons. In some embodiments, the method comprises providing a population of cells comprising a first cell expressing Frizzled and Celsr and a second cell expressing Vangl; measuring a first level of association between Celsr and Frizzled; contacting a candidate agent with the population of cells; measuring a second level of association between Celsr and Frizzled; and selecting the candidate agent as the modulator if the second level of association is different from the first level of association.

In some embodiments, the Celsr or Celsr variant is expressed on the surface of a cell. In some embodiments, the second cell further expresses Celsr. In some embodiments, the population of cells are neurons. In some embodiments, the second cell further expresses Celsr. In some embodiments, the first cell further expresses Ryk.

In some embodiments, the measuring comprises measuring the binding affinity between Celsr and Frizzled. In some embodiments, the measuring comprises measuring the binding affinity between Celsr and Vangl.

In some embodiments, the measuring is performed by measuring the amount of complexes comprising Celsr and Frizzled in the population of cells. In some embodiments, the amount of complexes is measured by co-immunoprecipitation of Celsr and Frizzled from the population of cells. In some embodiments, the amount of complexes is measured by co-immunoprecipitation of Celsr and Vangl from the population of cells.

In some embodiments, the measuring is performed by measuring the level of colocalization of Celsr and Frizzled in the cells. In some embodiments, the population of cells are neurons forming synapses, and the colocalization of Celsr and Frizzled is at synaptic sites of the neurons. In some embodiments, measuring the level of colocalization is performed by visualizing Celsr and Frizzled via microscopy.

In some embodiments, the population of cells are neurons and the step of measuring comprises measuring the amount of Celsr located at synaptic sites in the neurons. In some embodiments, the population of cells are neurons and the step of measuring comprises measuring the amount of Frizzled located at synaptic sites in the neurons. In some embodiments, the measuring comprises visualizing Celsr or Frizzled via microscopy. In some embodiments, the measuring further comprises visualizing a synaptic marker via microscopy. In some embodiments, the population of cells are neurons and the step of measuring is performed by measuring the number of synapses formed in the neurons.

In some embodiments, the candidate agent comprises a small-molecule compound, a nucleic acid, or a peptide. In some embodiments, the candidate agent comprises a microRNA, siRNA or CRISPR-based gene editing construct. In some embodiments, the candidate agent is an antibody or antigen binding fragment thereof.

In some embodiments, the method is performed in the presence of oligomeric Aβ. In some embodiments, the method is performed in the presence of Wnt.

In some embodiments, the genome of the cells comprises a heterozygous or homozygous deletion, inactivation or knock-out of the Ryk gene. In some embodiments, the genome of the cells further comprises a heterozygous or homozygous deletion, inactivation or knock-out of the Vangl gene.

In some embodiments, the population of the cells is in a non-human mammal, and the step of contacting is performed by administering the candidate agent to the non-human mammal.

In yet another aspect, provided herein are also methods for selecting an amyloid beta (Aβ) inhibitor that prevents or reduces Aβ-mediated neurotoxicity. In specific embodiments, the method of selecting an amyloid beta (Aβ) inhibitor that prevents or reduces Aβ-mediated neurotoxicity comprises contacting a candidate agent with Celsr or a Celsr variant in the presence of Aβ; and selecting the candidate agent as the Aβ inhibitor if the candidate agent reduces or inhibits binding of Aβ to the Celsr or Celsr variant.

In some embodiments, the Celsr or Celsr variant is expressed on the surface of a cell. In some embodiments, the cell is a neuron. In some embodiments, the cell is in an in vitro cell culture. In some embodiments, the cell is a non-human mammal cell. In some embodiments, the Celsr or Celsr variant is immobilized on a solid support.

In some embodiments, the Celsr variant comprises a deletion of (a) one or more Celsr cadherin domains; (b) one or more Celsr EFG domains selected from EFG1, EFG2, EFG3, EFG4, EFG5, and EFG6; (c) one or more of Celsr laminin domains selected from Laminin-G2 and Laminin-G3; or (d) any combination of (a) to (c).

In some embodiments, the Celsr variant consists essentially of one or more extracellular domains of Celsr selected from EFG7, EFG8, and Laminin-G1. In some embodiments, the Aβ is oligomeric Aβ comprising about 2-5 Aβ monomers.

In some embodiments, the candidate agent comprises a small-molecule compound, a nucleic acid, or a peptide. In some embodiments, the candidate agent is an anti-Celsr antibody or antigen binding fragment thereof. In some embodiments, the antibody or antigen binding fragment binds to an epitope in the EFG7, EFG8, or Laminin-G1 domain of Celsr. In some embodiments, the candidate agent is an anti-Aβ antibody or antigen binding fragment thereof. In some embodiments, the candidate agent is a member of a candidate agent library.

In some embodiments, the method further comprises administering the selected candidate agent into a subject having or at risk of developing a neurodegenerative disease. In some embodiments, the number neuronal synapses in the subject is increased. In some embodiments, the neurodegenerative disease is prevented or treated. In some embodiments, the neurodegenerative disease is Alzheimer's disease or Parkinson's disease. In some embodiments, the Celsr is Celsr3. In some embodiments, the Frizzled is Frizzled3. In some embodiments, the Vangl is Vangl2.

Therapeutic Methods

In another aspect, provided herein are also methods and therapeutic agents for the management, prevention and/or treatment of a neurodegenerative disease (e.g., Alzheimer's disease) resulted from loss of excitatory synapses (e.g., glutamatergic synapses) in the nervous system of a subject. In specific embodiments, the neurodegenerative disease is Alzheimer's disease. In specific embodiments, the neurodegenerative disease is Parkinson's Disease.

In some embodiments, the method of managing, preventing, or treating a neurodegenerative disease in a subject comprises administering to the subject a therapeutic effective amount of (a) a Celsr agonist; (b) Frizzled agonist; (c) Vangl inhibitor; (d) Ryk inhibitor; (e) an Aβ inhibitor; or (f) any combination of (a) to (e).

In specific embodiments, the method of managing, preventing, or treating a neurodegenerative disease in a subject comprises administering a therapeutic effective amount of an Aβ inhibitor that blocks binding of Aβ to Celsr. In some embodiments, Celsr is located at the presynaptic site of a neuron. In some embodiments, Celsr is located on the postsynaptic membrane of a synapse. In some embodiments, Celsr is located at the postsynaptic site of a neuron. In some embodiments, Celsr is located at the postsynaptic membrane of a synapse. In some embodiments, Celsr is located on both the presynaptic and postsynaptic site of a population of neurons. In some embodiments, Celsr is located on both the presynaptic and postsynaptic membranes of a synapse. In particular embodiments, the synapses are excitatory synapses. In particular embodiments, the synapses are glutamatergic synapses.

According to the present disclosure, various known isoforms of Celsr can be the target of Aβ to induce synapse loss. Accordingly, in some embodiments, the Aβ inhibitor specifically binds to one of the Celsr isoforms, thereby blocking binding of Aβ to such Celsr isoform. For example, in some embodiments, the Aβ inhibitor specifically binds to Celsr isoform Celsr 1. In some embodiments, the Aβ inhibitor specifically binds to Celsr isoform Celsr 1. In some embodiments, the Aβ inhibitor specifically binds to Celsr isoform Celsr 3.

In alternative embodiments, the Aβ inhibitor is capable of specifically binding to multiple Celsr isoforms, thereby blocks binding of Aβ to such Celsr isoforms. For example, in some embodiments, the Aβ inhibitor specifically binds to Celsr isoforms Celsr 1 and Celsr2. In some embodiments, the Aβ inhibitor specifically binds to Celsr isoforms Celsr 1 and Celsr 3. In some embodiments, the Aβ inhibitor specifically binds to Celsr isoforms Celsr 2 and Celsr 3.

In particular embodiments, the Aβ inhibitor is capable of specifically binding to multiple Celsr isoforms, and exhibits preferential binding to one isoform over another isoform. In specific embodiments, the Aβ inhibitor preferentially binds to Celsr 3 over Celsr 2. In specific embodiments, the Aβ inhibitor binds to Celsr 3 with a K_{D} less than the K_{D} exhibited for binding with Celsr 2. In specific embodiments, the Aβ inhibitor binds to Celsr 3 with a K_{D} of less than about 95%, less than about 90%, less than about 80%, less than about 70%, less than about 60%, less than about 50%, less than about 40%, less than about 30%, less than about 20%, less than about 10%, or less than about 5% of the K_{D} exhibited for binding with Celsr 2.

In some embodiments, the Aβ inhibitor competes with Aβ for binding to the EGF7, EGF8, and/or Laminin G1 domains of Celsr. In particular embodiments, the Aβ inhibitor competes with Aβ for binding to the EGF7 domain of Celsr. In particular embodiments, the Aβ inhibitor competes with Aβ for binding to the EGF8 domain of Celsr. In particular embodiments, the Aβ inhibitor competes with Aβ for binding to the Laminin G1 domain of Celsr. In particular embodiments, the Aβ inhibitor competes with Aβ for binding to both the EGF7 and EGF8 domains of Celsr. In particular embodiments, the Aβ inhibitor competes with Aβ for binding to both the EGF7 domain and the Laminin G1 domain of Celsr. In particular embodiments, the Aβ inhibitor competes with Aβ for binding to both the EGF8 domain and the Laminin G1 domain of Celsr. In particular embodiments, the Aβ inhibitor competes with Aβ for binding to the EGF7 domain, EGF8 domain and the Laminin G1 domain of Celsr.

In some embodiments, the Aβ is an Aβ peptide in monomeric form. In other embodiments, the Aβ is multiple Aβ peptides aggregated in the oligomeric form. Particularly, according to the present disclosure, different species of Aβ monomers (e.g., having different lengths and/or sequences) can aggregate in the oligomeric form. For example, different species of Aβ monomers can be peptides ranging in size from 37 to 49 amino acid residues, which are produced through the proteolytic processing of the amyloid precursor protein (APP) by β-secretase and γ-secretase. Alternatively, an Aβ oligomer can also contain several Aβ monomers of the same species, such as but not limited to Aβ42 and other species of Aβ monomers known in the art.

In specific embodiments, an Aβ oligomer comprises at least 2 Aβ monomers, such as at least 2, 3, 4, 5, 6, 7, 8, 9, 10, 11, 12, 13, 14, 15, 16, 17, 18, 19 or 20 Aβ monomers. In specific embodiments, an Aβ oligomer comprises about 2 to 8 Aβ monomers. In specific embodiments, an Aβ oligomer comprises about 2 to 6 Aβ monomers. In specific embodiments, an Aβ oligomer comprises about 2 to 4 Aβ monomers. In specific embodiments, an Aβ oligomer comprises 2 Aβ monomers. In specific embodiments, an Aβ oligomer comprises 3 Aβ monomers. In specific embodiments, an Aβ oligomer comprises about 4 Aβ monomers. In any of the embodiments described in this paragraph, the Aβ monomers found in an Aβ oligomer can be of the same or different species. In any of the embodiments described in this paragraph, the Aβ monomer found in an Aβ oligomer is Aβ42.

In some embodiments, the Aβ inhibitor is an anti-Celsr antibody or antigen binding fragment thereof. In some embodiments, the anti-Celsr antibody specifically binds to an epitope in the EGF7 domain of Celsr3. In some embodiments, the anti-Celsr antibody specifically binds to an epitope in the EGF8 domain of Celsr3. In some embodiments, the anti-Celsr antibody specifically binds to an epitope in the Laminin G1 domain of Celsr3.

In some embodiments, the Aβ inhibitor is an anti-Celsr antibody or antigen binding fragment thereof that specifically binds to the Celsr isoform Celsr 1. In some embodiments, the Aβ inhibitor is an anti-Celsr antibody or antigen binding fragment thereof that specifically binds to the Celsr isoform Celsr 2. In some embodiments, the Aβ inhibitor is an anti-Celsr antibody or antigen binding fragment thereof that specifically binds to the Celsr isoform Celsr3.

In alternative embodiments, the Aβ inhibitor is an anti-Celsr antibody or antigen binding fragment thereof that specifically binds to multiple Celsr isoforms selected from Celser 1, Celsr 2 and Celsr 3. In specific embodiments, the Aβ inhibitor is an anti-Celsr antibody or antigen binding fragment thereof that preferentially binds to Celsr 3 over Celsr 2. In specific embodiments, the anti-Celsr antibody or antigen binding fragment thereof binds to Celsr 3 with a K_{D} less than the K_{D} exhibited for binding with Celsr 2. In specific embodiments, the anti-Celsr antibody or antigen binding fragment thereof binds to Celsr 3 with a K_{D} of less than about 95%, less than about 90%, less than about 80%, less than about 70%, less than about 60%, less than about 50%, less than about 40%, less than about 30%, less than about 20%, less than about 10%, or less than about 5% of the K_{D} exhibited for binding with Celsr 2.

In some embodiments, the Aβ inhibitor is an antibody or antigen binding fragment thereof that binds to Aβ, and upon binding to Aβ prevents or reduces Aβ binding to Celsr.

In some embodiments, the method of managing, preventing, or treating a neurodegenerative disease in a subject further comprises administering to the subject at least one additional therapeutic agent. In some embodiments, the at least one additional therapeutic agent is selected from a Ryk inhibitor, a Vangl inhibitor, a Celsr agonist, or a Frizzled agonist, as described herein.

In specific embodiments, the method of managing, preventing, or treating a neurodegenerative disease in a subject comprises administering to the subject a therapeutically effective amount of an Aβ inhibitor as described herein in combination with at least one additional therapeutic agent comprising a Ryk inhibitor.

In some embodiments, the Ryk inhibitor exerts the inhibitory function by reducing the amount of Ryk protein produced by a cell (e.g., a neuron). In some embodiments, the Ryk inhibitor comprises a nucleic acid molecule. In some embodiments, the nucleic acid molecule is a microRNA, siRNA or CRISPR-based gene editing construct that reduce or inhibits expression of Ryk encoding gene.

In some embodiments, the Ryk inhibitor exerts the inhibitory function by preventing Ryk from binding to one or more PCP pathway components present in the synaptic site of a neuron. In particular embodiments, the Ryk inhibitor prevents Ryk from binding to an intracellular complex comprising Celsr and Frizzled in the presynaptic site of a neuron. In particular embodiments, the Ryk inhibitor prevents Ryk from binding to an intracellular complex comprising Celsr and Frizzled in the presynaptic membrane of a synapse. In some embodiments, the synapses are excitatory synapses. In specific embodiments, the synapses are glutamatergic synapses.

In some embodiments, the Ryk inhibitor prevents Ryk from binding to an intercellular complex comprising Celsr proteins located in the presynaptic and postsynaptic sites of neurons. In some embodiments, the Ryk inhibitor prevents Ryk from binding to an intercellular complex comprising Celsr proteins located in the presynaptic and postsynaptic membranes of a synapse. In some embodiments, the synapses are excitatory synapses. In specific embodiments, the synapses are glutamatergic synapses.

In some embodiments, the Ryk inhibitor prevents Ryk from binding to an intercellular complex comprising Celsr proteins located in the presynaptic and postsynaptic sites of a neuron and Frizzled located in the presynaptic site of a neuron. In some embodiments, the Ryk inhibitor prevents Ryk from binding to an intercellular complex comprising Celsr proteins located in the presynaptic and postsynaptic membranes of a synapse and Frizzled located in the presynaptic membrane of the synapse. In some embodiments, the synapses are excitatory synapses. In specific embodiments, the synapses are glutamatergic synapses.

In particular embodiments, the Ryk inhibitor prevents Ryk from binding to Frizzled in the presynaptic site of a neuron. In particular embodiments, the Ryk inhibitor prevents Ryk from binding to Frizzled in the presynaptic membrane of a synapse. In some embodiments, the synapses are excitatory synapses. In specific embodiments, the synapses are glutamatergic synapses.

In some embodiments, the Ryk inhibitor prevents Ryk from binding to Wnt. In some embodiments, the synapses are excitatory synapses. In specific embodiments, the synapses are glutamatergic synapses.

In some embodiments, the Ryk inhibitor is an antagonistic antibody specifically binding to Ryk, or a molecule comprising the antigen binding fragment of the anti-Ryk antibody. In some embodiments, the Ryk inhibitor comprises one or more anti-Ryk antibodies or antigen binding fragment thereof as described in International Application No.: PCT/US2017/024494 (Published as WO 2017/172733). In some embodiments, the Ryk inhibitor is a small molecule compound.

In specific embodiments, the method of managing, preventing, or treating a neurodegenerative disease in a subject comprises administering to the subject a therapeutically effective amount of an Aβ inhibitor as described herein in combination with at least one additional therapeutic agent comprising a Vangl inhibitor.

In some embodiments, the Vangl inhibitor exerts the inhibitory function by reducing the amount of Vangl protein produced by a cell. In some embodiments, the Vangl inhibitor comprises a nucleic acid molecule. In some embodiments, the nucleic acid molecule is a microRNA, siRNA or CRISPR-based gene editing construct that reduce or inhibits expression of Vangl encoding gene.

In some embodiments, the Vangl inhibitor exerts the inhibitory function by preventing Vangl from binding to one or more PCP pathway components present in the synaptic site of a neuron. In particular embodiments, the Vangl inhibitor prevents Vangl from binding to an intracellular complex comprising Celsr and Frizzled in the presynaptic site of a neuron. In particular embodiments, the Vangl inhibitor prevents Vangl from binding to an intracellular complex comprising Celsr and Frizzled located in the presynaptic membrane of a synapse. In some embodiments, the Vangl inhibitor prevents Vangl from binding to an intercellular complex comprising Celsr proteins located in the presynaptic and postsynaptic sites of neurons. In some embodiments, the Vangl inhibitor prevents Vangl from binding to an intercellular complex comprising Celsr proteins located in the presynaptic and postsynaptic membranes of a synapse. In some embodiments, the synapses are excitatory synapses. In specific embodiments, the synapses are glutamatergic synapses.

In some embodiments, the Vangl inhibitor prevents Vangl from binding to an intercellular complex comprising Celsr proteins located in the presynaptic and postsynaptic sites of a neuron and Frizzled located in the presynaptic site of a neuron. In some embodiments, the Vangl inhibitor prevents Vangl from binding to an intercellular complex comprising Celsr proteins located in the presynaptic and postsynaptic membranes of a synapse and Frizzled located in the presynaptic membrane of the synapse. In some embodiments, the synapses are excitatory synapses. In specific embodiments, the synapses are glutamatergic synapses.

In particular embodiments, the Vangl inhibitor prevents Vangl from binding to Frizzled in the presynaptic site of a neuron. In some embodiments, the synapses are excitatory synapses. In specific embodiments, the synapses are glutamatergic synapses. In particular embodiments, the Vangl inhibitor prevents Vangl from binding to Frizzled in the presynaptic membrane of a neuron. In some embodiments, the synapses are excitatory synapses. In specific embodiments, the synapses are glutamatergic synapses.

In particular embodiments, the Vangl inhibitor prevents Vangl located in the postsynaptic site from binding to an intracellular complex comprising Celsr and Frizzled in the presynaptic site of a neuron. In particular embodiments, the Vangl inhibitor prevents Vangl located in the postsynaptic membrane of a synapse from binding to an intracellular complex comprising Celsr and Frizzled in the presynaptic membrane of the synapse. In some embodiments, the synapses are excitatory synapses. In specific embodiments, the synapses are glutamatergic synapses.

In some embodiments, the Vangl inhibitor prevents Vangl located in the postsynaptic site from binding to an intercellular complex comprising Celsr proteins located in the presynaptic and postsynaptic sites of neurons. In some embodiments, the Vangl inhibitor prevents Vangl located in the postsynaptic membrane of a synapse from binding to an intercellular complex comprising Celsr proteins located in the presynaptic and postsynaptic membranes of the synapse. In some embodiments, the synapses are excitatory synapses. In specific embodiments, the synapses are glutamatergic synapses.

In some embodiments, the Vangl inhibitor prevents Vangl located in the postsynaptic site from binding to an intercellular complex comprising Celsr proteins located in the presynaptic and postsynaptic sites of a neuron and Frizzled located in the presynaptic site of a neuron. In some embodiments, the Vangl inhibitor prevents Vangl located in the postsynaptic membrane of a synapse from binding to an intercellular complex comprising Celsr proteins located in the presynaptic and postsynaptic membranes of the synapse and Frizzled located in the presynaptic membrane of the synapse. In some embodiments, the synapses are excitatory synapses. In specific embodiments, the synapses are glutamatergic synapses.

In particular embodiments, the Vangl inhibitor prevents Vangl located in the postsynaptic site from binding to Frizzled in the presynaptic site of a neuron. In particular embodiments, the Vangl inhibitor prevents Vangl located in the postsynaptic membrane of a synapse from binding to Frizzled in the presynaptic membrane of the synapse. In some embodiments, the synapses are excitatory synapses. In specific embodiments, the synapses are glutamatergic synapses.

In some embodiments, the Vangl inhibitor exerts the inhibitory function by preventing Vangl from disrupting intercellular complex formed at synapses by one or more PCP pathway components. In particular embodiments, the Vangl inhibitor prevents Vangl from disrupting an intercellular complex comprising Celsr proteins located in the presynaptic and postsynaptic sites of neurons. In some embodiments, the Vangl inhibitor prevents Vangl from disrupting an intercellular complex comprising Celsr proteins located in the presynaptic and postsynaptic membranes of a synapse. In some embodiments, the synapses are excitatory synapses. In specific embodiments, the synapses are glutamatergic synapses.

In some embodiments, the Vangl inhibitor exerts the inhibitory function by preventing Vangl from disrupting an intracellular complex formed by one or more PCP pathway components. In particular embodiments, the Vangl inhibitor prevents Vangl from disrupting an intracellular complex comprising Celsr and Frizzled in the presynaptic site of a neuron. In particular embodiments, the Vangl inhibitor prevents Vangl from disrupting an intracellular complex comprising Celsr and Frizzled located in the presynaptic membrane of a synapse. In some embodiments, the synapses are excitatory synapses. In specific embodiments, the synapses are glutamatergic synapses.

In some embodiments, the Vangl inhibitor is an antagonistic antibody specifically binding to Vangl, or a molecule comprising the antigen binding fragment of the anti-Vangl antibody. In some embodiments, the Vangl inhibitor is a small molecule compound.

In specific embodiments, the method of managing, preventing, or treating a neurodegenerative disease in a subject comprises administering to the subject a therapeutically effective amount of an Aβ inhibitor as described herein in combination with at least one additional therapeutic agent comprising a Celsr agonist.

In some embodiments, the Celsr agonist exerts the agonistic activity by increasing the amount of Celsr protein produced by a cell (e.g., a neuron).

In some embodiments, the Celsr agonist exerts the agonistic activity by reducing endocytosis of Celsr located in the synaptic site of a neuron. In some embodiments, the Celsr agonist exerts the agonistic activity by reducing endocytosis of Celsr located in the presynaptic membrane of a neuron. In some embodiments, the Celsr agonist exerts the agonistic activity by reducing endocytosis of Celsr located in the postsynaptic membrane of a neuron. In some embodiments, the Celsr agonist exerts the agonistic activity by reducing endocytosis of Celsr located in both the presynaptic membrane and postsynaptic membranes of a population of neurons. In some embodiments, the synapses are excitatory synapses. In specific embodiments, the synapses are glutamatergic synapses.

In some embodiments, the Celsr agonist exerts the agonistic activity by increasing transportation of Celsr to the synaptic site of a neuron. In some embodiments, the Celsr agonist exerts the agonistic activity by increasing assembly of Celsr into the presynaptic membrane of a neuron. In some embodiments, the Celsr agonist exerts the agonistic activity by increasing assembly of Celsr into the postsynaptic membrane of a neuron. In some embodiments, the Celsr agonist exerts the agonistic activity by increasing assembly of Celsr into both the presynaptic membrane and postsynaptic membranes of a population of neurons. In particular embodiments, the synapses are excitatory synapses. In particular embodiments, the synapses are glutamatergic synapses.

In specific embodiments, the method of managing, preventing, or treating a neurodegenerative disease in a subject comprises administering to the subject a therapeutically effective amount of an Aβ inhibitor as described herein in combination with at least one additional therapeutic agent comprising a Frizzled agonist.

In some embodiments, the Frizzled agonist exerts the agonistic activity by increasing the amount of Frizzled protein produced by a cell (e.g., a neuron).

In some embodiments, the Frizzled agonist exerts the agonistic activity by reducing endocytosis of Frizzled located in the synaptic site of a neuron. In some embodiments, the Frizzled agonist exerts the agonistic activity by reducing endocytosis of Frizzled located in the presynaptic membrane of a neuron. In some embodiments, the Frizzled agonist exerts the agonistic activity by reducing endocytosis of Frizzled located in the postsynaptic membrane of a neuron. In some embodiments, the Frizzled agonist exerts the agonistic activity by reducing endocytosis of Frizzled located in both the presynaptic membrane and postsynaptic membranes of a population of neurons. In some embodiments, the synapses are excitatory synapses. In specific embodiments, the synapses are glutamatergic synapses.

In some embodiments, the Frizzled agonist exerts the agonistic activity by increasing transportation of Frizzled to the synaptic site of a neuron. In some embodiments, the Frizzled agonist exerts the agonistic activity by increasing assembly of Frizzled into the presynaptic membrane of a neuron. In some embodiments, the Frizzled agonist exerts the agonistic activity by increasing assembly of Frizzled into the postsynaptic membrane of a neuron. In some embodiments, the Frizzled agonist exerts the agonistic activity by increasing assembly of Frizzled into both the presynaptic membranes and postsynaptic membranes of a population of neurons. In particular embodiments, the synapses are excitatory synapses. In particular embodiments, the synapses are glutamatergic synapses.

### EXAMPLES

The examples in this section are offered by way of illustration, and not by way of limitation.

General Methods.

Animals. All animal work in the following studies was approved by the University of California, San Diego (UCSD) Institutional Animal Care and Use Committee. Animals were housed on a 12 hours light/dark cycle and behavioral analyses were done at consistent morning hours during the light cycle. The 5×FAD transgenic mice carrying the following five mutations: Swedish (K670N and M671L), Florida (1716V) and London (V7171) in human APP695 and human PS1 cDNA (M146L and L286V) under the transcriptional control of the neuron-specific Thy-1 promoter and were purchased from The Jackson Laboratory. 5×FAD mice were crossed with Vangl2^{fl/fl} (cKO), which were provided by Yingzi Yang, Harvard Medical School (Song, H. et al. Planar cell polarity breaks bilateral symmetry by controlling ciliary positioning. Nature 466, 378-382, doi:10.1038/nature09129 (2010)).

Aβ oligomer preparation. Human Aβ42 (AnaSpec) or human biotin-beta-Amyloid (1-42) (AnaSpec) was dissolved in dimethyl sulfoxide (DMSO). It was then sonicated and diluted with F12 medium for Aβ monomerization to a concentration of 100 μM. For oligomerization, the solution was incubated for 24-26 hours at 4° C., centrifuged at 16,000×g for 20 min, and the supernatant was collected as oligomerized Aβ.

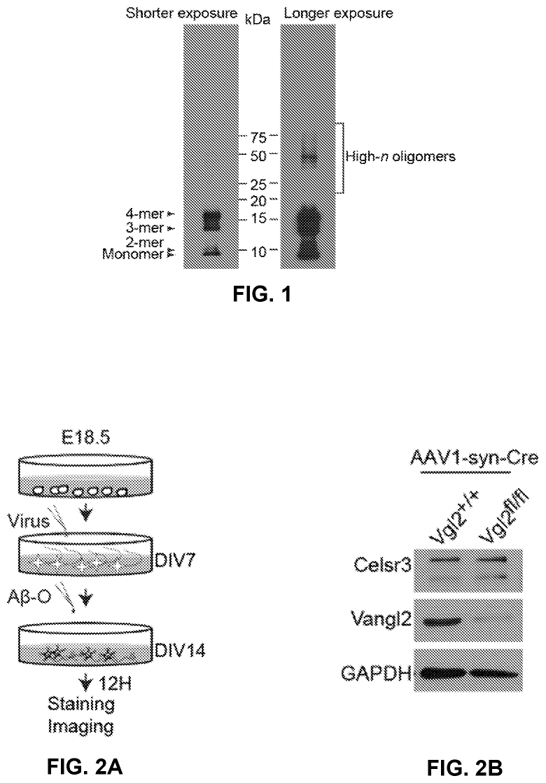

The oligomerized Aβ42 preparations were analyzed via SDS-PAGE using 12% tris-glycine gels. 50 μg Aβ42 peptides were loaded into gels and electrophoretically separated at 25 mA. Gels were transferred onto PVDF membrane. Signals were detected using antibody 6E10 (BioLegend). It was estimated that for FIG. 1 the percentage of Aβ42 present as monomers (defined as molecular weight 2.5-6.5 kD) was 26.94±7.43% (n=4); the percentage as dimer (MW 6.5-11.5 kD) was 2.06±0.41%; the percentage as trimmers (MW 11.5-15.5 kD) was 17.84±0.97%; the percentage as tetramers (MW 15.5-20.5 kD) was 38.15±5.09% and the percentages as high-n oligomers were 15.01±7.95%.

Intracerebroventricular (ICV) injection of Aβ oligomer. Adult (2-3 months old) mice were deeply anaesthetized with an intraperitoneal injection of ketamine/xylazine cocktail until unresponsive to toe and tail pinch. Aβ oligomers (5 ng; volume 250 nl) or PBS (volume 250 nl) was stereotaxically injected into bilateral ventricles (−0.1 mm anteroposterior, 1 mm mediolateral and −2.5 mm dorsoventral). 5 days after ICV injection, brains were harvested for immunohistochemistry.

AAV Cre intrahippocampal injection. Adult Vangl2 cKO and littermates WT controls (2-3 months old) were deeply anaesthetized with an intraperitoneal injection of ketamine/xylazine cocktail until unresponsive to toe and tail pinch. AAV1-hSyn-eGFP-Cre (Addgene) was stereotaxically injected into bilateral hippocampal CA1 (160n1 per site). 2 weeks after the viral injection, mice were stereotaxically injected with Aβ oligomer intraventricularly as described above.

Hippocampal neuron culture. Hippocampi were dissected from E18.5 mice, and hippocampal neuron culture was performed as previously described (Thakar, S. et al. Evidence for opposing roles of Celsr3 and Vangl2 in glutamatergic synapse formation. Proc Natl Acad Sci USA 114, E610-E618, doi:10.1073/pnas.1612062114 (2017)). Briefly, cells ells were pelleted and resuspended in Neurobasal medium supplemented with 1% B27 (Invitrogen), penicillin/streptomycin (CellGro), and Gluta-MAX (Invitrogen) and plated on poly-D-lysine- (Millipore) coated glass coverslips in a 24-well plate at a density of 2 Å˜10⁴ cells per square centimeter for immunostaining. Medium was changed every 4 days. Cultures were grown for 14 to 15 DIV at 37° C. with 5% carbon dioxide.

Hippocampal culture immunofluorescence and image analysis. For synaptic puncta density analysis in cultured hippocampal neurons, neurons on DIV 14 were fixed for 20 min in 4% PFA. After fixation, cells were incubated in a blocking solution (1% bovine serum albumin, and 5% goat serum in Tris buffer saline solution (TBS) with 0.1% Triton X-100) for 1 h, and then stained overnight at 4° C. with primary antibodies chicken anti-MAP2 (neuronal marker; Abcam), guinea pig anti-Bassoon (presynaptic marker; Synaptic Systems) and goat anti-PSD-95 (postsynaptic marker; Millipore). After, cells were incubated with fluorochrome-conjugated secondary antibodies Alexa 488 anti-chicken, Alexa 647 anti-guinea pig and Alexa 568 anti-goat solution for 2 h at room temperature and mounted in mounting media. Z-stacked images were obtained with a Carl Zeiss microscope using a 63× oil-immersion objective. Three or more neurons with pyramidal morphology and at least two diameters' distance from the neighboring neurons were selected per coverslip. Three coverslips were used for each group per experiment. Secondary dendrites were chosen for puncta analysis. Number of puncta was analyzed using the ImageJ Synapse Counter plug-in and the length of the dendrite was analyzed by ImageJ (NIH).

Immunofluorescence staining. For in vivo synaptic protein immunostaining, mice were deeply anesthetized with an intraperitoneal injection of ketamine/xylazine until unresponsive to toe and tail pinch and perfused with PBS followed by 4% PFA. Brains were removed and postfixed in 4% PFA overnight at 4° C. After, brains were cryoprotected in 30% sucrose for 2 days and coronal free-floating sections were prepared at 30 μm in a vibratome. The sections obtained were treated with 1% SDS for 5 min at room temperature for antigen retrieval, incubated in a blocking solution (1% bovine serum albumin, and 5% goat serum in Tris buffer saline solution (TBS) with 0.1% Triton X-100) for 1.5 h, and then stained overnight at 4° C. with primary antibodies guinea pig anti-Bassoon (presynaptic marker; Synaptic Systems) and goat anti-PSD-95 (postsynaptic marker; Millipore). After, sections were incubated with fluorochrome-conjugated secondary antibodies Alexa 647 anti-guinea pig and Alexa 568 anti-goat solution for 2 hours at room temperature, counterstained with DAPI and mounted in mounting media. The synapses formed between the Schaffer collaterals and the hippocampal CA1 pyramidal neuron apical dendrites spanning the mouse stratum radiatum were imaged. Fluorescent z-stack images were acquired with an LSM510 Zeiss confocal microscope using a 63× oil-immersion objective with 2× zoom-in. Number of puncta were analyzed using the ImageJ Synapse Counter plug-in.

Plasmid, inhibitors and antibodies. Celsr3-Flag, Fzd3-HA, Vangl2-Myc and tdTomato expressing constructs were described previously (Shafer, B., Onishi, K., Lo, C., Colakoglu, G. & Zou, Y. Vangl2 promotes Wnt/planar cell polarity-like signaling by antagonizing Dvl1-mediated feedback inhibition in growth cone guidance. Dev Cell 20, 177-191, doi:S1534-5807(11)00003-7 [pii] 10.1016/j.devcel.2011.01.002 (2011); Onishi, K. et al. Antagonistic Functions of Dishevelleds Regulate Frizzled3 Endocytosis via Filopodia Tips in Wnt-Mediated Growth Cone Guidance. J Neurosci 33, 19071-19085, doi:33/49/19071 [pii]10.1523/JNEUROSCI.2800-13.2013 (2013).). Recombinant Wnt5a was purchased from R&D, Sulfo-NHS-LC-Biotin was purchased from Pierce. The antibodies used in this study include α-Vangl2 (Santa Cruz), α-Celsr3 (Rabbit polyclonal antibodies were generated by the Zou lab), α-Flag (Sigma), α-GAPDH (Chemicon), α-Insulin Rβ (Santa Cruz) and α-HA (Covance).

To generate truncated Celsr3 constructs, full-length Celsr3 extracellular domain is amplified by PCR, digested with EcoRV/NheI, and subcloned into pCAGEN vector using primers as follows:

ΔEGF/Lam_Celsr3 Forward primer 1:
(SEQ ID NO: 1)
5′-GATCGATATCTTCTCTGGAGAGCTCACAGC-3′ .
ΔEGF/Lam_Celsr3 Reverse primer 1:
(SEQ ID NO: 2)
5′-GCAGGCATCGTAAAAGGGCAGCACGTCGAG-3′ .
ΔEGF/Lam_Celsr3 Forward primer 2:
(SEQ ID NO: 3)
5‘-GTGCTGCCCTTTTACGATGCCTGCCCCAAG-3 .
ΔEGF/Lam_Celsr3 Forward primer:
(SEQ ID NO: 4)
5′-GATCGCTAGCAAGTAGGCCAGCAAG-3′ .
ΔEGFl_Celsr3 Forward primer:
(SEQ ID NO: 5)
5‘-TGCTGCCCTTTACAGAGCTCGACCTCTGTTAC-3 .
ΔEGFl_Celsr3 Reverse primer:
(SEQ ID NO: 6)
5′-CGAGCTCTGTAAAGGGCAGCACGTCGAG-3′ .
ΔEGF2_Celsr3 Forward primer:
(SEQ ID NO: 7)
5′-TCTGTGAGACACTGGACACTGAAGCTGGACG-3′ .
ΔEGF2_Celsr3 Reverse primer:
(SEQ ID NO: 8)
5′-TCAGTGTCCAGTGTCTCACAAGAAGTCTCCCG-3′ .
ΔEGF3_Celsr3 Forward primer:
(SEQ ID NO: 9)
5′-GCTGGACACTGTGGCCGCACGCTCCTTTC-3′ .
ΔEGF3_Celsr3 Reverse primer:
(SEQ ID NO: 10)
5′-GTGCGGCCACAGTGTCCAGCTCGCAGTC-3′ .
ΔLaminin Gl_Celsr3 Forward primer:
(SEQ ID NO: 11)
5′-ACGCTGTGAGCAGGCCAAGTCACACTTTTGTG-3′
[ΔLaminin Gl_Celsr3 Reverse primer:
(SEQ ID NO: 12)
5‘-ACTTGGCCTGCTCACAGCGTGGACCATC-3 .
ΔEGF4_Celsr3 Forward primer:
(SEQ ID NO: 13)
5′-AGGCTGCCAGCTCACAATGGCCCATCCCTAC-3′ .
ΔEGF4_Celsr3 Reverse primer:
(SEQ ID NO: 14)
5′-CCATTGTGAGCTGGCAGCCTGCCATAGTG-3′ .
ΔLaminin G2_Celsr3 Forward primer:
(SEQ ID NO: 15)
5′-CTGTCGACTCACTGTGACCAACCCCTGTG-3′ .
ΔLaminin G2_Celsr3 Reverse primer:
(SEQ ID NO: 16)
5‘-TGGTCACAGTGAGTCGACAGTCTTTGCCACC-3‘ .
ΔEGF5_Celsr3 Forward primer:
(SEQ ID NO: 17)
5′-TGGCTGTACTGATGCCTGCCTCCTGAACC-3′ .
ΔEGF5_Celsr3 Reverse primer:
(SEQ ID NO: 18)
5′-GGCAGGCATCAGTACAGCCAGGCTCCACATTC-3′ .
ΔEGF6_Celsr3 Forward primer:
(SEQ ID NO: 19)
5′-AGGCTGTGTGTATTTTGGTCAGCACTGTGAGCAC-3′
ΔEGF6_Celsr3 Reverse primer:
(SEQ ID NO: 20)
5′-GCTGACCAAAATACACACAGCCTGGGCCATAG-3′ .
ΔEGF7_Celsr3 Forward primer:
(SEQ ID NO: 21)
5′-TGTGAGTGGCAAGACGAATGGCCAGTGCC-3′ .
ΔEGF7_Celsr3 Reverse primer:
(SEQ ID NO: 22)
5‘-CCATTCGTCTTGCCACTCACAGTCACAAG-3* .
ΔEGF8_Celsr3 Forward primer:
(SEQ ID NO: 23)
5-CAACTGCAACCCCCACAGCGGGCAGTG-3′ .
ΔEGF8_Celsr3 Reverse primer:
(SEQ ID NO: 24)
5′-CTGTGGGGGTTGCAGTTGGGGTCAAAGC-3′ .
ΔLaminin EGF_Celsr3 Reverse primer:
(SEQ ID NO: 25)
5′-GCATCGTAGAGTGGGAGGCATGAGTCACTG-3′ .
ΔLaminin EGF_Celsr3 Forward primer:
(SEQ ID NO: 26)
5′-ATGCCACCCACTCTACGATGCCTGCCCCAAG-3′ .

HEK293T cell transfection. HEK293T cells were purchased from ATCC and maintained in Dulbecco's modified Eagle's medium (DMEM) containing 10% fetal bovine serum. Transfection of HEK293T cells was carried out using 1 mg/ml Polyethyleneimine MAX (Polyscience). Mycoplasma contamination was monitored by DAPI staining.

Binding assay. HEK293 cells were transiently transfected (polyethylenimine) with expression vectors encoding TdTomato, Celsr3-Flag or control empty vectors (pCAGEN). Two days post-transfection, cells were treated with biotinylated Aβ oligomer for 2 h at 37° C., washed twice, and fixed with 4% PFA for 20 min, blocked with 5% donkey serum in PBS with 0.1% Triton X-100. The bound Aβ peptides were visualized with streptavidin-Alexa fluorophore conjugates (Alexa 488). DAPI was used to counterstain cell nuclei; TdTomato was used to monitor construct transfection. Anti-flag antibody was used to stain Celsr3. Fluorescent images were captured with Zeiss LSM 880 fast airyscan using a 63× oil-immersion objective.

Surface biotinylation assay. The surface biotinylation and NeutrAvidin pull down methods have been described previously (Shafer, B., Onishi, K., Lo, C., Colakoglu, G. & Zou, Y. Vangl2 promotes Wnt/planar cell polarity-like signaling by antagonizing Dvl1-mediated feedback inhibition in growth cone guidance. Dev Cell 20, 177-191, doi:S1534-5807(11)00003-7 [pii] 10.1016/j.devcel.2011.01.002 (2011); Onishi, K. et al. Antagonistic Functions of Dishevelleds Regulate Frizzled3 Endocytosis via Filopodia Tips in Wnt-Mediated Growth Cone Guidance. J Neurosci 33, 19071-19085, doi:33/49/19071 [pii]10.1523/JNEUROSCI.2800-13.2013 (2013)). Briefly, 48 hr after transfection with indicated plasmids, HEK293T cells (seeded on 20 μg/ml PDL-coated six-well plate) cells were washed with ice-cold PBS (pH 8.0) three times and incubated with 1 mg/ml Sulfo-NHS-LC-Biotin (ThermoFisher Scientific)/PBS for 2 min at room temperature to initiate the reaction, followed by incubation on ice for 1 hr. After quenching active biotin by washing with ice-cold 100 mM Glycine/PBS twice followed by normal ice-cold PBS, the cell lysates were incubated with NeutrAvidin agarose for 2 hr and then precipitated. For quantification, three independent experiments were performed, and the band intensity was quantified with ImageJ (NIH).

Coimmunoprecipitation. 48 hr after transfection with the indicated plasmids, HEK293T cells were lysed with IP buffer (20 mM Tris HCl (pH 7.5), 150 mM NaCl, 1 mM EGTA, 5 mM NaF, 10 mM β-glycerophosphate, 1 mM Na₃VO₄, 1 mM DTT and protease inhibitor cocktail (SIGMA), 0.1% TX-100). Lysates were immunoprecipitated with anti-HA, anti-Myc or anti-Flag antibodies and with protein A/G agarose (Santa Cruz). Experiments were repeated three times and showed similar results.

Statistical analysis. Comparisons between multiple experimental groups were performed by one-way ANOVA followed by Tukey-Kramer post-hoc test, when appropriate. Comparisons between two experimental groups were performed by Student's t test. All statistical analyses were performed using GraphPad Prism software (La Jolla, Calif., USA). A value of p<0.05 was considered significant.

### Example 1

### Vangl is Involved in Aβ Oligomer-Induced Synapse Loss

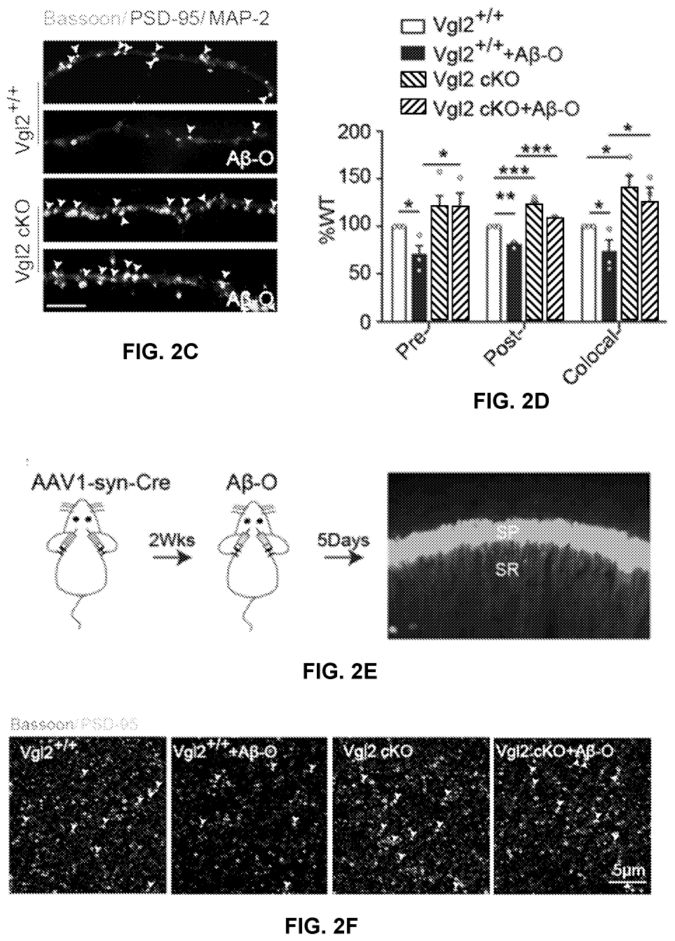

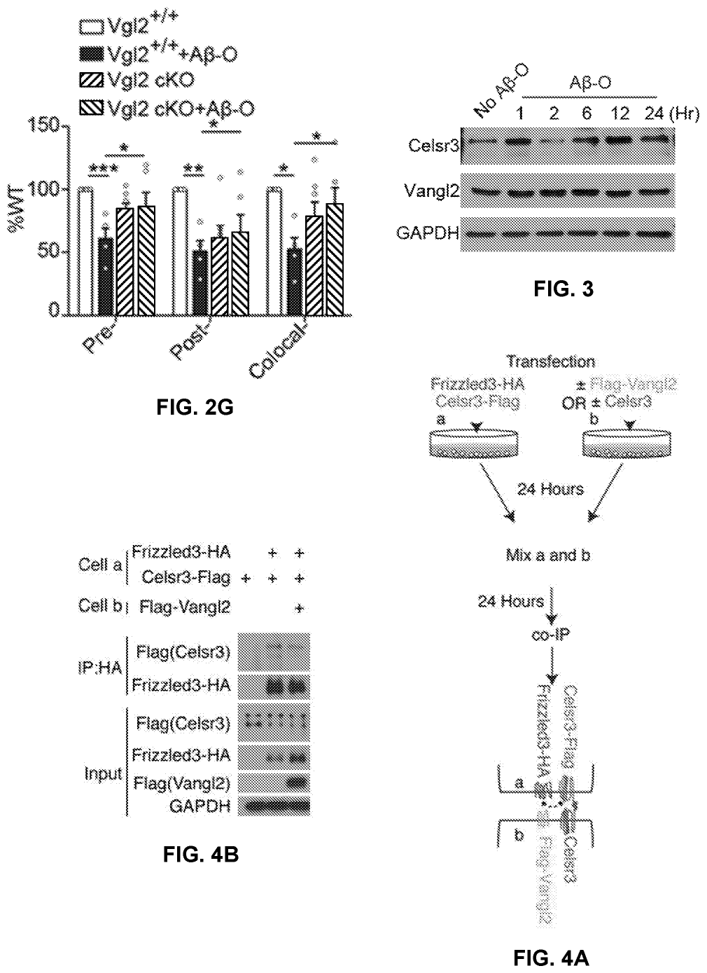

To examine the role of PCP pathway on Aβ oligomer-induced synaptotoxicity, first examined was hippocampal neurons in 14-DIV cultures prepared from embryonic day (E) 18.5 Vangl2 cKO mice. Adeno-associated virus (AAV) that harbors the human synapsin (hSyn) promotor with CMV enhancer was added into the medium on DIV-7 and 400 nM monomer equivalent of Aβ oligomers (FIG. 1, effective concentration of dimer is 80 nM and tetramer is ˜152 nM as calculated in the Methods), which is commonly used (Lacor, P. N. et al. Abeta oligomer-induced aberrations in synapse composition, shape, and density provide a molecular basis for loss of connectivity in Alzheimer's disease. J Neurosci 27, 796-807, doi:10.1523/JNEUROSCI.3501-06.2007 (2007); Lauren, J., Gimbel, D. A., Nygaard, H. B., Gilbert, J. W. & Strittmatter, S. M. Cellular prion protein mediates impairment of synaptic plasticity by amyloid-beta oligomers. Nature 457, 1128-1132, doi:10.1038/nature07761 (2009)), was added on DIV-14 for 12 hours (FIGS. 2A and 2B). Littermate wild type neurons (Vangl2^{+/+}, WT) were also treated with AAV-Cre as controls. It was found that Aβ oligomers induced 30% fewer presynaptic puncta as revealed by bassoon staining, 20% fewer postsynaptic puncta as shown by PSD-95 staining, and 30% fewer colocalized puncta characteristic of glutamatergic synapse in WT mice. However, the number of glutamatergic synapses in Vangl2 cKO mice was unchanged with the same treatment of Aβ oligomers (FIGS. 2C and 2D). Compared with WT, Vangl2 cKO cultures contained 20% more presynaptic puncta, 22% more postsynaptic puncta and 40% more colocalized puncta characteristic of glutamatergic synapses in the absence of Aβ oligomer, consistent with the finding that Vangl2 inhibits synapse formation (FIGS. 2C and 2D). The proteins of Vangl2 and Celsr3 showed no change after Aβ oligomers challenging (FIG. 3), suggesting that Aβ oligomers do not exert the effects by regulating the levels of these proteins.

To examine the function of Vangl2 on Aβ oligomers induced synaptotoxicity in vivo, the same AAV-Cre was injected into the hippocampal CA1 region of WT and of Vangl2 floxed allele for 2 weeks, and then 5 ng of Aβ oligomers were injected into ventricular zone bilaterally (Hong, S. et al. Complement and microglia mediate early synapse loss in Alzheimer mouse models. Science 352, 712-716, doi:10.1126/science.aad8373 (2016)) (FIG. 2E). The study focused on the glutamatergic synapses formed between the Schaffer collaterals and hippocampal CA1 pyramidal neuron apical dendrites spanning the mouse stratum radiatum, which is a commonly used model for studying synapse formation and showed a region-specific synapse loss in Aβ patients. 5 days after the Aβ oligomers injection, a significant loss of synapses was observed in Aβ oligomers injected WT, but not in Aβ oligomers injected Vangl2 cKO mice. Compared with WT, Vangl2 cKO revealed no significant changes in synapse numbers in the absence of Aβ oligomers (FIGS. 2F and 2G), compared to the cultures using embryonic neurons (FIGS. 2C and 2D). It is possible that synapse turnover is not as rapid in adulthood such that synapse number does not show obvious changes in 2-3 weeks in Vangl2 cKO.

### Example 2-1

### Vangl Disrupts Intercellular Complexes of Planar Cell Polarity (PCP) Signaling

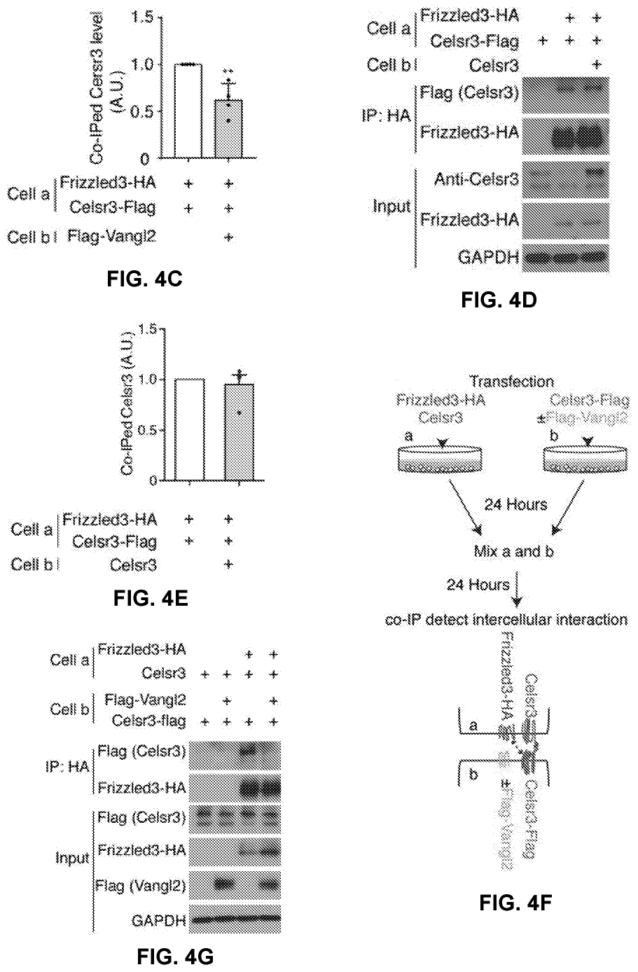

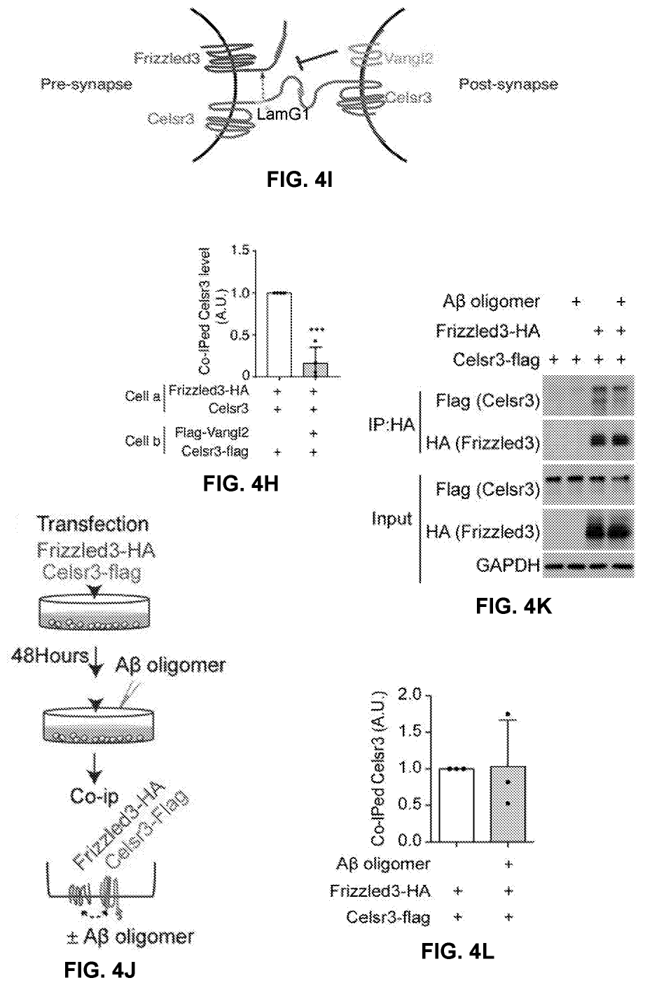

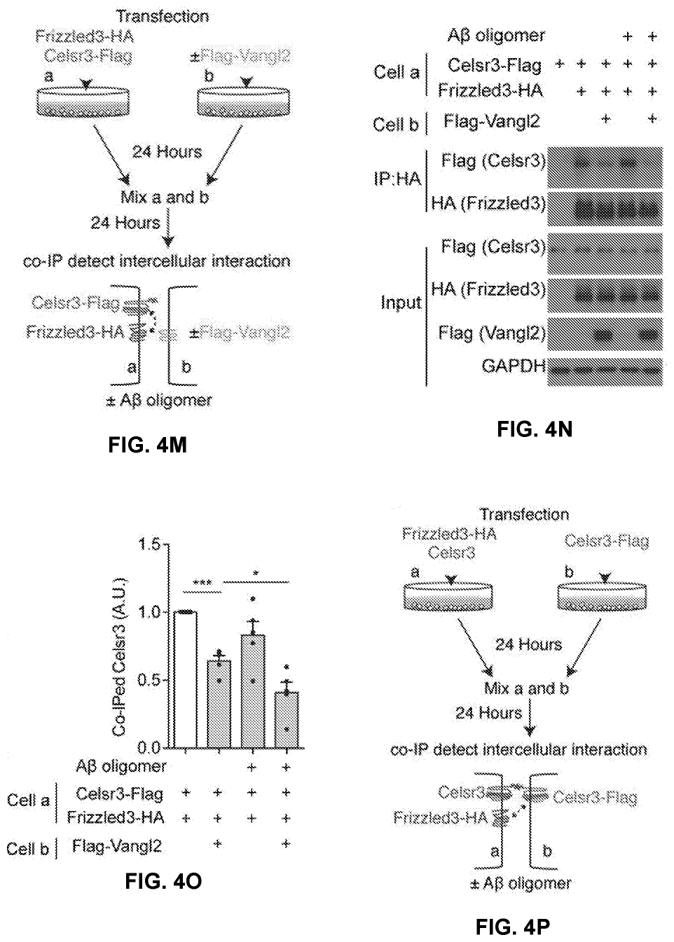

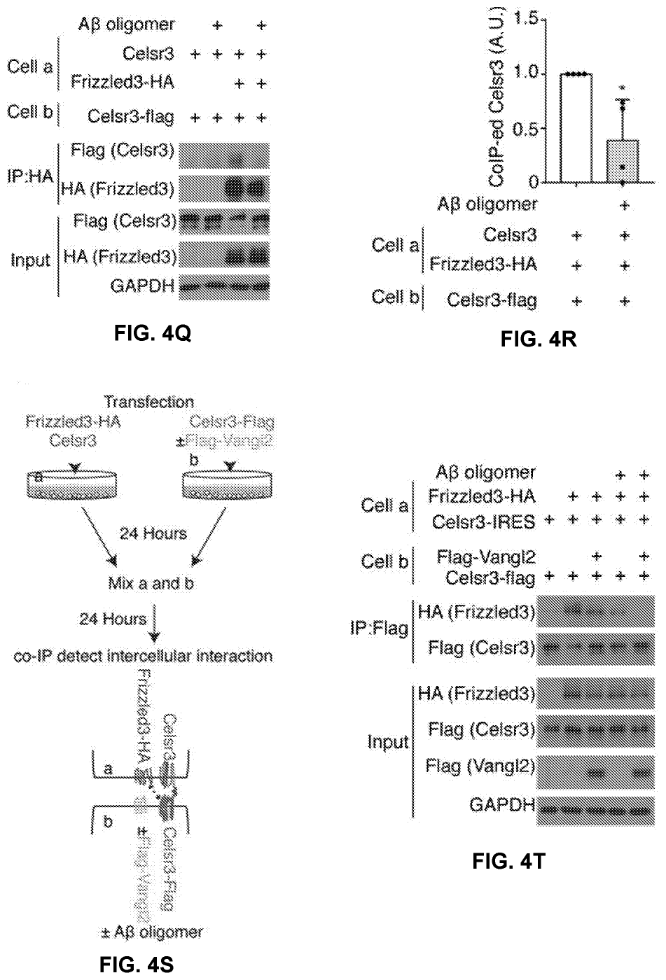

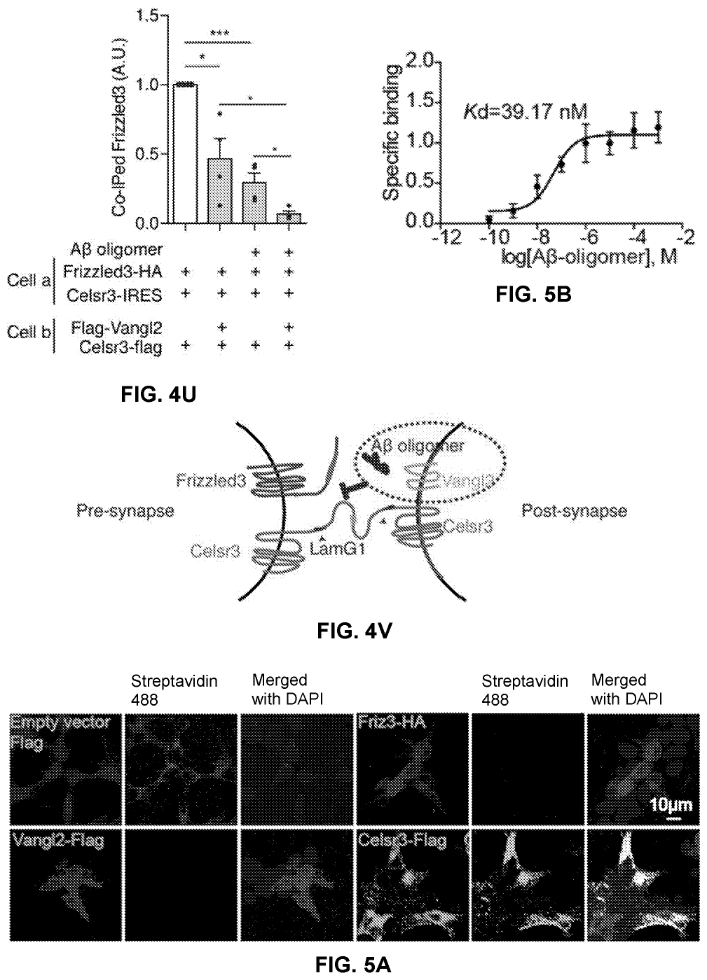

It was discovered that Vangl2 promotes Frizzled3 endocytosis, which forms a complex with Celsr3 on the plasma membrane in PCP signaling. To examine how Vangl2 negatively regulates synapse numbers, an assay was established to test the intercellular PCP complexes, which are known to involve in PCP signaling. Frizzled3 (HA-tagged) and Celsr3 (Flag-tagged) in one dish of HEK293T cells and transfected Vangl2 in another. After culturing them separately for one day, they were mixed together and cultured for one more day and then performed coimmunoprecipitation to test protein-protein interactions (FIG. 4A). It was found that the presence of Vangl2 from the neighboring cell caused the reduction of the interaction between Frizzled3 and Celsr3 by 30-40% (FIGS. 4B and 4C). Celsr3 forms intercellular bridges essential in PCP signaling and synapse formation. It was found that Celsr3 from neighboring cell does not affect the complex between Frizzled3 and Celsr3 (FIGS. 4D and 4E).

Finally, to examine whether Vangl2 disrupts the intercellular bridge, Frizzled3 was pulled down and it was tested how much Celsr3 from the neighboring cell was coimmunoprecipitated. Vangl2 and Celsr3 (Flag-tagged) were transfected in one dish and one day later mixed with cells transfected with Frizzled3 (HA-tagged) and Celsr3 (untagged) (FIG. 4F). It was found that Vangl2 disrupted this intercellular complex as much less Flag-tagged Celsr3 was pulled down by HA-tagged Frizzled3 (FIGS. 4G and 4H). This biochemical function of Vangl2 may be part of the antagonistic interactions against Frizzled3 in PCP signaling (FIG. 4G). Because having Celsr3 in the neighboring cell does not affect the complex between Frizzled3 and Celsr3 (FIGS. 4D and 4E), we believe Vangl2 is the negative regulator of this entire intercellular complex (FIG. 4G).

### Example 2-2

### Aβ Oligomers Enhances Vangl2's Function to Disrupt the Intercellular Complex

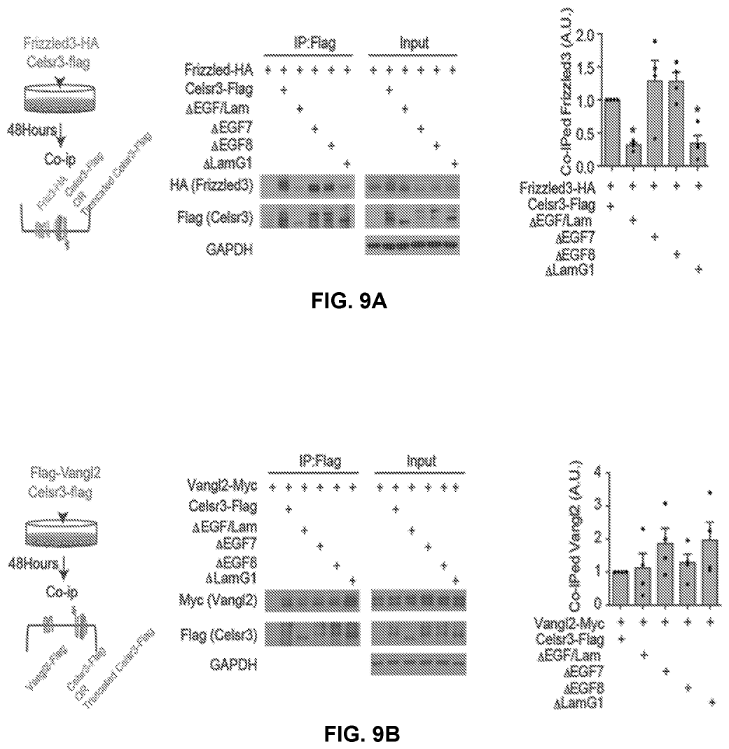

In order to determine how Aβ oligomers lead to synapse loss, another series of biochemical studies were performed to test whether and how Aβ oligomers promote the function of Vangl2. First, it was found Aβ oligomers did not disrupt the interaction between Celsr3 and Frizzled3 that were transfected and expressed in the same cell, suggesting that Aβ oligomers are not sufficient to disrupt the Ceslr3-Frizzled3 complex (FIG. 4J). As shown, Vangl2 expressed in neighboring cell can slightly decrease the interaction between Celsr3 and Frizzled 3 (FIG. 9A). However, the interactions between Frizzled3 and Celsr3 were reduced to a greater extent when Aβ oligomers were added to this culture (FIG. 4M through FIG. 4O), indicating Aβ oligomers may enhance the function of Vangl2 in disrupting the Celsr3-Frizzled3 complex across the cell-cell junction.

Although Aβ oligomers are not sufficient to disrupt the complex between Celsr3 and Frizzled3 (FIG. 4J), it was found that Aβ oligomers can disrupt the intercellular complex, as the HA-tagged Frizzled3 in one cell pulled down less Flag-tagged Celsr3 from a neighboring cells when Aβ oligomers were added to the mixed culture (FIGS. 4P-4R). This can be because the intercellular interaction of Celsr3 may weaken the intracellular interaction between Celsr3 and Frizzled3 such that Aβ oligomers can now affect. In glutamatergic synapses, Celsr3 is present on both pre- and post-synaptic sides. So Aβ oligomers would be able to disrupt the intercellular complex by disrupting the intercellular Frizzled3-Celsr3 complex. This would be in the same direction as Vangl2.

Finally, it was found that adding Aβ oligomers to mixed cultures with Vangl2 expressed with Celsr3 lead to the greatest disruption of this intercellular complex (FIG. 4S through FIG. 4U). Therefore, it is proposed that Aβ oligomers enhance the function of Vangl2 by disrupting the Celsr3/Frizzled3 intracellular complex and thus the asymmetric Celsr3/Frizzled3-Celsr3 intercellular complex essential for PCP signaling. This may be because Aβ oligomers bind to the Laminin G1 domain of Celsr3, which mediates Celsr3/Frizzled3 complex formation, and weaken the interaction between Celsr3 and Frizzled3, allowing Vangl2 to more efficiently disrupt the asymmetric intercellular complex of Celsr3/Frizzled3-Celsr3 and thus disassemble more synapses (FIG. 4V).

### Example 3

### Aβ Oligomers Bind to Celsr3 and Disrupts the Same Intercellular Complexes

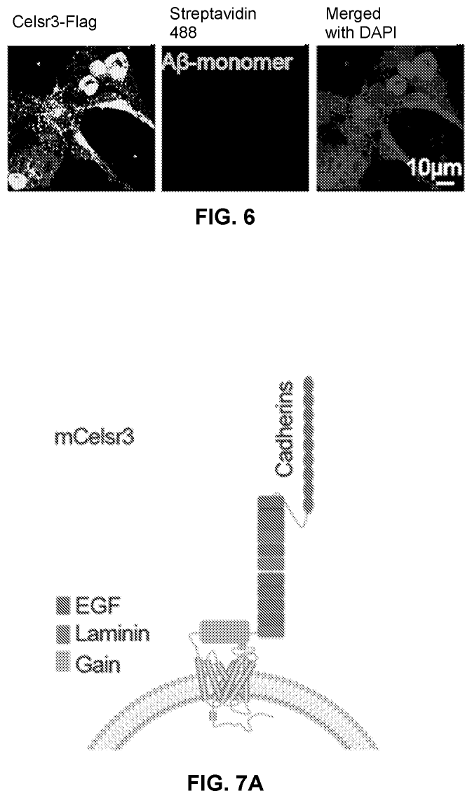

PCP components are distributed in glutamatergic synapses analogous to their organization in asymmetric epithelial cell junctions with membrane location of Frizzled3, Celsr3 and Vangl2. The following studies were performed to examine and demonstrate whether Aβ oligomers target any one(s) of those three proteins. Particularly, binding of biotin-Aβ42 oligomers to HEK293T cells that expressed Vangl2 (Vangl2-Flag), Frizzled3 (Frizzled3-HA), mouse Celsr3 (Celsr3-Flag) or control vector (pCAGEN) was measured. It was found that Aβ oligomers bound to Celsr3, but not Vangl2 or Frizzled3 (FIG. 5A), with an apparent dissociation constant (Kd) of ˜40 nM equivalent of total Aβ peptide (FIG. 5B). Aβ monomer could not bind to Celsr3 (FIG. 6).

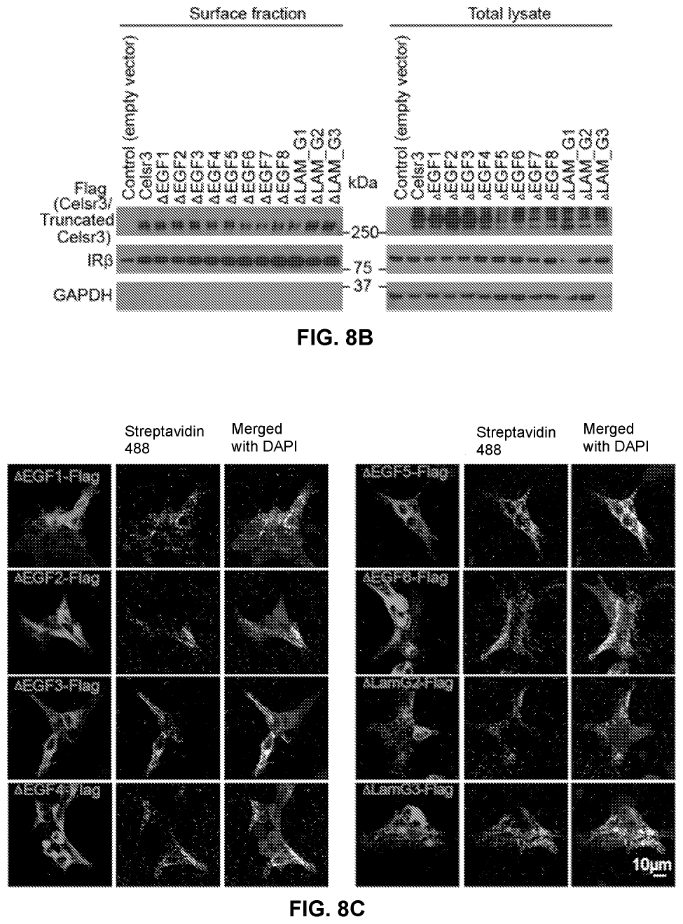

Celsr3 belongs to the family of adhesion G-protein coupled receptors with a large extracellular region, which contains 9 cadherin domains, 8 EGF repeats and 3 laminin domains (FIG. 7A). Cadherin domains are considered as homophilic binding regions. To determine the domains of Celsr3 responsible for binding with Aβ oligomers, deletion constructs were made. It was found that Aβ oligomers did not bind to the cadherin domains (FIG. 8A and FIG. 7B). Another series of Celsr3 constructs that lack these individual EGF and laminin domains were made (FIG. 8B) and found that two EGF domains, EGF7 and EGF8 and one Laminin domain, Laminin-G1 were required for binding of Aβ (FIG. 8C and FIG. 7B).

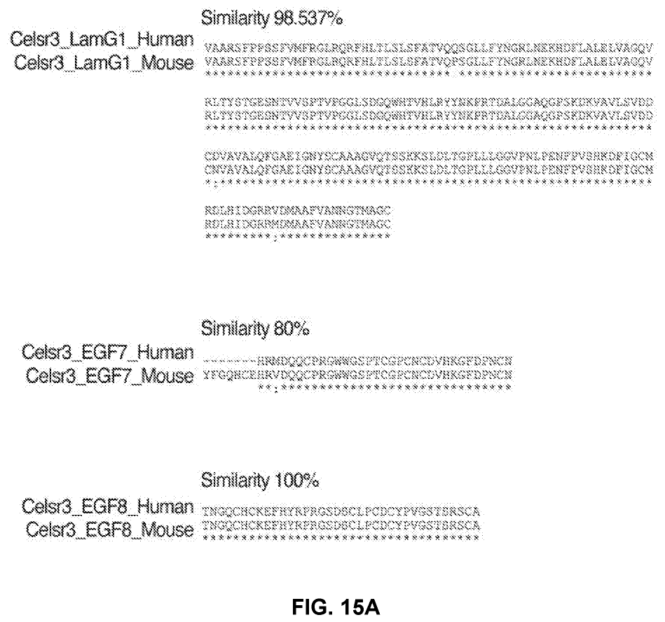

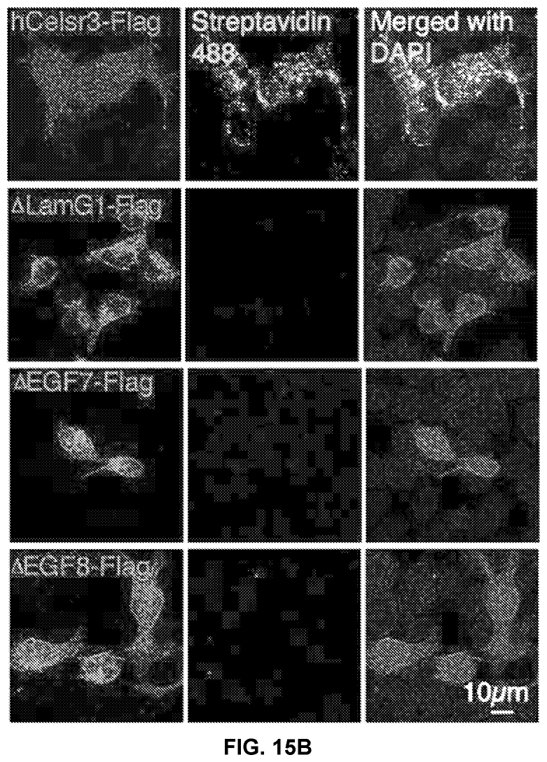

The human homolog of murine Celsr3 also contains 9 cadherin domains, 8 EGF repeats and 3 laminin domains. The Laminin G1 and EGF7 domains of hCelsr3 aligns closely with that of mCelsr3 with homology of 98.537% and 80%, respectively. The amino acid sequence of the EGF8 domain of hCelsr3 is 100% homologous with that of the EGF8 domain of mCelsr3 (FIG. 15A). It was found that Aβ oligomers also bound to hCelsr3. Like with the mCelsr3, EGF7 and EGF8 and one Laminin domain, Laminin-G1 of hCelsr3 are required for binding with Aβ oligomers (FIG. 15B).

The fact that Aβ oligomers only bind to Celsr3 but not Vangl2 suggests that Aβ oligomers can enhance the function of Vangl2 via affecting the interactions among PCP components. Frizzled3 or Vangl2 were expressed together with wild type Celsr3 or truncated Celsr3 in HEK293T cells. It was found that deletion of all 8 EGF repeats and 3 Laminin domains caused a 68% reduction of the interaction between Frizzled3 and Celsr3. Deletion of Laminin G1 leads to 66% reduction of the interaction between Frizzled3 and Celsr3 (FIG. 9A). The interaction between Vangl2 and Ceslr3 did not require EGF repeats and Laminin domains (FIG. 9B). Therefore, these data demonstrated that Aβ oligomers can enhance the function of Vangl2 in disrupting the Celsr3-Frizzled3 intracellular complex and/or Ceslr3/Frizzled3-Celsr3 intercellular complex by binding to the Laminin G1 domain of Celsr3, which mediates Celsr3/Frizzled3 complex formation.

### Example 4

### Vangl2 Conditional Knock-Out (cKO) Reduces Glutamatergic Synapse Loss in a Mouse Model of Alzheimer's Disease

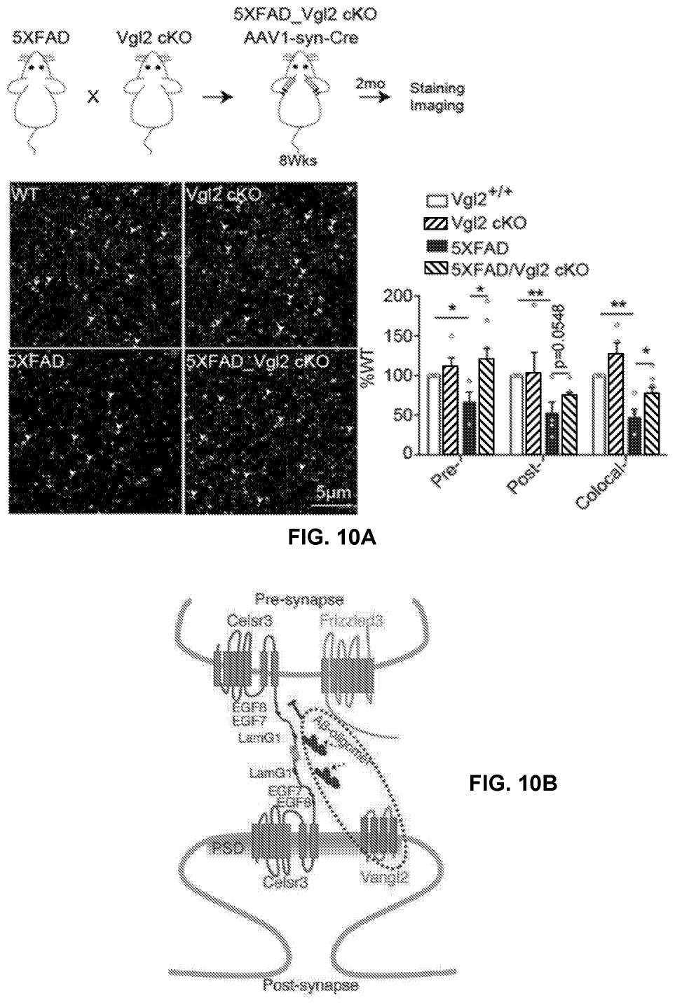

To characterize the overall effect of Vangl2 on synapse loss in Aβ transgenic mouse model, Vangl2 cKO mice were crossed with 5×FAD transgenic mice. AAV-Cre was injected into the hippocampal CA1 region of 8-week-old mice for 2 months. 5×FAD transgenic mice had significant reduction of synapse numbers. 5×FAD; Vangl2 cKO transgenic mice showed an improved synapse numbers (FIG. 10A).

The studies described in above Examples 1 to 4 identified Celsr3 as a receptor for Aβ oligomers and the PCP signaling as a direct target of Aβ oligomers for synaptotoxicity. Cell-cell interaction is important for the establishment and maintenance of cell and tissue polarity along the tissue plan. Celsr3 forms a complex with Frizzled3 on the plasma membrane of one cell and interact with a Celsr3/Vangl2 complex on the plasma membrane of the neighboring cells. These components then form an intercellular complex, using Celsr3 as a bridge. PCP components are localized similarly in glutamatergic synapses and regulates synapse formation. Frizzled3 is enriched on the presynaptic membrane and Vangl2 is only localized on the postsynaptic density (FIG. 10B) whereas Celsr3 is on both membranes. These studies show that Vangl2 disrupts the complex between Frizzled3 and Celsr3 in the neighboring cell as well as the intercellular complex. Aβ oligomers binds to three of the Celsr3 domains, one of which mediates the formation of Frizzled3/Celsr3 complex. And Aβ oligomers disrupt this intercellular complex. These data suggest that that Aβ oligomers bind to Celsr3 and weaken the PCP complex in the synapses, allowing Vangl2 to disrupt the PCP complex, leading to disassembly of glutamatergic synapses. Several receptors have been found to bind to Aβ oligomers regulating synaptic plasticity, such as cellular prion protein (PrP^{C}), EphB2 and paired immunoglobulin-like receptor B (PirB) or its human ortholog leukocyte immunoglobulin-like receptor B2 (LilrB2) and alters synaptic function and plasticity but not synapse loss. The above study is the first to identify the receptor and the signaling pathway that directly mediates synapse loss.

Preventing Aβ oligomer-induced synapse loss can at least slow down disease progression in the Alzheimer's disease patients with overproduction resulted from mutations in enzymes or the cellular processes that produce overproduction. Even in mutations of apolipoprotein E4, a major risk factor, Aβ peptides are over produced and preventing Aβ oligomers from targeting the PCP pathway may benefit these patients. The above studies and data suggest that blocking synapse loss by either preventing Aβ oligomer binding to Celsr3 or by inhibiting the function of Vangl2 can stop neuronal death or even restore disconnected neural circuits and memory. These findings provide new therapeutic targets for treatment of Alzheimer's disease.

### Example 5

### The Wnt/Vangl2/Ryk Signaling Axis Mediates Synapse Loss Induced by Oligomeric Aβ

Noncanonical Wnt signaling inhibits glutamatergic synapse formation via a PCP component Celsr3. Ryk is a co-receptor for Wnt in PCP signaling via interactions with Vangl2. The following study was performed to examine and demonstrate whether Ryk mediates Wnt5a signaling in synapse number regulation and does so in a Vangl2-dependent manner.

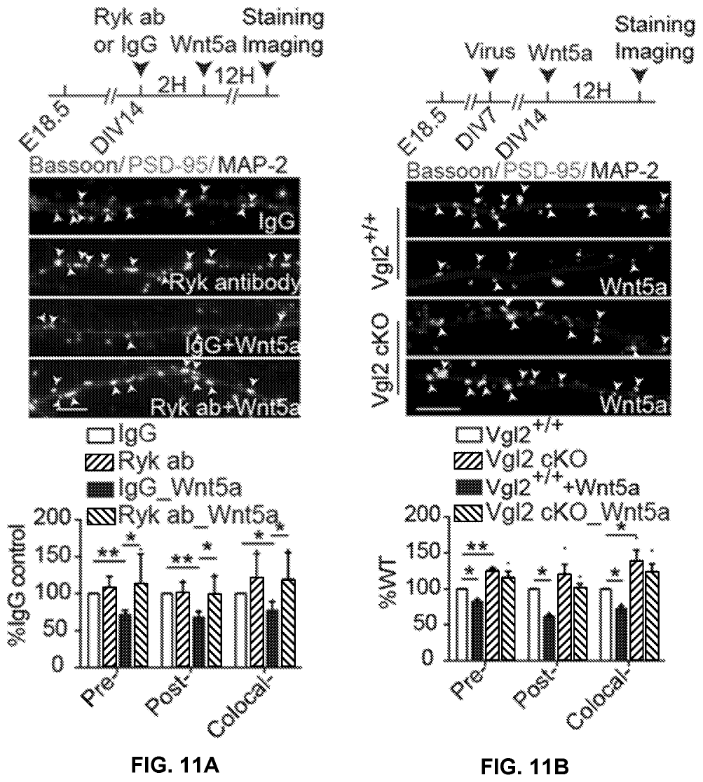

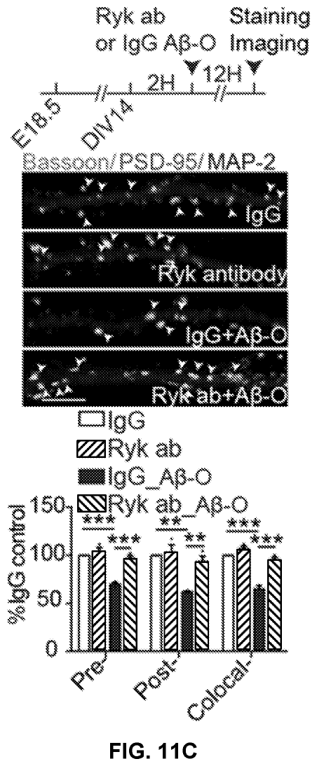

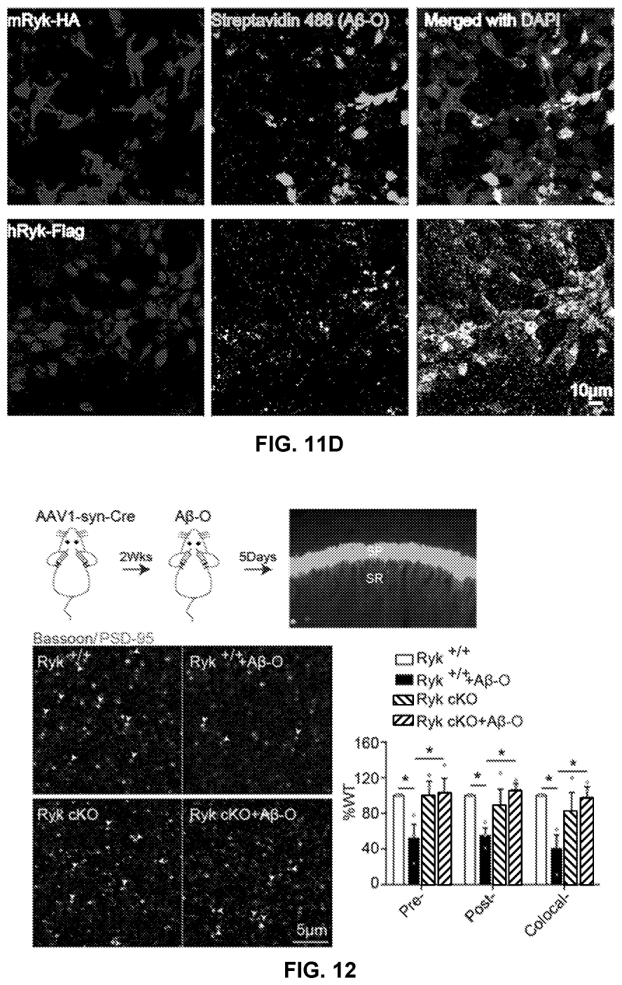

Hippocampal neurons isolated from E18.5 WT mice were treated with Wnt5a on DIV14 for 12 hours or pre-treated with a function blocking monoclonal Ryk antibody (blocks binding between Wnts and Ryk) for 2 hours (FIG. 11A). Mouse IgG was used as control. Wnt5a caused 30% reduction in the number of colocalized puncta. In contrast, Wnt5a did not produce a significant difference in synapse number when pre-treated with Ryk antibody for 2 h (FIG. 11A), suggesting that Wnt5a inhibits synapse formation through binding to Ryk as the receptor. There was no significant difference in synapse number in Ryk antibody treated WT neurons compared with IgG treated WT neurons.

To examine whether Vangl2 mediates the inhibitory function of Wnts in synapse formation downstream of Ryk, Vangl2^{+/+} and Vangl2 cKO embryonic hippocampal neurons (infected with AAV-Cre) were cultured and treated with Wnt5a on DIV 14 for 12 h. It was found that Wnt5a addition to Vangl2^{+/+} showed a 30% reduction in the number of colocalized puncta. Wnt5a addition to Vangl2 cKO neurons did not produce a significant difference compared with untreated Vangl2 cKO neurons (FIG. 11B), suggesting that Vangl2 is required for the inhibitory function of Wnt5a in synapse formation.

Then cultured hippocampal neurons were pre-treated with Ryk antibody 2 h before Aβ oligomers challenging and it was found that Aβ oligomers did not induce significant reduction of synapse number in the presence of Ryk antibody (FIG. 11C). Binding assay showed that Aβ oligomers did not bind to either mouse Ryk (mouse Ryk-HA) or human Ryk (human Ryk-Flag) expressed HEK 293T cells (FIG. 11D).

### Example 6

### Ryk is Required for Oligomeric Amyloid Beta-Mediated Synaptotoxicity In Vivo

The following studies were performed to examine and demonstrate whether the Wnt/Ryk signaling module is required for Aβ oligomer mediated synapse loss. Aβ oligomers were injected into Ryk cKO mice. In Ryk^{+/+}, Aβ oligomers induced a 60% reduction of synapse, but not in Aβ oligomers injected Ryk cKO mice (FIG. 12A).

### Example 7

### Ryk cKO Increases Synapse Number and Improves Cognitive Functions in a Mouse Model of Alzheimer's Disease

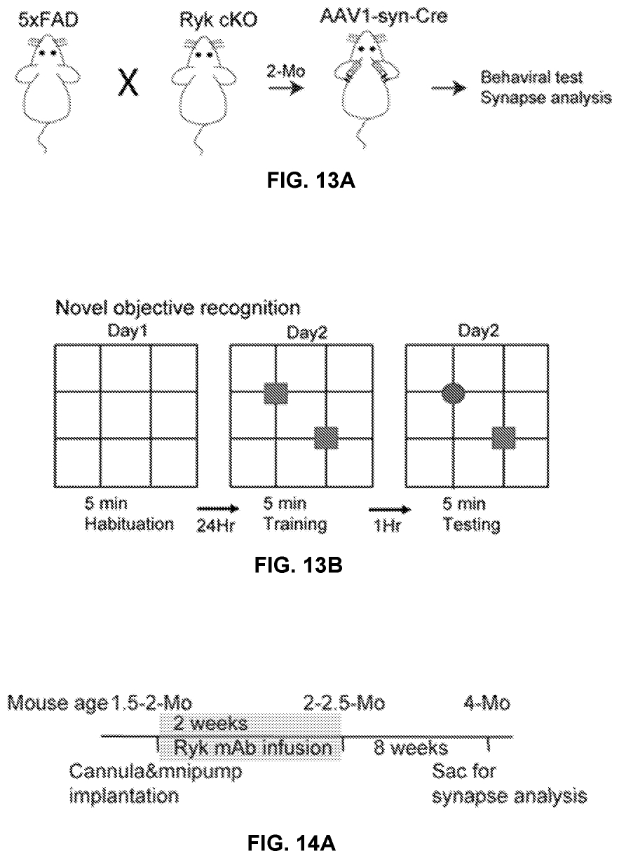

The following studies were performed to characterize the overall effect of Ryk on synapse loss in Aβ transgenic mouse model. Ryk cKO mice were crossed with 5×FAD transgenic mice. AAV-Cre was injected into the hippocampal CA1 region of 8-week-old mice for 2 months (FIG. 13A).

The mice are subjected to the procedure as shown in FIG. 13B for examining objective recognition. The mice are then scarified, and tissues are harvested from the mice and analyzed by microscopes. The results show that Ryk cKO increases synapse number and improves cognitive functions in the mouse model of Alzheimer's disease.

### Example 8

### Ryk is a Novel Therapeutic Target for Alzheimer's Disease

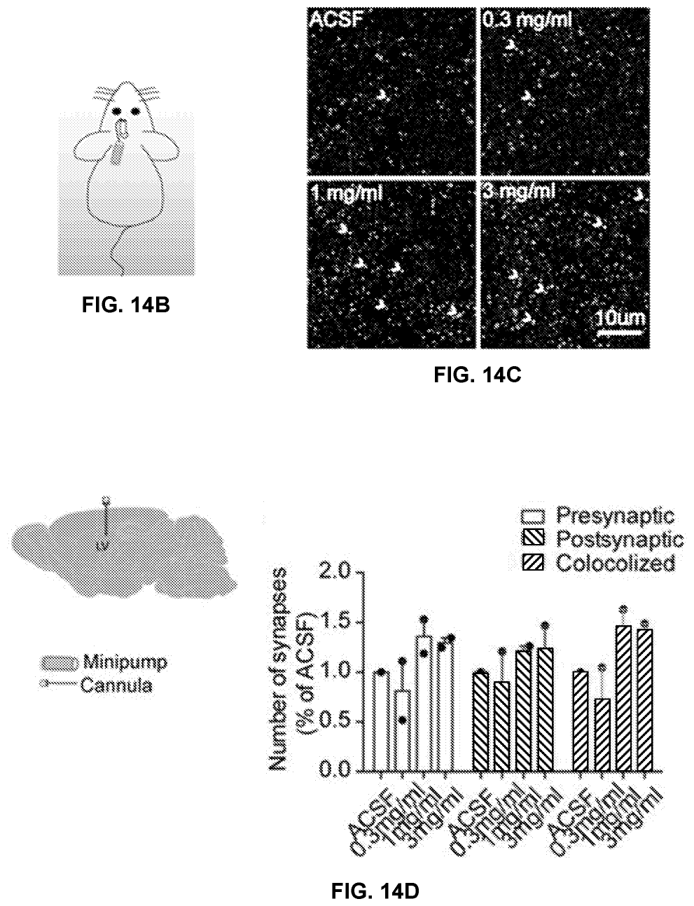

The following studies were performed to examine and demonstrate whether inhibition of the Wnt-Ryk signaling axis in transgenic mouse is sufficient to Aβ-related pathology recovery. In order to test whether the Ryk monoclonal antibody can be used as a therapeutic agent to block amyloid-beta induced synapse loss in Alzheimer's disease, the monoclonal Ryk antibody was intracerebrally infused for 2 weeks into transgenic Aβ mouse (FIG. 14A and FIG. 14B). The result showed that that the synapse numbers are rescued by the infusion of the monoclonal Ryk antibody.

The studies described in above Examples 5 to 8 demonstrated that both Ryk and Vangl2 are required for Wnt5a mediated inhibition of synapse formation. An anti-Ryk antibody, which blocks Wnt-Ryk binding, can block Aβ oligomer-mediated synapse loss. These results suggest that Vangl2 requires the active participation of Ryk, which is activated by Wnt5a, to remove synapses. Finally, these studies provided genetic evidence that Vangl2 and Ryk are both required for Aβ oligomer-mediated synapse loss in vitro and in vivo.

1. A method for reducing or preventing amyloid beta (Aβ) induced loss of synapses in a population of neurons, comprising contacting the neurons with an effective amount of an Aβ inhibitor that blocks binding of Aβ to Celsr,
and optionally wherein the method further comprises contacting the population of neurons with one or more of: a Ryk inhibitor, a Vangl inhibitor, a Celsr agonist, or a Frizzled agonist.

2. The method of claim 1, wherein the Aβ inhibitor competes with Aβ for binding to the EGF7, EGF8, and/or Laminin G1 domains of Celsr.

3. The method of claim 2, wherein the Aβ inhibitor competes with Aβ for binding to the Laminin G1 domain of Celsr.

4. The method of claim 1, wherein the Aβ is oligomeric Aβ.

5. The method of claim 1, wherein the Aβ inhibitor is an anti-Celsr antibody or antigen binding fragment thereof.

6. (canceled)

7. The method of claim 5, wherein the anti-Celsr antibody specifically binds to Celsr3.

8. The method of claim 4, wherein the anti-Celsr antibody preferentially binds to Celsr3 over Celsr2.

9. The method of claim 1, wherein the Aβ inhibitor competes with Celsr3 for binding with Aβ.

10. The method of claim 9, wherein the Aβ inhibitor comprises a Celsr3 peptide.

11. The method of claim 10, wherein the Celsr3 peptide comprises: (a) one or more Laminin G1 domain of Celsr or a functional variant thereof, (b) one or more EGF7 domain of Celsr or a functional variant thereof, (c) one or more EGF8 domain of Celsr or a functional variant thereof, (d) one or more extracellular domain of Celsr or a function variant thereof, or (e) any combination of (a) to (d).

12. The method of claim 9, wherein the Aβ inhibitor comprises a Celsr 3 peptide fused to an immunoglobulin Fc region.

13-18. (canceled)

19. The method of claim 9, wherein the Aβ inhibitor is an antibody or antigen binding fragment that binds to Aβ.

20.-23. (canceled)

24. A method of modulating formation of synapses in a population of neurons, comprising modulating one or more planar cell polarity (PCP) signaling pathway component and/or one or more non-canonical Wnt signaling pathway component.

25. A method of modulating maintenance of synapses in a population of neurons, comprising modulating one or more planar cell polarity (PCP) signaling pathway component and/or one or more non-canonical Wnt signaling pathway component.

26.-42. (canceled)

43. A method of managing, preventing, or treating a neurodegenerative disease in a subject, comprising administering to the subject a therapeutically effective amount of an amyloid beta (Aβ) inhibitor that blocks binding of Aβ to Celsr.

44. The method of claim 43, wherein the Aβ inhibitor competes with Aβ for binding to EGF7, EGF8, and/or Laminin G1 domains of Celsr.

45. The method of claim 43, wherein the Aβ is oligomeric Aβ.

46.-49. (canceled)

50. The method of claim 43, wherein the Aβ inhibitor competes with Celsr3 for binding with Aβ.

51. The method of claim 50, wherein the Aβ inhibitor comprises a Celsr3 peptide.

52. The method of claim 51, wherein the Celsr3 peptide comprises: (a) one or more Laminin G1 domain of Celsr or a functional variant thereof, (b) one or more EGF7 domain of Celsr or a functional variant thereof, (c) one or more EGF8 domain of Celsr or a functional variant thereof, (d) one or more extracellular domain of Celsr or a function variant thereof, or (e) any combination of (a) to (d).

53. The method of claim 50, wherein the Aβ inhibitor comprises a Celsr3 peptide fused to an immunoglobulin Fc region.

54.-59. (canceled)

60. The method of claim 50, wherein the Aβ inhibitor is an antibody or antigen binding fragment that binds to Aβ.

61.-117. (canceled)

118. A method of selecting an amyloid beta (Aβ) inhibitor that prevents or reduces Aβ-mediated neurotoxicity, comprising:
(a) contacting a candidate agent with Celsr or a Celsr variant in the presence of Aβ; and
(b) selecting the candidate agent as the Aβ inhibitor if the candidate agent reduces or inhibits binding of Aβ to the Celsr or Celsr variant.

119. The method of claim 118, wherein the Celsr or Celsr variant is expressed on the surface of a cell.

120. The method of claim 119, wherein the cell is a neuron.

121. The method of claim 119, wherein the cell is in an in vitro cell culture.

122. The method of claim 119, wherein the cell is in a non-human mammal cell.

123. The method of claim 118, wherein the Celsr or Celsr variant is immobilized on a solid support.

124.-139. (canceled)


SEQUENCE LISTING (41 sequences)

SEQ ID NO: 1 - DNA, 30 bp, Artificial Sequence
  Synthetic construct
gatcgatatcttctctggagagctcacagc

SEQ ID NO: 2 - DNA, 30 bp, Artificial Sequence
  Synthetic construct
gcaggcatcgtaaaagggcagcacgtcgag

SEQ ID NO: 3 - DNA, 30 bp, Artificial Sequence
  Synthetic construct
gtgctgcccttttacgatgcctgccccaag

SEQ ID NO: 4 - DNA, 25 bp, Artificial Sequence
  Synthetic construct
gatcgctagcaagtaggccagcaag

SEQ ID NO: 5 - DNA, 32 bp, Artificial Sequence
  Synthetic construct
tgctgccctttacagagctcgacctctgttac

SEQ ID NO: 6 - DNA, 28 bp, Artificial Sequence
  Synthetic construct
cgagctctgtaaagggcagcacgtcgag

SEQ ID NO: 7 - DNA, 31 bp, Artificial Sequence
  Synthetic construct
tctgtgagacactggacactgaagctggacg

SEQ ID NO: 8 - DNA, 32 bp, Artificial Sequence
  Synthetic construct
tcagtgtccagtgtctcacaagaagtctcccg

SEQ ID NO: 9 - DNA, 29 bp, Artificial Sequence
  Synthetic construct
gctggacactgtggccgcacgctcctttc

SEQ ID NO: 10 - DNA, 28 bp, Artificial Sequence
  Synthetic construct
gtgcggccacagtgtccagctcgcagtc

SEQ ID NO: 11 - DNA, 32 bp, Artificial Sequence
  Synthetic construct
acgctgtgagcaggccaagtcacacttttgtg

SEQ ID NO: 12 - DNA, 28 bp, Artificial Sequence
  Synthetic construct
acttggcctgctcacagcgtggaccatc

SEQ ID NO: 13 - DNA, 31 bp, Artificial Sequence
  Synthetic construct
aggctgccagctcacaatggcccatccctac

SEQ ID NO: 14 - DNA, 29 bp, Artificial Sequence
  Synthetic construct
ccattgtgagctggcagcctgccatagtg

SEQ ID NO: 15 - DNA, 29 bp, Artificial Sequence
  Synthetic construct
ctgtcgactcactgtgaccaacccctgtg

SEQ ID NO: 16 - DNA, 31 bp, Artificial Sequence
  Synthetic construct
tggtcacagtgagtcgacagtctttgccacc

SEQ ID NO: 17 - DNA, 29 bp, Artificial Sequence
  Synthetic construct
tggctgtactgatgcctgcctcctgaacc

SEQ ID NO: 18 - DNA, 32 bp, Artificial Sequence
  Synthetic construct
ggcaggcatcagtacagccaggctccacattc

SEQ ID NO: 19 - DNA, 34 bp, Artificial Sequence
  Synthetic construct
aggctgtgtgtattttggtcagcactgtgagcac

SEQ ID NO: 20 - DNA, 32 bp, Artificial Sequence
  Synthetic construct
gctgaccaaaatacacacagcctgggccatag

SEQ ID NO: 21 - DNA, 29 bp, Artificial Sequence
  Synthetic construct
tgtgagtggcaagacgaatggccagtgcc

SEQ ID NO: 22 - DNA, 29 bp, Artificial Sequence
  Synthetic construct
ccattcgtcttgccactcacagtcacaag

SEQ ID NO: 23 - DNA, 27 bp, Artificial Sequence
  Synthetic construct
caactgcaacccccacagcgggcagtg

SEQ ID NO: 24 - DNA, 28 bp, Artificial Sequence
  Synthetic construct
ctgtgggggttgcagttggggtcaaagc

SEQ ID NO: 25 - DNA, 30 bp, Artificial Sequence
  Synthetic construct
gcatcgtagagtgggaggcatgagtcactg

SEQ ID NO: 26 - DNA, 31 bp, Artificial Sequence
  Synthetic construct
atgccacccactctacgatgcctgccccaag

SEQ ID NO: 27 - protein, 42 aa, Homo sapiens [sequence omitted]

SEQ ID NO: 28 - protein, 3014 aa, Homo sapiens
MAPPPPPVLPVLLLLAAAAALPAMGLRAAAWEPRVPGGTRAFALRPGCTYAVGAACTPRAPRELLDVGRDGRLAGRRRVSGAGRPLPLQVRLVARSAPTALSRRLRARTHLPGCGARARLCGTGARLCGALCFPVPGGCAAAQHSALAAPTTLPACRCPPRPRPRCPGRPICLPPGGSVRLRLLCALRRAAGAVRVGLALEAATAGTPSASPSPSPPLPPNLPEARAGPARRARRGTSGRGSLKFPMPNYQVALFENEPAGTLILQLHAHYTIEGEEERVSYYMEGLFDERSRGYFRIDSATGAVSTDSVLDRETKETHVLRVKAVDYSTPPRSATTYITVLVKDTNDHSPVFEQSEYRERVRENLEVGYEVLTIRASDRDSPINANLRYRVLGGAWDVFQLNESSGVVSTRAVLDREEAAEYQLLVEANDQGRNPGPLSATATVYIEVEDENDNYPQFSEQNYVVQVPEDVGLNTAVLRVQATDRDQGQNAAIHYSILSGNVAGQFYLHSLSGILDVINPLDFEDVQKYSLSIKAQDGGRPPLINSSGVVSVQVLDVNDNEPIFVSSPFQATVLENVPLGYPVVHIQAVDADSGENARLHYRLVDTASTFLGGGSAGPKNPAPTPDFPFQIHNSSGWITVCAELDREEVEHYSFGVEAVDHGSPPMSSSTSVSITVLDVNDNDPVFTQPTYELRLNEDAAVGSSVLTLQARDRDANSVITYQLTGGNTRNRFALSSQRGGGLITLALPLDYKQEQQYVLAVTASDGTRSHTAHVLINVTDANTHRPVFQSSHYTVSVSEDRPVGTSIATLSANDEDTGENARITYVIQDPVPQFRIDPDSGTMYTMMELDYENQVAYTLTIMAQDNGIPQKSDTTTLEILILDANDNAPQFLWDFYQGSIFEDAPPSTSILQVSATDRDSGPNGRLLYTFQGGDDGDGDFYIEPTSGVIRTQRRLDRENVAVYNLWALAVDRGSPTPLSASVEIQVTILDINDNAPMFEKDELELFVEENNPVGSVVAKIRANDPDEGPNAQIMYQIVEGDMRHFFQLDLLNGDLRAMVELDFEVRREYVLVVQATSAPLVSRATVHILLVDQNDNPPVLPDFQILFNNYVTNKSNSFPTGVIGCIPAHDPDVSDSLNYTFVQGNELRLLLLDPATGELQLSRDLDNNRPLEALMEVSVSDGIHSVTAFCTLRVTIITDDMLTNSITVRLENMSQEKFLSPLLALFVEGVAAVLSTTKDDVFVFNVQNDTDVSSNILNVTFSALLPGGVRGQFFPSEDLQEQIYLNRTLLTTISTQRVLPFDDNICLREPCENYMKCVSVLRFDSSAPFLSSTTVLFRPIHPINGLRCRCPPGFTGDYCETEIDLCYSDPCGANGRCRSREGGYTCECFEDFTGEHCEVDARSGRCANGVCKNGGTCVNLLIGGFHCVCPPGEYERPYCEVTTRSFPPQSFVTFRGLRQRFHFTISLTFATQERNGLLLYNGRFNEKHDFIALEIVDEQVQLTFSAGETTTTVAPKVPSGVSDGRWHSVQVQYYNKPNIGHLGLPHGPSGEKMAVVTVDDCDTTMAVRFGKDIGNYSCAAQGTQTGSKKSLDLTGPLLLGGVPNLPEDFPVHNRQFVGCMRNLSVDGKNVDMAGFIANNGTREGCAARRNFCDGRRCQNGGTCVNRWNMYLCECPLRFGGKNCEQAMPHPQLFSGESVVSWSDLNIIISVPWYLGLMFRTRKEDSVLMEATSGGPTSFRLQILNNYLQFEVSHGPSDVESVMLSGLRVTDGEWHHLLIELKNVKEDSEMKHLVTMTLDYGMDQNKADIGGMLPGLTVRSVVVGGASEDKVSVRRGFRGCMQGVRMGGTPTNVATLNMNNALKVRVKDGCDVDDPCTSSPCPPNSRCHDAWEDYSCVCDKGYLGINCVDACHLNPCENMGACVRSPGSPQGYVCECGPSHYGPYCENKLDLPCPRGWWGNPVCGPCHCAVSKGFDPDCNKTNGQCQCKENYYKLLAQDTCLPCDCFPHGSHSRTCDMATGQCACKPGVIGRQCNRCDNPFAEVTTLGCEVIYNGCPKAFEAGIWWPQTKFGQPAAVPCPKGSVGNAVRHCSGEKGWLPPELFNCTTISFVDLRAMNEKLSRNETQVDGARALQLVRALRSATQHTGTLFGNDVRTAYQLLGHVLQHESWQQGFDLAATQDADFHEDVIHSGSALLAPATRAAWEQIQRSEGGTAQLLRRLEGYFSNVARNVRRTYLRPFVIVTANMILAVDIFDKFNFTGARVPRFDTIHEEFPRELESSVSFPADFFRPPEEKEGPLLRPAGRRTTPQTTRPGPGTEREAPISRRRRHPDDAGQFAVALVIIYRTLGQLLPERYDPDRRSLRLPHRPIINTPMVSTLVYSEGAPLPRPLERPVLVEFALLEVEERTKPVCVFWNHSLAVGGTGGWSARGCELLSRNRTHVACQCSHTASFAVLMDISRRENGEVLPLKIVTYAAVSLSLAALLVAFVLLSLVRMLRSNLHSIHKHLAVALFLSQLVFVIGINQTENPFLCTVVAILLHYIYMSTFAWTLVESLHVYRMLTEVRNIDTGPMRFYYVVGWGIPAIVTGLAVGLDPQGYGNPDFCWLSLQDTLIWSFAGPIGAVIIINTVTSVLSAKVSCQRKHHYYGKKGIVSLLRTAFLLLLLISATWLLGLLAVNRDALSFHYLFAIFSGLQGPFVLLFHCVLNQEVRKHLKGVLGGRKLHLEDSATTRATLLTRSLNCNTTFGDGPDMLRTDLGESTASLDSIVRDEGIQKLGVSSGLVRGSHGEPDASLMPRSCKDPPGHDSDSDSELSLDEQSSSYASSHSSDSEDDGVGAEEKWDPARGAVHSTPKGDAVANHVPAGWPDQSLAESDSEDPSGKPRLKVETKVSVELHREEQGSHRGEYPPDQESGGAARLASSQPPEQRKGILKNKVTYPPPLTLTEQTLKGRLREKLADCEQSPTSSRTSSLGSGGPDCAITVKSPGREPGRDHLNGVAMNVRTGSAQADGSDSEKP

SEQ ID NO: 29 - protein, 2923 aa, Homo sapiens
MRSPATGVPLPTPPPPLLLLLLLLLPPPLLGDQVGPCRSLGSRGRGSSGACAPMGWLCPSSASNLWLYTSRCRDAGTELTGHLVPHHDGLRVWCPESEAHIPLPPAPEGCPWSCRLLGIGGHLSPQGKLTLPEEHPCLKAPRLRCQSCKLAQAPGLRAGERSPEESLGGRRKRNVNTAPQFQPPSYQATVPENQPAGTPVASLRAIDPDEGEAGRLEYTMDALFDSRSNQFFSLDPVTGAVTTAEELDRETKSTHVFRVTAQDHGMPRRSALATLTILVTDTNDHDPVFEQQEYKESLRENLEVGYEVLTVRATDGDAPPNANILYRLLEGSGGSPSEVFEIDPRSGVIRTRGPVDREEVESYQLTVEASDQGRDPGPRSTTAAVFLSVEDDNDNAPQFSEKRYVVQVREDVTPGAPVLRVTASDRDKGSNAVVHYSIMSGNARGQFYLDAQTGALDVVSPLDYETTKEYTLRVRAQDGGRPPLSNVSGLVTVQVLDINDNAPIFVSTPFQATVLESVPLGYLVLHVQAIDADAGDNARLEYRLAGVGHDFPFTINNGTGWISVAAELDREEVDFYSFGVEARDHGTPALTASASVSVTVLDVNDNNPTFTQPEYTVRLNEDAAVGTSVVTVSAVDRDAHSVITYQITSGNTRNRFSITSQSGGGLVSLALPLDYKLERQYVLAVTASDGTRQDTAQIVVNVTDANTHRPVFQSSHYTVNVNEDRPAGTTVVLISATDEDTGENARITYFMEDSIPQFRIDADTGAVTTQAELDYEDQVSYTLAITARDNGIPQKSDTTYLEILVNDVNDNAPQFLRDSYQGSVYEDVPPFTSVLQISATDRDSGLNGRVFYTFQGGDDGDGDFIVESTSGIVRTLRRLDRENVAQYVLRAYAVDKGMPPARTPMEVTVTVLDVNDNPPVFEQDEFDVFVEENSPIGLAVARVTATDPDEGTNAQIMYQIVEGNIPEVFQLDIFSGELTALVDLDYEDRPEYVLVIQATSAPLVSRATVHVRLLDRNDNPPVLGNFEILFNNYVTNRSSSFPGGAIGRVPAHDPDISDSLTYSFERGNELSLVLLNASTGELKLSRALDNNRPLEAIMSVLVSDGVHSVTAQCALRVTIITDEMLTHSITLRLEDMSPERFLSPLLGLFIQAVAATLATPPDHVVVFNVQRDTDAPGGHILNVSLSVGQPPGPGGGPPFLPSEDLQERLYLNRSLLTAISAQRVLPFDDNICLREPCENYMRCVSVLRFDSSAPFIASSSVLFRPIHPVGGLRCRCPPGFTGDYCETEVDLCYSRPCGPHGRCRSREGGYTCLCRDGYTGEHCEVSARSGRCTPGVCKNGGTCVNLLVGGFKCDCPSGDFEKPYCQVTTRSFPAHSFITFRGLRQRFHFTLALSFATKERDGLLLYNGRFNEKHDFVALEVIQEQVQLTFSAGESTTTVSPFVPGGVSDGQWHTVQLKYYNKPLLGQTGLPQGPSEQKVAVVTVDGCDTGVALRFGSVLGNYSCAAQGTQGGSKKSLDLTGPLLLGGVPDLPESFPVRMRQFVGCMRNLQVDSRHIDMADFIANNGTVPGCPAKKNVCDSNTCHNGGTCVNQWDAFSCECPLGFGGKSCAQEMANPQHFLGSSLVAWHGLSLPISQPWYLSLMFRTRQADGVLLQAITRGRSTITLQLREGHVMLSVEGTGLQASSLRLEPGRANDGDWHHAQLALGASGGPGHAILSFDYGQQRAEGNLGPRLHGLHLSNITVGGIPGPAGGVARGFRGCLQGVRVSDTPEGVNSLDPSHGESINVEQGCSLPDPCDSNPCPANSYCSNDWDSYSCSCDPGYYGDNCTNVCDLNPCEHQSVCTRKPSAPHGYTCECPPNYLGPYCETRIDQPCPRGWWGHPTCGPCNCDVSKGFDPDCNKTSGECHCKENHYRPPGSPTCLLCDCYPTGSLSRVCDPEDGQCPCKPGVIGRQCDRCDNPFAEVTTNGCEVNYDSCPRAIEAGIWWPRTRFGLPAAAPCPKGSFGTAVRHCDEHRGWLPPNLFNCTSITFSELKGFAERLQRNESGLDSGRSQQLALLLRNATQHTAGYFGSDVKVAYQLATRLLAHESTQRGFGLSATQDVHFTENLLRVGSALLDTANKRHWELIQQTEGGTAWLLQHYEAYASALAQNMRHTYLSPFTIVTPNIVISVVRLDKGNFAGAKLPRYEALRGEQPPDLETTVILPESVFRETPPVVRPAGPGEAQEPEELARRQRRHPELSQGEAVASVIIYRTLAGLLPHNYDPDKRSLRVPKRPIINTPVVSISVHDDEELLPRALDKPVTVQFRLLETEERTKPICVFWNHSILVSGTGGWSARGCEVVFRNESHVSCQCNHMTSFAVLMDVSRRENGEILPLKTLTYVALGVTLAALLLTFFFLTLLRILRSNQHGIRRNLTAALGLAQLVFLLGINQADLPFACTVIAILLHFLYLCTFSWALLEALHLYRALTEVRDVNTGPMRFYYMLGWGVPAFITGLAVGLDPEGYGNPDFCWLSIYDTLIWSFAGPVAFAVSMSVFLYILAARASCAAQRQGFEKKGPVSGLQPSFAVLLLLSATWLLALLSVNSDTLLFHYLFATCNCIQGPFIFLSYVVLSKEVRKALKLACSRKPSPDPALTTKSTLTSSYNCPSPYADGRLYQPYGDSAGSLHSTSRSGKSQPSYIPFLLREESALNPGQGPPGLGDPGSLFLEGQDQQHDPDTDSDSDLSLEDDQSGSYASTHSSDSEEEEEEEEEEAAFPGEQGWDSLLGPGAERLPLHSTPKDGGPGPGKAPWPGDFGTTAKESSGNGAPEERLRENGDALSREGSLGPLPGSSAQPHKGILKKKCLPTISEKSSLLRLPLEQCTGSSRGSSASEGSRGGPPPRPPPRQSLQEQLNGVMPIAMSIKAGTVDEDSSGSEFLFFNFLH

SEQ ID NO: 30 - protein, 3312 aa, Homo sapiens
MMARRPPWRGLGGRSTPILLLLLLSLFPLSQEELGGGGHQGWDPGLAATTGPRAHIGGGALALCPESSGVREDGGPGLGVREPIFVGLRGRRQSARNSRGPPEQPNEELGIEHGVQPLGSRERETGQGPGSVLYWRPEVSSCGRTGPLQRGSLSPGALSSGVPGSGNSSPLPSDFLIRHHGPKPVSSQRNAGTGSRKRVGTARCCGELWATGSKGQGERATTSGAERTAPRRNCLPGASGSGPELDSAPRTARTAPASGSAPRESRTAPEPAPKRMRSRGLFRCRFLPQRPGPRPPGLPARPEARKVTSANRARFRRAANRHPQFPQYNYQTLVPENEAAGTAVLRVVAQDPDAGEAGRLVYSLAALMNSRSLELFSIDPQSGLIRTAAALDRESMERHYLRVTAQDHGSPRLSATTMVAVTVADRNDHSPVFEQAQYRETLRENVEEGYPILQLRATDGDAPPNANLRYRFVGPPAARAAAAAAFEIDPRSGLISTSGRVDREHMESYELVVEASDQGQEPGPRSATVRVHITVLDENDNAPQFSEKRYVAQVREDVRPHTVVLRVTATDRDKDANGLVHYNIISGNSRGHFAIDSLTGEIQVVAPLDFEAEREYALRIRAQDAGRPPLSNNTGLASIQVVDINDHIPIFVSTPFQVSVLENAPLGHSVIHIQAVDADHGENARLEYSLTGVAPDTPFVINSATGWVSVSGPLDRESVEHYFFGVEARDHGSPPLSASASVTVTVLDVNDNRPEFTMKEYHLRLNEDAAVGTSVVSVTAVDRDANSAISYQITGGNTRNRFAISTQGGVGLVTLALPLDYKQERYFKLVLTASDRALHDHCYVHINITDANTHRPVFQSAHYSVSVNEDRPMGSTIVVISASDDDVGENARITYLLEDNLPQFRIDADSGAITLQAPLDYEDQVTYTLAITARDNGIPQKADTTYVEVMVNDVNDNAPQFVASHYTGLVSEDAPPFTSVLQISATDRDAHANGRVQYTFQNGEDGDGDFTIEPTSGIVRTVRRLDREAVSVYELTAYAVDRGVPPLRTPVSIQVMVQDVNDNAPVFPAEEFEVRVKENSIVGSVVAQITAVDPDEGPNAHIMYQIVEGNIPELFQMDIFSGELTALIDLDYEARQEYVIVVQATSAPLVSRATVHVRLVDQNDNSPVLNNFQILFNNYVSNRSDTFPSGIIGRIPAYDPDVSDHLFYSFERGNELQLLVVNQTSGELRLSRKLDNNRPLVASMLVTVTDGLHSVTAQCVLRVVIITEELLANSLTVRLENMWQERFLSPLLGRFLEGVAAVLATPAEDVFIFNIQNDTDVGGTVLNVSFSALAPRGAGAGAAGPWFSSEELQEQLYVRRAALAARSLLDVLPFDDNVCLREPCENYMKCVSVLRFDSSAPFLASASTLFRPIQPIAGLRCRCPPGFTGDFCETELDLCYSNPCRNGGACARREGGYTCVCRPRFTGEDCELDTEAGRCVPGVCRNGGTCTDAPNGGFRCQCPAGGAFEGPRCEVAARSFPPSSFVMFRGLRQRFHLTLSLSFATVQQSGLLFYNGRLNEKHDFLALELVAGQVRLTYSTGESNTVVSPTVPGGLSDGQWHTVHLRYYNKPRTDALGGAQGPSKDKVAVLSVDDCDVAVALQFGAEIGNYSCAAAGVQTSSKKSLDLTGPLLLGGVPNLPENFPVSHKDFIGCMRDLHIDGRRVDMAAFVANNGTMAGCQAKLHFCDSGPCKNSGFCSERWGSFSCDCPVGFGGKDCQLTMAHPHHFRGNGTLSWNFGSDMAVSVPWYLGLAFRTRATQGVLMQVQAGPHSTLLCQLDRGLLSVTVTRGSGRASHLLLDQVTVSDGRWHDLRLELQEEPGGRRGHHVLMVSLDFSLFQDTMAVGSELQGLKVKQLHVGGLPPGSAEEAPQGLVGCIQGVWLGSTPSGSPALLPPSHRVNAEPGCVVTNACASGPCPPHADCRDLWQTFSCTCQPGYYGPGCVDACLLNPCQNQGSCRHLPGAPHGYTCDCVGGYFGHHCEHRMDQQCPRGWWGSPTCGPCNCDVHKGFDPNCNKTNGQCHCKEFHYRPRGSDSCLPCDCYPVGSTSRSCAPHSGQCPCRPGALGRQCNSCDSPFAEVTASGCRVLYDACPKSLRSGVWWPQTKFGVLATVPCPRGALGAAVRLCDEAQGWLEPDLFNCTSPAFRELSLLLDGLELNKTALDTMEAKKLAQRLREVTGHTDHYFSQDVRVTARLLAHLLAFESHQQGFGLTATQDAHFNENLLWAGSALLAPETGDLWAALGQRAPGGSPGSAGLVRHLEEYAATLARNMELTYLNPMGLVTPNIMLSIDRMEHPSSPRGARRYPRYHSNLFRGQDAWDPHTHVLLPSQSPRPSPSEVLPTSSSIENSTTSSVVPPPAPPEPEPGISIIILLVYRTLGGLLPAQFQAERRGARLPQNPVMNSPVVSVAVFHGRNFLRGILESPISLEFRLLQTANRSKAICVQWDPPGLAEQHGVWTARDCELVHRNGSHARCRCSRTGTFGVLMDASPRERLEGDLELLAVFTHVVVAVSVAALVLTAAILLSLRSLKSNVRGIHANVAAALGVAELLFLLGIHRTHNQLVCTAVAILLHYFFLSTFAWLFVQGLHLYRMQVEPRNVDRGAMRFYHALGWGVPAVLLGLAVGLDPEGYGNPDFCWISVHEPLIWSFAGPVVLVIVMNGTMFLLAARTSCSTGQREAKKTSALTLRSSFLLLLLVSASWLFGLLAVNHSILAFHYLHAGLCGLQGLAVLLLFCVLNADARAAWMPACLGRKAAPEEARPAPGLGPGAYNNTALFEESGLIRITLGASTVSSVSSARSGRTQDQDSQRGRSYLRDNVLVRHGSAADHTDHSLQAHAGPTDLDVAMFHRDAGADSDSDSDLSLEEERSLSIPSSESEDNGRTRGRFQRPLCRAAQSERLLTHPKDVDGNDLLSYWPALGECEAAPCALQTWGSERRLGLDTSKDAANNNQPDPALTSGDETSLGRAQRQRKGILKNRLQYPLVPQTRGAPELSWCRAATLGHRAVPAASYGRIYAGGGTGSLSQPASRYSSREQLDLLLRRQLSRERLEEAPAPVLRPLSRPGSQECMDAAPGRLEPKDRGSTLPRRQPPRDYPGAMAGRFGSRDALDLGAPREWLSTLPPPRRTRDLDPQPPPLPLSPQRQLSRDPLLPSRPLDSLSRSSNSREQLDQVPSRHPSREALGPLPQLLRAREDSVSGPSHGPSTEQLDILSSILASFNSSALSSVQSSSTPLGPHTTATPSATASVLGPSTPRSATSHSISELSPDSEVPRSEGHS

SEQ ID NO: 31 - protein, 565 aa, Homo sapiens
MRPRSALPRLLLPLLLLPAAGPAQFHGEKGISIPDHGFCQPISIPLCTDIAYNQTIMPNLLGHTNQEDAGLEVHQFYPLVKVQCSPELRFFLCSMYAPVCTVLEQAIPPCRSICERARQGCEALMNKFGFQWPERLRCEHFPRHGAEQICVGQNHSEDGAPALLTTAPPPGLQPGAGGTPGGPGGGGAPPRYATLEHPFHCPRVLKVPSYLSYKFLGERDCAAPCEPARPDGSMFFSQEETRFARLWILTWSVLCCASTFFTVTTYLVDMQRFRYPERPIIFLSGCYTMVSVAYIAGFVLQERVVCNERFSEDGYRTVVQGTKKEGCTILFMMLYFFSMASSIWWVILSLTWFLAAGMKWGHEAIEANSQYFHLAAWAVPAVKTITILAMGQIDGDLLSGVCFVGLNSLDPLRGFVLAPLFVYLFIGTSFLLAGFVSLFRIRTIMKHDGTKTEKLERLMVRIGVFSVLYTVPATIVIACYFYEQAFREHWERSWVSQHCKSLAIPCPAHYTPRMSPDFTVYMIKYLMTLIVGITSGFWIWSGKTLHSWRKFYTRLTNSRHGETTV

SEQ ID NO: 32 - protein, 524 aa, Homo sapiens
MDTESTYSGYSYYSSHSKKSHRQGERTRERHKSPRNKDGRGSEKSVTIQPPTGEPLLGNDSTRTEEVQDDNWGETTTAITGTSEHSISQEDIARISKDMEDSVGLDCKRYLGLTVASFLGLLVFLTPIAFILLPPILWRDELEPCGTICEGLFISMAFKLLILLIGTWALFFRKRRADMPRVFVFRALLLVLIFLFVVSYWLFYGVRILDSRDRNYQGIVQYAVSLVDALLFIHYLAIVLLELRQLQPMFTLQVVRSTDGESRFYSLGHLSIQRAALVVLENYYKDFTIYNPNLLTASKFRAAKHMAGLKVYNVDGPSNNATGQSRAMIAAAARRRDSSHNELYYEEAEHERRVKKRKARLVVAVEEAFIHIQRLQAEEQQKAPGEVMDPREAAQAIFPSMARALQKYLRITRQQNYHSMESILQHLAFCITNGMTPKAFLERYLSAGPTLQYDKDRWLSTQWRLVSDEAVTNGLRDGIVFVLKCLDFSLVVNVKKIPFIILSEEFIDPKSHKFVLRLQSETSV

SEQ ID NO: 33 - protein, 521 aa, Homo sapiens
MDTESQYSGYSYKSGHSRSSRKHRDRRDRHRSKSRDGGRGDKSVTIQAPGEPLLDNESTRGDERDDNWGETTTVVTGTSEHSISHDDLTRIAKDMEDSVPLDCSRHLGVAAGATLALLSFLTPLAFLLLPPLLWREELEPCGTACEGLFISVAFKLLILLLGSWALFFRRPKASLPRVFVLRALLMVLVFLLVVSYWLFYGVRILDARERSYQGVVQFAVSLVDALLFVHYLAVVLLELRQLQPQFTLKVVRSTDGASRFYNVGHLSIQRVAVWILEKYYHDFPVYNPALLNLPKSVLAKKVSGFKVYSLGEENSTNNSTGQSRAVIAAAARRRDNSHNEYYYEEAEHERRVRKRRARLVVAVEEAFTHIKRLQEEEQKNPREVMDPREAAQAIFASMARAMQKYLRTTKQQPYHTMESILQHLEFCITHDMTPKAFLERYLAAGPTIQYHKERWLAKQWTLVSEEPVTNGLKDGIVFLLKRQDFSLVVSTKKVPFFKLSEEFVDPKSHKFVMRLQSETSV

SEQ ID NO: 34 - protein, 610 aa, Homo sapiens
MRGAARLGRPGRSCLPGARGLRAPPPPPLLLLLALLPLLPAPGAAAAPAPRPPELQSASAGPSVSLYLSEDEVRRLIGLDAELYYVRNDLISHYALSFSLLVPSETNFLHFTWHAKSKVEYKLGFQVDNVLAMDMPQVNISVQGEVPRTLSVFRVELSCTGKVDSEVMILMQLNLTVNSSKNFTVLNFKRRKMCYKKLEEVKTSALDKNTSRTIYDPVHAAPTTSTRVFYISVGVCCAVIFLVAIILAVLHLHSMKRIELDDSISASSSSQGLSQPSTQTTQYLRADTPNNATPITSSLGYPTLRIEKNDLRSVTLLEAKGKVKDIAISRERITLKDVLQEGTFGRIFHGILIDEKDPNKEKQAFVKTVKDQASEIQVTMMLTESCKLRGLHHRNLLPITHVCIEEGEKPMVILPYMNWGNLKLFLRQCKLVEANNPQAISQQDLVHMAIQIACGMSYLARREVIHKDLAARNCVIDDTLQVKITDNALSRDLFPMDYHCLGDNENRPVRWMALESLVNNEFSSASDVWAFGVTLWELMTLGQTPYVDIDPFEMAAYLKDGYRIAQPINCPDELFAVMACCWALDPEERPKFQQLVQCLTEFHAALGAYV

SEQ ID NO: 35 - protein, 205 aa, Homo sapiens
VAARSFPPSSFVMFRGLRQRFHLTLSLSFATVQQSGLLFYNGRLNEKHDFLALELVAGQVRLTYSTGESNTVVSPTVPGGLSDGQWHTVHLRYYNKPRTDALGGAQGPSKDKVAVLSVDDCDVAVALQFGAEIGNYSCAAAGVQTSSKKSLDLTGPLLLGGVPNLPENFPVSHKDFIGCMRDLHIDGRRVDMAAFVANNGTMAGC

SEQ ID NO: 36 - protein, 204 aa, Mus musculus
VAARSFPPSSFVMFRGLRQRFHLTLSLSFATVQPSGLLFYNGRLNEKHDFLALELVAGQVRLTYSTGESNTVVSPTVPGGLSDGQWHTVHLRYYNKPRTDALGGAQGPSKDKVAVLSVDDCNVAVALQFGAEIGNYSCMAGVQTSSKKSLDLTGPLLLGGVPNLPENFPVSHKDFIGCMRDLHIDGRRMDMAAFVANNGTMAGC

SEQ ID NO: 37 - protein, 33 aa, Homo sapiens
HRMDQQCPRGWWGSPTCGPCNCDVHKGFDPNCN

SEQ ID NO: 38 - protein, 40 aa, Mus musculus
YFGQHCEHRVDQQCPRGWWGSPTCGPCNCDVHKGFDPNCN

SEQ ID NO: 39 - protein, 36 aa, Homo sapiens
TNGQCHCKEFHYRPRGSDSCLPCDCYPVGSTSRSCA

SEQ ID NO: 40 - protein, 330 aa, Homo sapiens [sequence omitted]

SEQ ID NO: 41 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK